(19) United States
(12) Patent Application Publication
(10) Pub. No.: US 2003/0051266 A1
(43) Pub. Date: Mar. 13, 2003

(54) Collections of transgenic animal lines (living library)

(76) Inventor: Tito Andrew Serafini, San Mateo, CA (US)

Correspondence Address: PENNIE AND EDMONDS, 1155 AVENUE OF THE AMERICAS, NEW YORK, NY, 100362711

(21) Appl. No.: 09/783,487

(22) Filed: Feb. 14, 2001

(51) Int. Cl. A01K 67/027; G01N 33/53

(52) U.S. Cl. 800/18; 435/7.1

(57) ABSTRACT

The invention provides collections of transgenic animals and vectors for producing transgenic animals, which transgenic animals and vectors have a transgene comprising sequences encoding a detectable or selectable marker, the expression of which marker is under the control of regulatory sequences from an endogenous gene such that when the transgene is present in the genome of the transgenic animal, the detectable or selectable marker has the same expression pattern as the endogenous gene. Such transgenic animals can then be used to detect, isolate and/or select pure populations of cells having a particular functional characteristic. The isolated cells have uses in gene discovery, target identification and validation, genomic and proteomic analysis, etc.

### 1. TECHNICAL FIELD

The present invention relates to methods for producing transgenic animal lines and vectors for producing such transgenic animal lines in which a particular subset of cells, characterized by the expression of a particular endogenous gene, expresses a detectable or selectable marker or a protein product that specifically induces or suppresses a detectable or selectable marker. The invention provides collections of such lines of transgenic animals and vectors for producing them, and also provides methods for the detection, isolation and/or selection of a subset of cells expressing the marker gene in such transgenic animal lines.

### 2. BACKGROUND OF THE INVENTION

An important goal in the design and development of new therapies for human diseases and disorders is characterizing the responses of afflicted cell types to candidate therapeutic molecules. The complexity of tissues such as the nervous system, however, poses a challenge for those seeking to identify new therapeutic molecules based on the responses of a particular identified cell type. The enormous heterogeneity of the nervous system (thousands of neuronal cell types) and of cell-specific patterns of gene expression (more genes are expressed in the brain than in any other organ or tissue), as well as the scarcity of relevant cell-based assays for high-throughput screening, are serious barriers to the design and development of new therapies. Few cell types can be isolated in a pure population by dissection and immortalized cell lines derived from a particular cell type are often unavailable or have changed physiologically from the cell type present in an organism.

A technology that would permit more rapid recognition, identification, characterization and/or isolation of pure populations of a particular cell type would, therefore, have broad application to numerous types of experimental protocols, both in vivo and in vitro, for example, pharmacological, behavioral, physiological, and electrophysiological assays, drug discovery assays, target validation assays, etc.

A particular cell type can be classified, inter alia, by the specific subset of genes it expresses out of the total number of genes in the genome. Identification of a cell type based on the analysis of its patterns of gene expression among the cells of an organism can be laborious, however, in the absence of easily recognized genetic or molecular markers, such as markers that are detectable by human eye or by an automated detector or cell sorting apparatus.

Once a particular cell type is identified among the cells of an organism, the genes that impart functionally relevant properties to that cell type and the responses of the cells to experimental treatments can be recognized and assayed more easily. The ability to identify and isolate distinct cell types within an organism systematically based upon the expression of a marker gene driven by an endogenous gene would enable, e.g., drug-discovery assays in which the expression pattern of a gene in a known cell type that potentially encodes a drug target may be monitored. We describe such a technology here.

### 3. SUMMARY OF THE INVENTION

The invention provides lines of transgenic animals, preferably mice, in which a subset of cells characterized by expression of a particular endogenous gene (a “characterizing gene”) expresses, either constitutively or conditionally, a “system gene,” which preferably encodes a detectable or selectable marker or a protein product that induces or suppresses the expression of a detectable or selectable marker, allowing detection, isolation and/or selection of the subset of cells from the other cells of the transgenic animal, or explanted tissue thereof. In a preferred embodiment, the transgene introduced into the transgenic animal includes at least the coding region sequences for the system gene product operably linked to all or a portion of the regulatory sequences from the characterizing gene such that the system gene has the same pattern of expression within the animal (i.e., is expressed substantially in the same population of cells) or within the anatomical region containing the cells to be analyzed as the characterizing gene. Also, preferably, the transgene containing the system gene coding sequences and characterizing gene sequences is present in the genome at a site other than where the endogenous characterizing gene is located. In preferred embodiments, the invention provides such lines of transgenic animals in which the characterizing gene is one of the genes listed in Tables 1-15, infra.

The invention further provides methods of producing such transgenic animals and vectors for producing such transgenic animals. In particular, each transgenic line is created by the introduction, for example by pronuclear injection, of a vector containing the transgene into a founder animal, such that the transgene is transmitted to offspring in the line. The transgene preferably randomly integrates into the genome of the founder but in specific embodiments may be introduced by directed homologous recombination. In a preferred embodiment, homologous recombination in bacteria is used for target-directed insertion of the system gene sequence into the genomic DNA for all or a portion of the characterizing gene, including sufficient characterizing gene regulatory sequences to promote expression of the characterizing gene in its endogenous expression pattern. In a preferred embodiment, the characterizing gene sequences are on a bacterial artificial chromosome (BAC). In specific embodiments, the system gene coding sequences are inserted as a 5′ fusion with the characterizing gene coding sequence such that the system gene coding sequences are inserted in frame and directly 3′ from the initiation codon for the characterizing gene coding sequences. In another embodiment, the system gene coding sequences are inserted into the 3′ untranslated region (UTR) of the characterizing gene and, preferably, have their own internal ribosome entry sequence (IRES).

The vector (preferably a BAC) comprising the system gene coding sequences and characterizing gene sequences is then introduced into the genome of a potential founder animal to generate a line of transgenic animals. Potential founder animals can be screened for the selective expression of the system gene sequence in the population of cells characterized by expression of the endogenous characterizing gene. Transgenic animals that exhibit appropriate expression (e.g., detectable expression of the system gene product having the same expression pattern within the animal as the endogenous characterizing gene) are selected as founders for a line of transgenic animals.

In preferred embodiments, the invention provides a collection of such transgenic animal lines comprising at least two individual lines, preferably at least five individual lines more preferably at least fifty individual lines, where the characterizing gene is different for each of said transgenic animal lines. In other preferred embodiments, the invention provides a collection of at least two, five, ten, fifty or one hundred vectors (preferably BACs) for producing such transgenic animal lines wherein the characterizing gene is different for each said vector in the collection. Each individual line or vector is selected for the collection based on the identity of the subset of cells in which the system gene is expressed. In a preferred embodiment, the characterizing genes for the lines of transgenic animals or vectors in such a collection consist of (or comprise), for example but not by way of limitation, a group of functionally related genes (i.e., genes encoding proteins that serve analogous functions in the cells in which they are expressed, such as proteins that function in the cell as biosynthetic and/or degradative enzymes for a cellular component, transporters, intracellular or extracellular receptors, and signal transduction molecules, etc.), a group of genes in the same signal transduction pathway, or a group of genes implicated in a particular physiological or disease state. Additionally, the collection may consist of lines of transgenic animals in which the characterizing genes represent a battery of genes having a variety of cell functions, are expressed in a variety of tissue or cell types (e.g., different neuronal cell types, different brain cell types, etc.), or are implicated in a variety of hysiological or disease states. In a preferred embodiment, a group of functionally related genes that are characterizing genes encode the cellular components associated with a biosynthesis and/or function of a neurotransmitter, a cell signaling pathway, a disease state, a known neuronal circuitry, or a physiological or behavioral state or response. Such states or responses include pain, sleeping, feeding, fasting, sexual behavior, aggression, depression, cognition, emotion, etc.

In a specific embodiment, the invention provides one or more lines of transgenic animals where the transgenic animals contain two or more transgenes of the invention, each transgene having a different characterizing gene and the transgenes having the same or different system genes.

The collections of transgenic animal lines and/or vectors of the invention may be used for the identification and isolation of pure populations of particular classes of cells. The invention further provides such isolated cells. Such cells can be, for example, derived from a particular tissue or associated with a particular physiological, behavioral or disease state. In a preferred embodiment, the isolated cells are associated with a particular neurotransmitter pathway, cell signaling pathway, disease state, known neuronal circuitry, or physiological or behavioral state or response. Such states or responses include pain, sleeping, feeding, fasting, sexual behavior, aggression, depression, cognition, emotion, etc.

The invention further provides methods of using such isolated cells in assays such as drug screening assays, pharmacological, behavioral, and physiological assays, and genomic analysis.

### 4. DETAILED DESCRIPTION OF THE INVENTION

For clarity of disclosure, and not by way of limitation, the detailed description of the invention is divided into the subsections set forth below.

4.1. Transgenic Animal Lines and Collections of Transgenic Animal Lines

The invention provides transgenic animal lines and vectors for producing transgenic animal lines of the invention. Each transgenic line of the collections of the invention is created by the introduction of a transgene into a founder animal, such that the transgene is transmitted to offspring in the line. A line may include transgenic animals derived from more than one founder animal but that contain the same transgene, preferably in the same chromosomal position and/or exhibiting the same level and pattern of expression within the organism. For example, in certain circumstances, it may be necessary to use more than one founder to maintain or rederive a line. In each transgenic animal line, a subset of cells of he transgenic animal that is characterized by expression of a particular endogenous gene (a “characterizing gene”) also expresses, either constitutively or conditionally, a “system gene,” which preferably encodes a detectable or selectable marker or a protein product that specifically induces or suppresses the expression of a detectable or selectable marker.

In preferred embodiments, the invention provides a collection of such transgenic animal lines comprising at least two individual lines, and preferably, at least five individual lines. In specific embodiments, a collection of transgenic animal lines comprises at least 10, 15, 20, 25, 30, 35, 40, 45, 50, 75, 100, 200, 500, 1000, or 2000 individual lines. In other embodiments, a collection of transgenic animal lines comprises between 2 to 10, 10 to 20, 10 to 50, 10 to 100, 100 to 500, 100 to 1000, or 100 to 2000 individual lines. In the collections, each line of transgenic animals has a different characterizing gene and may or may not have different system gene coding sequences. In particular embodiments, each transgenic animal line of a collection of the invention has the same system gene coding sequences and in other embodiments, each transgenic animal line has a different system gene coding sequence.

In other preferred embodiments, the invention provides a collection of vectors for producing transgenic animal lines of the invention comprising at least two vectors, and preferably, at least five vectors. In specific embodiments, a collection of vectors comprises at least 10, 15, 20, 25, 30, 35, 40, 45, 50, 75, 100, 200, 500, 1000, or 2000 vectors. In other embodiments, a collection of vectors comprises between 2 to 10, 10 to 20, 10 to 50, 10 to 100, 100 to 500, 100 to 1000, or 100 to 2000 individual vectors. In the collection of vectors of the invention, the characterizing gene for each vector is different and each vector may or may not have different system gene coding sequences. In particular embodiments, each vector has the same system gene coding sequences and in other embodiments, each vector has a different system gene coding sequence.

Each individual line or vector is selected for the collection of transgenic animals lines and/or vectors based on the identity of the subset of cells in which the system gene is expressed. In a preferred embodiment, the characterizing genes for the lines of transgenic animals in such a collection consist of (or comprise), for example but not by way of limitation, a group of functionally related genes (i.e., genes encoding proteins that serve analogous functions in the cells in which they are expressed such as proteins that function in the cell as biosynthetic and/or degradative enzymes for a cellular component, transporters, intracellular or extracellular receptors, and signal transduction molecules), a group of genes in the same signal transduction pathway, or a group of genes implicated in a particular physiological or disease state, or in the same or related tissue types. Additionally, the collection may consist of lines of transgenic animals in which the characterizing genes represent a battery of genes having a variety of cell functions, are expressed in a variety of tissue or cell types (e.g., different neuronal cell types, different immune system cell types, different tumor cell types, etc.), or are implicated in a variety of physiological or disease states (in particular, related disease states such as a group of different neurodegenerative diseases, cancers, autoimmune diseases or disorders of immune system function, heart diseases, etc.). The collection may also consist of lines of transgenic animals in which the characterizing genes represent a battery of genes expressed in particular neuronal cell types and circuits that control particular behaviors and underlie specific neurological or psychiatric diseases.

In preferred embodiments, the characterizing genes are a group of functionally related genes that encode the cellular components associated with a particular neurotransmitter signaling and/or synthetic pathway or with a particular signal transduction pathway, or the proteins that serve analogous functions in the cells in which they are expressed, such as proteins that function in the cell as biosynthetic and/or degradative enzymes for a cellular component, transporters, intracellular or extracellular receptors, signal transduction molecules, transcriptional or translational regulators, cell cycle regulators, etc. Additionally, the group of functionally related genes that are characterizing genes can be implicated in a particular physiological, behavioral or disease state.

The collection may consist of lines of transgenic animals or vectors for production of transgenic animals in which the characterizing genes represent a battery of genes having a variety of cell functions, are expressed in a variety of tissue or cell types (e.g., different neuronal cell types, different immune system cell types, different tumor cell types, etc.), or are implicated in a variety of physiological or disease states. In a preferred embodiment, a group of functionally related genes that are characterizing genes encode the cellular components associated with a neurotransmitter pathway, a cell signaling pathway, a disease state, a known neuronal circuitry, or a physiological or behavioral state or response. Such states or responses include pain, sleeping, feeding, fasting, sexual behavior, aggression, depression, cognition, emotion, etc.

In one embodiment, the collection of transgenic animal lines or vectors for production of transgenic animal lines has as characterizing genes a group of genes that are functionally related. Such functionally related genes can include, e.g., genes that encode proteins that function in the cell as biosynthetic and/or degradative enzymes for a cellular component, transporters, intracellular or extracellular receptors, and signal transduction molecules.

In a preferred embodiment, a group of characterizing genes is a group of functionally related genes that encode a neurotransmitter, its receptors, and associated biosynthetic and/or degradative enzymes for the neurotransmitter.

In other embodiments, the characterizing genes are groups of genes that are expressed in cells of the same or different neurotransmitter phenotypes, in cells known to be anatomically or physiologically connected, cells underlying a particular behavior, cells in a particular anatomical locus (e.g., the dorsal root ganglia, a motor pathway), cells active or quiescent in a particular physiological state, cells affected or spared in a particular disease state, etc.

In other embodiments, the characterizing genes are groups of genes that are expressed in cells underlying a neuropsychiatric disorder such as a disorder of thought and/or mood, including thought disorders such as schizophrenia, schizotypal personality disorder; psychosis; mood disorders, such as schizoaffective disorders (e.g., schizoaffective disorder manic type (SAD-M); bipolar affective (mood) disorders, such as severe bipolar affective (mood) disorder (BP-I), bipolar affective (mood) disorder with hypomania and major depression (BP-II); unipolar affective disorders, such as unipolar major depressive disorder (MDD), dysthymic disorder; obsessive-compulsive disorders; phobias, e.g., agoraphobia; panic disorders; generalized anxiety disorders; somatization disorders and hypochondriasis; and attention deficit disorders.

In other embodiments, the characterizing genes are groups of genes that are expressed in cells underlying a malignancy, cancer or hyperproliferation disorder such as one of the following:

### Malignancies and Related Disorders

Leukemia

acute leukemia

acute lymphocytic leukemia

acute myelocytic leukemia

myeloblastic

promyelocytic

myelomonocytic

monocytic

erythroleukemia

chronic leukemia

chronic myelocytic (granulocytic) leukemia

chronic lymphocytic leukemia

Polycythemia vera

Lymphoma

Hodgkin's disease

non-Hodgkin's disease

Multiple myeloma

Waldenström's macroglobulinemia

Heavy chain disease

Solid tumors

sarcomas and carcinomas

fibrosarcoma

myxosarcoma

liposarcoma

chondrosarcoma

osteogenic sarcoma

chordoma

angiosarcoma

endotheliosarcoma

lymphangiosarcoma

lymphangioendotheliosarcoma

synovioma

mesothelioma

Ewing's tumor

leiomyosarcoma

rhabdomyosarcoma

colon carcinoma

pancreatic cancer

breast cancer

ovarian cancer

prostate cancer

squamous cell carcinoma

basal cell carcinoma

adenocarcinoma

sweat gland carcinoma

sebaceous gland carcinoma

papillary carcinoma

papillary adenocarcinomas

cystadenocarcinoma

medullary carcinoma

bronchogenic carcinoma

renal cell carcinoma

hepatoma

bile duct carcinoma

choriocarcinoma

seminoma

embryonal carcinoma

Wilms' tumor

cervical cancer

uterine cancer

testicular tumor

lung carcinoma

small cell lung carcinoma

bladder carcinoma

epithelial carcinoma

glioma

astrocytoma

medulloblastoma

craniopharyngioma

ependymoma

pinealoma

hemangioblastoma

acoustic neuroma

oligodendroglioma

menangioma

melanoma

neuroblastoma

retinoblastoma

In another embodiment, the characterizing genes of the collection are all expressed in the same population of cells, e.g., motorneurons of the spinal cord, amacrine cells, astroglia, etc.

In another embodiment, the characterizing genes of the collection are expressed in different populations of cells.

In another embodiment, the characterizing genes of the collection are all expressed within a particular anatomical region, tissue, or organ of the body, e.g., nucleus within the brain or spinal cord, cerebral cortex, cerebellum, retina, spinal cord, bone marrow, skeletal muscles, smooth muscles, pancreas, thymus, etc.

In another embodiment, the characterizing genes of the collection are each expressed in a different anatomical region, tissue, or organ of the body.

In another embodiment, the characterizing genes of the collection are all listed in one of Tables 1-15 below.

In another embodiment, the characterizing genes of the collection are a group of genes where at least two, three, five, eight, ten or twelve genes are each from a different one of Tables 1-15 below.

In another embodiment, in the collection, at least one characterizing gene is listed in one of Tables 1-15 below.

In another embodiment, the characterizing genes of the collection comprise at least one gene from each of one, two, three, four or more of Tables 1-15 below.

In another embodiment, the characterizing genes of the collection are all expressed temporally in a particular expression pattern during an organism's development.

In another embodiment, the characterizing genes of the collection are all expressed during the display of a temporally rhythmic behavior, such as a circadian behavior, a monthly behavior, an annual behavior, a seasonal behavior, or estrous or other mating behavior, or other periodic or episodic behavior.

In another embodiment, the characterizing genes of the collection are all expressed in cells of the nervous system that underlie feeding behavior. In a specific embodiment, the characterizing genes of the collection are all expressed in neuronal circuits that function as positive and negative regulators of feeding behavior and, preferably, that are located in the hypothalamus.

In specific preferred embodiments, the invention provides vectors and lines of transgenic animals in which the characterizing gene is one of the genes listed in any of Tables 1-15, infra.

In other embodiments, the invention provides lines of transgenic animals, wherein each transgenic animal contains two, four, five, six, seven, eight, ten, twelve, fifteen, twenty or more transgenes of the invention (i.e., containing system gene coding sequences operably linked to characterizing gene regulatory sequences). Each of the transgenes has a different characterizing gene. In a specific embodiment, all of the transgenes in the line of transgenic animals contain the same system gene coding sequences. In another embodiment, the transgenes in the line of transgenic animals have different system gene coding sequences (i.e., the cells expressing the different characterizing genes express a different detectable or selectable marker). Such lines of transgenic animals may be generated by introducing a transgene into an animal that is already transgenic for a transgene of the invention or by breeding two animals transgenic for a transgene of the invention. Once a line of transgenic animals containing two transgenes of the invention is established, additional transgenes can be introduced into that line, for example, by pronuclear injection or by breeding, to generate a line of transgenic animals transgenic for three transgenes of the invention, and so on.

The transgenic animal lines and collections of transgenic animal lines of the invention and collections of vectors of the invention may be used for the identification and isolation of pure populations of particular classes of cells, which then may be used for pharmacological, behavioral, physiological, electrophysiological, drug discovery assays, target validation, gene expression analysis, etc.

In certain embodiments, the response of a particular cell type to the presence of a test substance or physiological state can be assessed. Such response could be, for example, the response of a dopaminergic (DA) neuron to the presence of a candidate antipsychotic drug, the response of a serotonergic neuron to a candidate antidepressive drug, the response of an agouti-related protein (AGRP)-positive neuron to fasting, etc.

4.2. Transgenes

Each transgenic animal line of the invention contains a transgene which comprises system gene coding sequences under the control of the regulatory sequences for a characterizing gene such that the system gene has substantially the same expression pattern as the endogenous characterizing gene. The expression of the system gene marker permits detection, isolation and/or selection of the population of cells expressing the system gene from the other cells of the transgenic animal, or explanted tissue thereof or dissociated cells thereof.

A transgene is a nucleotide sequence that has been or is designed to be incorporated into a cell, particularly a mammalian cell, that in turn becomes or is incorporated into a living animal such that the nucleic acid containing the nucleotide sequence is expressed (i.e., the mammalian cell is transformed with the transgene). The characterizing gene sequence is preferably endogenous to the transgenic animal, or is an ortholog of an endogenous gene, e.g., the human ortholog of a gene endogenous to the animal to be made transgenic. A transgene may be present as an extrachromosomal element in some or all of the cells of a transgenic animal or, preferably, stably integrated into some or all of the cells, more preferably into the germline DNA of the animal (i. e., such that the transgene is transmitted to all or some of the animal's progeny), thereby directing expression of an encoded gene product (i.e., the system gene product) in one or more cell types or tissues of the transgenic animal. Unless otherwise indicated, it will be assumed that a transgenic animal comprises stable changes to the chromosomes of germline cells. In a preferred embodiment, the transgene is present in the genome at a site other than where the endogenous characterizing gene is located. In other embodiments, the transgene is incorporated into the genome of the transgenic animal at the site of the endogenous characterizing gene, for example, by homologous recombination.

Such transgenic animals are created by introducing a transgenic construct of the invention into its genome using methods routine in the art, for example, the methods described in Section 4.4 and 4.5, infra, and using the vectors described in Section 4.3, infra. A construct is a recombinant nucleic acid, generally recombinant DNA, generated for the purpose of the expression of a specific nucleotide sequence(s), or is to be used in the construction of other recombinant nucleotide sequences. A transgenic construct of the invention includes at least the coding region for a system gene operably linked to all or a portion of the regulatory sequences, e.g a promoter and/or enhancer, of the characterizing gene. The transgenic construct optionally includes enhancer sequences and coding and other non-coding sequences (including intron and 5′ and 3′ untranslated sequences) from the characterizing gene such that the system gene is expressed in the same subset of cells as the characterizing gene. The system gene coding sequences and the characterizing gene regulatory sequences are operably linked, meaning that they are connected in such a way so as to permit expression of the system gene when the appropriate molecules (e.g., transcriptional activator proteins) are bound to the characterizing gene regulatory sequences. Preferably the linkage is covalent, most preferably by a nucleotide bond. The promoter region is of sufficient length to promote transcription, as described in Alberts et al. (1989) in Molecular Biology of the Cell, 2d Ed. (Garland Publishing, Inc.). In one aspect of the invention, the regulatory sequence is the promoter of a characterizing gene. Other promoters that direct tissue-specific expression of the coding sequences to which they are operably linked are also contemplated in the invention. In specific embodiments, a promoter from one gene and other regulatory sequences (such as enhancers) from other genes are combined to achieve a particular temporal and spatial expression pattern of the system gene.

In a specific embodiment, the system gene coding sequences code for a protein that activates, enhances or suppresses the expression of a detectable or selectable marker. More particularly, the transgene comprises the system gene coding sequences operably linked to characterizing gene regulatory sequences and further comprises sequences encoding a detectable or selectable marker operably linked to an expression control element that is activatable or suppressible by the protein product of the system gene coding sequences. In other embodiments, the sequences encoding the detectable or selectable marker operably linked to sequences that activate or suppress expression of the marker in the presence of the system gene protein product are present on a second transgene introduced into the transgenic animal containing the transgene with the system gene operably linked to the characterizing gene regulatory sequences, for example, but not by way of limitation, by random integration directly into the genome of the transgenic animal or by breeding with a transgenic animal of the invention.

Methods that are well known to those skilled in the art can be used to construct vectors containing system gene coding sequences operatively associated with the appropriate transcriptional and translational control signals of the characterizing gene (see Section 4.2.1, infra). These methods include, for example, in vitro recombinant DNA techniques and in vivo genetic recombination. See, for example, the techniques described in Sambrook et al., 2001, Molecular Cloning, A Laboratory Manual, Third Edition, Cold Spring Harbor Laboratory Press, N.Y.; and Ausubel et al., 1989, Current Protocols in Molecular Biology, Green Publishing Associates and Wiley Interscience, N.Y., both of which are hereby incorporated by reference in their entireties.

The system gene coding sequences may be incorporated into some or all of the characterizing gene sequences such that the system gene is expressed in substantially the same expression pattern as the endogenous characterizing gene in the transgenic animal or at least in the anatomical region or tissue of the animal (by way of example, in the brain, spinal chord, heart, skin, bones, head, limbs, blood, muscle, peripheral nervous system, etc.) containing the population of cells to be marked by expression of the system gene coding sequences so that tissue can be dissected from the transgenic mouse which contains only cells of interest expressing the system gene coding sequences. By “substantially the same expression pattern” is meant that the system gene coding sequences are expressed in at least 35 80%, 85%, 90%, 95%, and preferably 100% of the cells shown to express the endogenous characterizing gene by in situ hybridization. Because detection of the system gene expression product may be more sensitive than in situ hybridization detection of the endogenous characterizing gene messenger RNA, more cells may be detected to express the system gene product in the transgenic mice of the invention than are detected to express the endogenous characterizing gene by in situ hybridization or any other method known in the art for in situ detection of gene expression.

For example, the nucleotide sequences encoding the system gene protein product may replace the characterizing gene coding sequences in a genomic clone of the characterizing gene, leaving the characterizing gene regulatory non-coding sequences. In other embodiments, the system gene coding sequences (either genomic or cDNA sequences) replace all or a portion of the characterizing gene coding sequence and the transgene only contains the upstream and downstream characterizing gene regulatory sequences.

In a preferred embodiment, the system gene coding sequences are inserted into or replace transcribed coding or non-coding sequences of the genomic characterizing gene sequences, for example, into or replacing a region of an exon or of the 3′ UTR of the characterizing gene genomic sequence. Preferably, the system gene coding sequences are not inserted into or replace regulatory sequences of the genomic characterizing gene sequences. Preferably, the system gene coding sequences are also not inserted into or replace characterizing gene intron sequences.

In a preferred embodiment, the system gene coding sequence is inserted into or replaces a portion of the 3′ untranslated region (UTR) of the characterizing gene genomic sequence. In another preferred embodiment, the coding sequence of the characterizing gene is mutated or disrupted to abolish characterizing gene expression from the transgene without affecting the expression of the system gene. Preferably, the system gene coding sequence has its own internal ribosome entry site (IRES). For descriptions of IRESes, see, e.g., Jackson et al., 1990, Trends Biochem Sci. 15(12):477-83; Jang et al., 1988, J. Virol. 62(8):2636-43; Jang et al., 1990, Enzyme 44(1-4):292-309; and Martinez-Salas, 1999, Curr. Opin. Biotechnol. 10(5):458-64.

In another embodiment, the system gene is inserted at the 3′ end of the characterizing gene coding sequence. In a specific embodiment, the system coding sequences are introduced at the 3′ end of the characterizing gene coding sequence such that the transgene encodes a fusion of the characterizing gene and the system gene sequences. In a specific embodiment, the system gene coding sequences encode an epitope tag.

Preferably, the system gene coding sequences are inserted using 5′ direct fusion wherein the system gene coding sequences are inserted in-frame adjacent to the initial ATG sequence (or adjacent the nucleotide sequence encoding the first two, three, four, five, six, seven or eight amino acids of the characterizing gene protein product) of the characterizing gene, so that translation of the inserted sequence produces a fusion protein of the first methionine (or first few amino acids) derived from the characterizing gene sequence fused to the system gene protein. In this embodiment, the characterizing gene coding sequence 3′ of the system gene coding sequences are not expressed. In yet another specific embodiment, a system gene is inserted into a separate cistron in the 5′ region of the characterizing gene genomic sequence and has an independent IRES sequence.

In certain embodiments, an IRES is operably linked to the system gene coding sequence to direct translation of the system gene. The IRES permits the creation of polycistronic mRNAs from which several proteins can be synthesized under the control of an endogenous transcriptional regulatory sequence. Such a construct is advantageous because it allows marker proteins to be produced in the same cells that express the endogenous gene (Heintz, 2000, Hum. Mol. Genet. 9(6): 937-43; Heintz et al., WO 98/59060; Heintz et al., WO 01/05962; which are incorporated herein by reference in their entireties).

Shuttle vectors containing an IRES, such as the pLD55 shuttle vector (see Heintz et al., WO 01/05962), may be used to insert the system gene sequence into the characterizing gene. The IRES in the pLD55 shuttle vector is derived from EMCV (encephalomyocarditis virus) (Jackson et al., 1990, Trends Biochem Sci. 15(12):477-83; and Jang et al., 1988, J. Virol. 62(8):2636-43, both of which are hereby incorporated by reference). The common sequence between the first and second IRES sites in the shuttle vector is shown below. This common sequence also matches pIRES (Clontech) from 1158-1710.

| | |
|---|---|
| TAACGTTACTGGCCGAAGCCGCTTGGAATAAGGCCGGTGTGCGTTTGTCTATAT | (SEQ ID NO:1) |
| GTTATTTTCCACCATATTGCCGTCTTTTGGCAATGTGAGGGCCCGGAAACCTGG | |
| CCCTGTCTTCTTGACGAGCATTCCTAGGGGTCTTTCCCCTCTCGCCAAAGGAATG | |
| CAAGGTCTGTTGAATGTCGTGAAGGAAGCAGTTCCTCTGGAAGCTTCTTGAAGA | |
| CAAACAACGTCTGTAGCGACCCTTTGCAGGCAGCGGAACCCCCCACCTGGCGA | |
| CAGGTGCCTCTGCGGCCAAAAGCCACGTGTATAAGATACACCTGCAAAGGCGG | |
| CACAACCCCAGTGCCACGTTGTGAGTTGGATAGTTGTGGAAAGAGTCAAATGG | |
| CTCTCCTAAGCGTATTCAACAAGGGGCTGAAGGATGCCCAGAAGGTACTCCATT | |
| GTATGGGATCTGATCTGGGGCCTCGGTGCACATGCTTTACATGTGTTTAGTCGA | |
| GGTTAAAAAAACGTCTAGGCCCCCCGAACCACGGGGACGTGGTTTTCCTTTGAA | |
| AAACACCATGATA | |

In a specific embodiment, the EMCV IRES is used to direct independent translation of the system gene coding sequences (Gorski and Jones, 1999, Nucleic Acids Research 27(9):2059-61).

In another embodiment, more than one IRES site is present in the transgene to direct translation of more than one coding sequence. However, in this case, each IRES sequence must be a different sequence.

In certain embodiments where a system gene is expressed conditionally, the system gene coding sequence is embedded in the genomic sequence of the characterizing gene and is inactive unless acted on by a transactivator or recombinase, whereby expression of the system gene can then be driven by the characterizing gene regulatory sequences.

In other embodiments, a marker gene is expressed conditionally, through the activity of the system gene which is an activator or suppressor of gene expression. In this case, the system gene encodes a transactivator, e.g., tetR, or a recombinase, e.g., FLP, whose expression is regulated by the characterizing gene regulatory sequences. The marker gene is linked to a conditional element, e.g., the tet promoter, or is flanked by recombinase sites, e.g., FRT sites, and may be located anywhere within the genome. In such a system, expression of the system gene, as regulated by the characterizing gene regulatory sequences, activates the expression of the marker gene.

In certain embodiments, exogenous translational control signals, including, for example, the ATG initiation codon, can be provided by the characterizing gene or some other heterologous gene. The initiation codon must be in phase with the reading frame of the desired coding sequence of the system gene to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153: 516-44).

As detailed below in Section 4.3, the construct can also comprise one or more selectable markers that enable identification and/or selection of recombinant vectors. The selectable marker may be the system gene product itself or an additional selectable marker, not necessarily tied to the expression of the characterizing gene.

In a specific embodiment, the transgene is expressed conditionally, using any type of inducible or repressible system available for conditional expression of genes known in the art, e.g., a system inducible or repressible by tetracycline (“tet system”); interferon;

estrogen, ecdysone, or other steroid inducible system; Lac operator, progesterone antagonist RU486, or rapamycin (FK506) (see Section 4.2.3, infra). For example, a conditionally expressible transgene can be created in which the coding region for the system gene (and, optionally also the characterizing gene) is operably linked to a genetic switch, such that expression of the system gene can be further regulated. One example of this type of switch is a tetracycline-based switch (see Section 4.2.3). In a specific embodiment, the system gene product is the conditional enhancer or suppressor which, upon expression, enhances or suppresses expression of a selectable or detectable marker present either in the transgene or elsewhere in the genome of the transgenic animal.

A conditionally expressible transgene can be site-specifically inserted into an untranslated region (UTR) of genomic DNA of the characterizing gene, e.g., the 3′ UTR or the 5′ region, so that expression of the transgene via the conditional expression system is induced or abolished by administration of the inducing or repressing substance, e.g., administration of tetracycline or doxycycline, ecdysone, estrogen, etc., without interfering with the normal profile of gene expression (see, e.g., Bond et al., 2000, Science 289: 1942-46; incorporated herein by reference in its entirety). In the case of a binary system, the detectable or selectable marker operably linked to the conditional expression elements is present in the transgene, but outside the characterizing gene coding sequences and not operably linked to characterizing gene regulatory sequences or, alternatively, on another site in the genome of the transgenic animal.

Preferably, the transgene comprises all or a significant portion of the genomic characterizing gene, preferably, at least all or a significant portion of the 5′ regulatory sequences of the characterizing gene, most preferably, sufficient sequence 5′ of the characterizing gene coding sequence to direct expression of the system gene coding sequences in the same expression pattern (temporal and/or spatial) as the endogenous counterpart of the characterizing gene. In certain embodiments, the transgene comprises one exon, two exons, all but one exon, or all but two exons, of the characterizing gene.

Nucleic acids comprising the characterizing gene sequences and system gene coding sequences can be obtained from any available source. In most cases, all or a portion of the characterizing gene sequences and/or the system gene coding sequences are known, for example, in publicly available databases such as GenBank, UniGene and the Mouse Gnome Informatic (MGI) Database to name just a few (see Section 4.2.1, infra, for further details), or in private subscription databases. With a portion of the sequence in hand, hybridization probes (for filter hybridization or PCR amplification) can be designed using highly routine methods in the art to identify clones containing the appropriate sequences (preferred methods for identifying appropriate BACs are discussed in Sections 4.3 and 5, infra) for example in a library or other source of nucleic acid. If the sequence of the gene of interest from one species is known and the counterpart gene from another species is desired, it is routine in the art to design probes based upon the known sequence. The probes hybridize to nucleic acids from the species from which the sequence is desired, for example, hybridization to nucleic acids from genomic or DNA libraries from the species of interest.

By way of example and not limitation, genomic clones can be identified by probing a genomic DNA library under appropriate hybridization conditions, e.g., high stringency conditions, low stringency conditions or moderate stringency conditions, depending on the relatedness of the probe to the genomic DNA being probed. For example, if the probe and the genomic DNA are from the same species, then high stringency hybridization conditions may be used; however, if the probe and the genomic DNA are from different species, then low stringency hybridization conditions may be used. High, low and moderate stringency conditions are all well known in the art.

Procedures for low stringency hybridization are as follows (see also Shilo and Weinberg, 1981, Proc. Natl. Acad. Sci. USA 78:6789-6792): Filters containing DNA are pretreated for 6 hours at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 hours at 40° C., and then washed for 1.5 hours at 55° C. in a solution containing 2×SSC, 25 mM Tris-HCl (pH 7.4), 5 mM EDTA, and 0.1% SDS. The wash solution is replaced with fresh solution and incubated an additional 1.5 hours at 60° C. Filters are blotted dry and exposed for autoradiography. If necessary, filters are washed for a third time at 65-68° C. and reexposed to film.

Procedures for high stringency hybridizations are as follows: Prehybridization of filters containing DNA is carried out for 8 hours to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 hours at 65° C. in prehybridization mixture containing 100 μg/ml denatured salmon sperm DNA and 5-20×106 cpm of ³²P-labeled probe. Washing of filters is done at 37° C. for 1 hour in a solution containing 2×SSC, 0.01% PVP, 0.01% Ficoll, and 0.01% BSA. This is followed by a wash in 0.1 ×SSC at 50° C. for 45 minutes before autoradiography.

Moderate stringency conditions for hybridization are as follows: Filters containing DNA are pretreated for 6 hours at 55° C. in a solution containing 6×SSC, 5×Denhardt's solution, 0.5% SDS, and 100 μg/ml denatured salmon sperm DNA. Hybridizations are carried out in the same solution and 5-20×10⁶ CPM ³²P-labeled probe is used. Filters are incubated in the hybridization mixture for 18-20 hours at 55° C., and then washed twice for minutes at 60° C. in a solution containing 1×SSC and 0.1% SDS.

With respect to the characterizing gene, all or a portion of the genomic sequence is preferred, particularly, the sequences 5′ of the coding sequence that contain the regulatory sequences. A preferred method for identifying BACs containing appropriate and sufficient characterizing gene sequences to direct the expression of the system gene coding sequences in substantially the same expression pattern as the endogenous characterizing gene is described in Section 5, infra.

Briefly, the characterizing gene genomic sequences are preferably in a vector that can accommodate significant lengths of sequence (for example, 10 kb's of sequence), such as cosmids, YACs, and, preferably, BACs, and encompass at least 50, 70, 80, 100, 120, 150, 200, 250 or 300 kb of sequence that comprises all or a portion of the characterizing gene sequence. The larger the vector insert, the more likely it is to identify a vector that contains the characterizing gene sequences of interest. Vectors identified as containing characterizing gene sequences can then be screened for those that are most likely to contain sufficient regulatory sequences from the characterizing gene to direct expression of the system gene coding sequences in substantially the same pattern as the endogenous characterizing gene. In general, it is preferred to have a vector containing the entire genomic sequence for the characterizing gene. However, in certain cases, the entire genomic sequence cannot be accommodated by a single vector or such a clone is not available. In these instances (or when it is not known whether the clone contains the entire genomic sequence), preferably the vector contains the characterizing gene sequence with the start, i.e., the most 5′ end, of the coding sequence in the approximate middle of the vector insert containing the genomic sequences and/or has at least 20 kb, 30 kb, 40 kb, 50 kb, 60 kb, 80 kb or 100 kb of genomic sequence on either side of the start of the characterizing gene coding sequence. This can be determined by any method known in the art, for example, but not by way of limitation, by sequencing, restriction mapping, PCR amplification assays, etc. In certain cases, the clones used may be from a library that has been characterized (e.g., by sequencing and/or restriction mapping) and the clones identified can be analyzed, for example, by restriction enzyme digestion and compared to database information available for the library. In this way, the clone of interest can be identified and used to query publicly available databases for existing contigs correlated with the characterizing gene coding sequence start site. Such information can then be used to map the characterizing gene coding sequence start site within the clone. Alternatively, the system gene sequences (or any other heterologous sequences) can be targeted to the 5′ end of the characterizing gene coding sequence by directed homologous recombination (for example as described in Sections 4.3 and 5) in such a way that a restriction site unique or at least rare in the characterizing gene clone sequence is introduced. The position of the integrated system gene coding sequences (and, thus, the 5′ end of the characterizing gene coding sequence) can be mapped by restriction endonuclease digestion and mapping. The clone may also be mapped using internally generated fingerprint data and/or by an alternative mapping protocol based upon the presence of restriction sites and the T7 and SP6 promoters in the BAC vector, as described in Section 5, infra.

In certain embodiments, the system gene coding sequences are to be inserted in a site in the characterizing gene sequences other than the 5′ start site of the characterizing gene coding sequences, for example, in the 3′ most translated or untranslated regions. In these embodiments, the clones containing the characterizing gene should be mapped to insure the clone contains the site for insertion in as well as sufficient sequence 5′ of the characterizing gene coding sequences library to contain the regulatory sequences necessary to direct expression of the system gene sequences in the same expression pattern as the endogneous characterizing gene.

Once such an appropriate vector containing the characterizing gene sequences, the system gene can be incorporated into the characterizing gene sequence by any method known in the art for manipulating DNA. In a preferred embodiment, homologous recombination in bacteria is used for target-directed insertion of the system gene sequence into the genomic DNA encoding the characterizing gene and sufficient regulatory sequences to promote expression of the characterizing gene in its endogenous expression pattern, which characterizing gene sequences have been inserted into a BAC (see Section 4.4, infra). The BAC comprising the system gene and characterizing gene sequences is then introduced into the genome of a potential founder animal for generating a line of transgenic animals, using methods well known in the art, e.g., those methods described in Section 4.5, infra. Such transgenic animals are then screened for expression of the system gene coding sequences that mimics the expression of the endogenous characterizing gene. Several different constructs containing transgenes of the invention may be introduced into several potential founder animals and the resulting transgenic animals then screened for the best, (e.g., highest level) and most accurate (best mimicking expression of the endogenous characterizing gene) expression of the system gene coding sequences.

The transgenic construct can be used to transform a host or recipient cell or animal using well known methods, e.g., those described in Section 4.4, infra. Transformation can be either a permanent or transient genetic change, preferably a permanent genetic change, induced in a cell following incorporation of new DNA (i.e., DNA exogenous to the cell). Where the cell is a mammalian cell, a permanent genetic change is generally achieved by introduction of the DNA into the genome of the cell. In one aspect of the invention, a vector is used for stable integration of the transgenic construct into the genome of the cell. Vectors include plasmids, retroviruses and other animal viruses, BACs, YACs, and the like. Vectors are described in Section 4.3, infra.

4.2.1. Chatacterizing Gene Sequences

A characterizing gene is endogenous to a host cell or host organism (or is an ortholog of an endogenous gene) and is expressed or not expressed in a particular select population of cells of the organism. The population of cells comprises a discemable group of cells sharing a common characteristic. Because of its selective expression, the population of cells may be characterized or recognized based on its positive or negative expression of the characterizing gene. As discussed above, accordingly, all or some of the regulatory sequences of the characterizing gene are incorporated into transgenes of the invention to regulate the expression of system gene coding sequences. Any gene which is not constitutively expressed, (i. e., exhibits some spatial or temporal restriction in its expression pattern) can be a characterizing gene.

Preferably, the characterizing gene is a human or mouse gene associated with an adrenergic or noradrenergic neurotransmitter pathway, e.g., one of the genes listed in Table 1; a cholinergic neurotransmitter pathway, e.g, one of the genes listed in Table 2; a dopaminergic neurotransmitter pathway, e.g., one of the genes listed in Table 3; a GABAergic neurotransmitter pathway, e.g, one of the genes listed in Table 4; a glutaminergic neurotransmitter pathway, e.g., one of the genes listed in Table 5; a glycinergic neurotransmitter pathway, e.g., one of the genes listed in Table 6; a histaminergic neurotransmitter pathway, e.g., one of the genes listed in Table 7; a neuropeptidergic neurotransmitter pathway, e.g, one of the genes listed in Table 8; a serotonergic neurotransmitter pathway, e.g., one of the genes listed in Table 9; a nucleotide receptor, e.g., one of the genes listed in Table 10; an ion channel, e.g., one of the genes listed in Table 11; markers of undifferentiated or not fully differentiated cells, preferably nerve cells, e.g., one of the genes listed in Table 12; the sonic hedgehog signaling pathway, e.g., one of the genes in Table 13; calcium binding, e.g., one of the genes listed in Table 14; or a neurotrophic factor receptor, e.g., one of the genes listed in Table 15.

The ion channel encoded by or associated with the characterizing gene is preferably involved in generating and modulating ion flux across the plasma membrane of neurons, including, but not limited to voltage-sensitive and/or cation-sensitive channels, e.g., a calcium, sodium or potassium channel.

In Tables 1-15 that follow, the common names of genes are listed, as well as their GeneCards identifiers (Rebhan et aL, 1997, GeneCards: encyclopedia for genes, proteins and diseases, Weizmann Institute of Science, Bioinformatics Unit and Genome Center (Rehovot, Israel); http://bioinfo.weizmann.ac.il/cards). GenBank accession numbers, UniGene accession numbers, and Mouse Genome Informatics (MGI) Database accession numbers where available are also listed. GenBank is the NIH genetic sequence database, an annotated collection of all publicly available DNA sequences (Benson et al., 2000, Nucleic Acids Res. 28(1): 15-18; http://www.ncbi.nlm.nih.gov/Genbank/index.html). The GenBank accession number is a unique identifier for a sequence record. An accession number applies to the complete record and is usually a combination of a letter(s) and numbers, such as a single letter followed by five digits (e.g., U12345), or two letters followed by six digits (e.g., AF123456).

Accession numbers do not change, even if information in the record is changed at the author's request. An original accession number might become secondary to a newer accession number, if the authors make a new submission that combines previous sequences, or if for some reason a new submission supercedes an earlier record.

UniGene (http://www.ncbi.nlm.nih.gov/UniGene) is an experimental system for automatically partitioning GenBank sequences into a non-redundant set of gene-oriented clusters for cow, human, mouse, rat, and zebrafish. Within UniGene, expressed sequence tags (ESTs) and full-length mRNA sequences are organized into clusters that each represent a unique known or putative gene. Each UniGene cluster contains related information such as the tissue types in which the gene has been expressed and map location. Sequences are annotated with mapping and expression information and cross-referenced to other resources. Consequently, the collection may be used as a resource for gene discovery.

The Mouse Genome Informatics (MGI) Database is sponsored by the Jackson Laboratory (http://www.informaticsjax.org/mgihome). The MGI Database contains information on mouse genetic markers, mRNA and genomic sequence information, phenotypes, comparative mapping data, experimental mapping data, and graphical displays for genetic, physical, and cytogenetic maps.

TABLE 1

| | | MGI Database |
|---|---|---|
| | GenBank and/or UniGene | Accession |
| Gene | Accession Number | Number |
| ADRB1(adrenergic | human: J03019 | MGI: 87937 |
| beta 1) | | |
| ADRB2 (adrenergic | human: M15169 | MGI: 87938 |
| beta 2) | | |
| ADRB3 (adrenergic | human: NM_000025, X70811, | MGI: 87939 |
| beta 3) | X72861, M29932, X70812, | |
| | S53291, X70812 | |
| ADRA1A (adrenergic | human: D25235, U02569, | |
| alpha1a) | AF013261, L31774, U03866 | |
| | guinea pig: AF108016 | |
| ADRA1B (adrenergic | human: U03865, L31773 | MGI: 104774 |
| alpha 1b) | | |
| ADRA1C (adrenergic | human: U08994 | |
| alpha 1c) | mouse: NM_013461 | |
| ADRA1D (adrenergic | human: M76446, U03864, | MGI: 106673 |
| alpha1d) | L31772, D29952, S70782 | |
| ADRA2A (adrenergic | human: M18415, M23533 | MGI: 87934 |
| alpha2A) | | |
| ADRA2B (adrenergic | human: M34041, AF005900 | MGI: 87935 |
| alpha 2B) | | |
| ADRA2C (adrenergic | human: J03853, D13538, | MGI: 87936 |
| alpha 2C) | U72648 | |
| SLC6A2 | human: X91117, M65105, | MGI: 1270850 |
| Norepinephrine | AB022846, AF061198 | |
| transporter (NET) | | |

TABLE 2

| | | MGI Database |
|---|---|---|
| | GenBank and/or UniGene | Accession |
| Gene | Accession Number | Number |
| CHRM1 (Muscarinic | human: X15263, M35128 | MGI: 88396 |
| Ach M1) receptor | Y00508, X52068 | |
| CHRM2 (Muscarinic | human: M16404, AB041391, | |
| Ach M2) receptor | X15264 | |
| | mouse: AF264049 | |
| CHRM3 (Muscarinic | human: U29589, AB041395, | |
| Ach M3) receptor | X15266 | |
| | mouse: AF264050 | |
| CHRM4 (Muscarinic | human: X15265, M16405 | MGI: 88399 |
| Ach M4) receptor | | |
| CHRM5 (Muscarinic | human: AF026263, M80333 | |
| Ach M5) receptor | rat: NM_017362 | |
| | mouse: AI327507 | |
| CHRNA1 (nicotinic | human: Y00762, X02502, | MGI: 87885 |
| alpha1) receptor | S77094 | |
| CHRNA2 (nicotinic | human: U62431, Y16281 | MGI: 87886 |
| alpha2) receptor | | |
| CHRNA3 (nicotinic | human: NM_000743, | |
| alpha3) receptor | U62432, M37981, M86383, | |
| | Y08418 | |
| CHRNA4 (nicotinic | human: U62433, L35901, | MGI: 87888 |
| alpha4) receptor | Y08421, X89745, X87629 | |
| CHRNA5 (nicotinic | human: U62434, Y08419, | MGI: 87889 |
| alpha5) receptor | M83712 | |
| CHRNA7 (nicotinic | human: X70297, Y08420, | MGI: 99779 |
| alpha7) receptor | Z23141, U40583, U62436, | |
| | L25827, AF036903 | |
| CHRNB1 (nicotinic | human: X14830 | MGI: 87890 |
| Beta 1) receptor | | |
| CHRNB2 (nicotinic | human: U62437, X53179, | MGI: 87891 |
| Beta 2) receptor | Y08415, AJ001935 | |
| CHRNB3 (nicotinic | human: Y08417, X67513, | |
| Beta 3) receptor | U62438, RIKEN BB284174 | |
| CHRNB4 (nicotinic | human: U48861, U62439, | MGI: 87892 |
| Beta 4) receptor | Y08416, X68275 | |
| CHRNG nicotinic | human: X01715, M11811 | MGI: 87895 |
| gamma immature | | |
| muscle receptor | | |
| CHRNE nicotinic | human: X66403 | |
| epsilon receptor | mouse: NM_009603 | |
| CHRND nicotinic | human: X55019 | MGI: 87893 |
| delta receptor | | |

TABLE 3

| | | MGI Database |
|---|---|---|
| | GenBank and/or UniGene | Accession |
| Gene | Accession Number | Number |
| th (tyrosine | human: M17589 | MGI: 98735 |
| hydroxylase) | | |
| dat (dopamine | human: NM_001044 | MGI: 94862 |
| transporter) | | |
| dopamine receptor 1 | human UniGene: X58987, | MGI: 99578 |
| | S58541, X55760, X55758 | |
| dopamine receptor 2 | human UniGene: X51362, | MGI: 94924 |
| | M29066, AF050737, S62137, | |
| | X51645, M30625, S69899 | |
| dopamine receptor 3 | human UniGene: U25441, | MGI: 94925 |
| | U32499 | |
| dopamine receptor 4 | human UniGene: L12398, | MGI: 94926 |
| | S76942 | |
| dopamine receptor 5 | human UniGene: M67439, | MGI: 94927 |
| | M67439, X58454 | |
| dbh | human UniGene: X13255 | MGI: 94864 |
| dopamine beta | | |
| hydroxylase | | |

TABLE 4

| | | MGI Database |
|---|---|---|
| | GenBank and/or UniGene | Accession |
| Gene | Accession Number | Number |
| GABA A A2 | human: S62907 | MGI: 95614 |
| GABRA2 | | |
| GABA receptor A2 | | |
| GABA A A3 | human: S62908 | MGI: 95615 |
| GABRA3 | | |
| GABA receptor A3 | | |
| GABA A A4 | human: NM_000809, U30461 | MGI: 95616 |
| GABRB4 | | |
| GABA receptor A4 | | |
| GABA A A5 | human: NM_000810, | |
| GABRB5 | L08485, AF061785, | |
| GABA receptor A5 | AF061785, AF061785 | |
| GABA A A6 | human: S81944, AF053072 | MGI: 95618 |
| GABRB6 | | |
| GABA receptor A6 | | |
| GABA B1 | human: X14767, M59216 | MGI: 95619 |
| GABRB1 | | |
| GABA receptor B1 | | |
| GABA B2 | human: S67368, S77554, | |
| GABRB2 | S77553 | |
| GABA receptor B2 | mouse: MM4707 | |
| GABA B3 | human: M82919 | MGI: 95621 |
| GABRB3 | | |
| GABA receptor B3 | | |
| GABRG1 | | MGI: 103156 |
| GABA-A receptor, | | |
| gamma 1subunit | | |
| GABRG2 | human: X15376 | MGI: 95623 |
| GABA-A receptor, | | |
| gamma 2 subunit | | |
| GABRG3 | human: S82769 | |
| GABA-A receptor, | | |
| gamma 3 subunit | | |
| GABRD | human: AF016917 | MGI: 95622 |
| GABA-A receptor, | | |
| delta subunit | | |
| GABRE | human: U66661, Y07637, | |
| GABA-A receptor, | Y09765, U92283, Y09763, | |
| epsilon subunit | U92285 | |
| | mouse: NM_017369 | |
| GABA A pi | human: U95367, AF009702 | |
| GABRP | | |
| GABA-A receptor, | | |
| pi subunit | | |
| GABA A theta | mouse NM_020488 | |
| GABA receptor | | |
| theta | | |
| GABA R1a | human: M62400 | MGI: 95625 |
| GABA receptor | | |
| rho 1 GABRR1 | | |
| GABA receptor | | |
| rho 1 | | |
| GABA R2 | human: M86868 | MGI: 95626 |
| GABA receptor | | |
| a rho 2 | | |
| GABRR2 | | |
| GABA receptor | | |
| rho 2 | | |

TABLE 5

| | | MGI Database |
|---|---|---|
| | GenBank and/or UniGene | Accession |
| Gene | Accession Number | Number |
| GRIA1 | human: NM_000827, M64752, | |
| GluR1 | X58633 M81886 | |
| | mouse: NM_008165 | |
| GRIA2 | human: L20814 | |
| GlurR2 | rat: M85035 | |
| | mouse: AF250875 | |
| GRIA3 | human: U10301, X82068, U10302 | |
| GluR3 | rat: M85036 | |
| GRIA4 | human: U16129 | |
| GluR4 | rat: NM_017263 | |
| GRIK1 | human: L19058, U16125, | MGI: 95814 |
| glutamate ionotropic kainate 1 | AF107257, AF107259 | |
| gluR5 | | |
| GRIK2 | human: U16126 | |
| gluR6 | mouse: NM_010349, RIKEN | |
| | BB359097 | |
| GRIK3 | human: U16127 | |
| gluR7 | mouse: AF245444 | |
| GRIK4 | human: S67803 | MGI: 95817 |
| KA1 | | |
| GRIK5 | human: S40369 | MGI: 95818 |
| KA2 | | |
| GRIN1 | human: D13515, L05666, L13268, | MGI: 95819 |
| NR1nmdar1 | L13266, AF015731, AF015730, | |
| NMDA receptor 1 | U08106, L13267 | |
| GRIN2A | human: NM_000833, U09002, | |
| NR2A | U90277 | |
| NMDA receptor 2A | mouse: NM_008170 | |
| GRIN2B | human: NM_000834, U11287, | MGI: 95821 |
| NR2B | U90278, U88963 | |
| NMDA receptor 2B | | |
| GRIN2C | human: U77782, L76224 | MGI: 95822 |
| NR2C | | |
| NMDA receptor 2C | | |
| GRIN2D | human: U77783 | MGI: 95823 |
| NR2D | | |
| NMDA receptor 2D | | |
| GRM1 | human: NM_000838, L76627, | |
| mGluR 1a and 1b alternate | AL035698, U31215, AL035698, | |
| splicing type I | U31216, L76631 | |
| mGluR1a | mouse: BB275384, BB181459, | |
| | BB177876 | |
| GRM2 | human: L35318 | |
| mGluR 2 type II | Sheep: AF229842 | |
| mGluR2 | | |
| GRM3 | human: X77748 | |
| mGluR3 type II | mouse: AH008375; MM45836 | |
| mGluR3 | | |
| GRM4 | human: X80818 | |
| mGluR4 type III | | |
| mGluR4 | | |
| GRM5 | human: D28538, D28539 | |
| mGluR5a and 5b alt splice 32 | mouse: AF140349 | |
| residues | | |
| mGluR5 | | |
| GRM6 | human: NM_000843, U82083, | |
| mGluR6 type III | AJ245872, AJ245871 | |
| mGluR6 | rat: AJ245718 | |
| GRM7 | human: NM_000844, X94552 | |
| mGluR7 type III | mouse: RIKEN BB357072 | |
| mGluR7 | | |
| GRM8 | human: NM_000845, U95025, | |
| mGluR8 type III | AJ236921, AJ236922, AC000099 | |
| mGluR8 | mouse: U17252 | |
| GRID2 | human: AF009014 | MGI: 95813 |
| glut ionotropic delta | | |
| excitatory amino acid | human: U03505, U01824, Z32517, | MGI: 101931 |
| transporter2 | D85884 | |
| glutamate/aspartate transporter II | | |
| glutamate transporter GLT1 | | |
| glutamate transporter SLC1A2 | | |
| glial high affinity glutamate | | |
| transporter | | |
| EAAC1 | human: U08989, U03506, U06469 | MGI: 105083 |
| neural SLC1A1 | | |
| neuronal/epithelial high affinity | | |
| glutamate transporter | | |
| EEAT1 | human: D26443, AF070609, | MGI: 99917 |
| SLC1A3 | L19158, U03504, Z31713 | |
| glial high affinity glutamate | | |
| transporter | | |
| EAAT4 | human: U18244, AC004659 | MGI: 1096331 |
| neural SLC1A6 | | |
| high affinity aspartate/glutamate | | |
| transporter | | |

TABLE 6

| | | MGI Database |
|---|---|---|
| | GenBank and/or UniGene | Accession |
| Gene | Accession Number | Number |
| Glycine receptors | human: X52009 | MGI: 95747 |
| alpha 1 | | |
| GLRA1 | | |
| Glycine receptors | human: X52008, AF053495 | MGI: 95748 |
| alpha 2 | | |
| GLRA2 | | |
| Glycine receptors | human: AF017724, U93917, | |
| alpha 3 | AF018157 | |
| GLRA3 | mouse: AF214575 | |
| Glycine receptors | no human | |
| alpha 4 | mouse: X75850, X75851, | |
| GLRA4 | X75852, X75853 | |
| glycine receptor beta | human: U33267, AF094754, | MGI: 95751 |
| GLRB | AF094755 | |

TABLE 7

| | | MGI Database |
|---|---|---|
| | GenBank and/or UniGene | Accession |
| Gene | Accession Number | Number |
| Histamine | human: Z34897, D28481, X76786, | MGI: 107619 |
| H1-receptor 1 | AB041380, D14436, AF026261 | |
| Histamine | human: M64799, AB023486, | MGI: 108482 |
| H2-receptor 2 | AB041384 | |
| Histamine | human: NM_007232 | |
| H3-receptor 3 | mouse: MM31751 | |

TABLE 8

| | | MGI Database |
|---|---|---|
| | GenBank and/or UniGene | Accession |
| Gene | Accession Number | Number |
| orexin OX-A | human: AF041240 | MGI: 1202306 |
| hypocretin 1 | | |
| Orexin B | | |
| Orexin receptor OX1R | human: AF041243 | |
| HCRTR1 | | |
| Orexin receptor OX2R | human: AF041245 | |
| HCRTR2 | | |
| leptinR-long | human: U66497, U43168, U59263, | MGI: 104993 |
| Leptin receptor long form | U66495, U52913, U66496, | |
| | U52914, U52912, U50748, | |
| | AK001042 | |
| MCH | human: M57703, S63697 | |
| melanin concentrating hormone | | |
| PMCH | | |
| MC3R | human: GDB: 138780 | MGI: 96929 |
| MC3 receptor | mouse: MM57183 | |
| melanocortin 3 receptor | | |
| MC4R | human: S77415, L08603, | |
| MC4 receptor | NM_005912 | |
| melanocortin 4 receptor | | |
| MC5R | human: L27080, Z25470, U08353 | MGI: 99420 |
| MC5 receptor | | |
| melanocortin 5 receptor | | |
| prepro-CRF | human: V00571 | |
| corticotropin-releasing factor | rat: X03036, M54987 | |
| precursor | | |
| CRH | | |
| corticotropin releasing hormone | | |
| CRHR1 | human: L23332, X72304, L23333, | MGI: 88498 |
| CRH/CRF receptor 1 | AF039523, U16273 | |
| CRF R2 | human: U34587, AF019381, | MGI: 894312 |
| CRH/CRF receptor 2 | AF011406, AC004976, AC004976 | |
| CRHBP | human: X58022, S60697 | MGI: 88497 |
| CRF binding protein | | |
| Urocortin | human: AF038633 | MGI: 1276123 |
| POMC | human: V01510, M38297, J00292, | MGI: 97742 |
| Pro-opiomelanocortin | M28636 | |
| CART | human: U20325, U16826 | MGI: 1351330 |
| cocaine and amphetamine | | |
| regulated transcript | | |
| NPY | human: K01911, M15789, | MGI: 97374 |
| Neuropeptide Y | M14298, AC004485 | |
| prepro NPY | | |
| NPY1R | human: M88461, M84755, | MGI: 104963 |
| NPY Y1 receptor | NM_000909 | |
| Neuropeptide Y1 receptor | | |
| NPY2R | human: U42766, U50146, U32500, | MGI: 108418 |
| NPY Y2 receptor | U36269, U42389, U76254, | |
| Neuropeptide Y2 receptor | NM_000910 | |
| NPY Y4 receptor | human: Z66526, U35232, U42387 | MGI: 105374 |
| Npy4R Neuropeptide Y4 receptor | | |
| (mouse) | | |
| NPY Y5 receptor | human: U94320, U56079, U66275 | MGI: 108082 |
| Npy5R Neuropeptide Y5 receptor | mouse: MM10685 | |
| (mouse) | | |
| NPY Y6 receptor | human: D86519, U59431, U67780 | MGI: 1098590 |
| Npy6r Neuropeptide Y receptor | | |
| (mouse) | | |
| CCK | human: NM_000729, L00354 | MGI: 88297 |
| cholecystokinin | | |
| CCKa receptor | human: L19315, D85606, L13605 | MGI: 99478 |
| CCKAR cholecystokinin receptor | U23430 | |
| CCKb receptor | human: D13305, L04473, L08112, | MGI: 99479 |
| CCKBR cholecystokinin receptor | L07746, L10822, D21219, | |
| | S70057, AF074029 | |
| AGRP | human: NM_001138, U88063, | MGI: 892013 |
| agouti related peptide | U89485 | |
| Galanin | human: M77140, L11144 | MGI: 95637 |
| GALP | | |
| Galanin like peptide | | |
| See, Jureus et al., 2000, | | |
| Endocrinology 141(7): 2703-06. | | |
| GalR1 receptor | human: NM_001480, U53511, | MGI: 1096364 |
| GALNR1 | L34339, U23854 | |
| galanin receptor1 | | |
| GalR2 receptor | human: AF040630, AF080586, | MGI: 1337018 |
| GALNR2 | AF042782 | |
| galanin receptor2 | | |
| GalR3 receptor | human: AF073799, Z97630, | MGI: 1329003 |
| GALNR3 | AF067733 | |
| Galr3 | | |
| galanin receptor3 | | |
| UTS2 | human: Z98884, AF104118 | MGI: 1346329 |
| prepro-urotensin II | | |
| GPR14 | human: AI263529 | |
| Urotensin receptor | mouse: AI385474 | |
| SST | human: J00306 | MGI: 98326 |
| somatostatin | | |
| SSTR1 | human: M81829 | MGI: 98327 |
| somatostatin receptor sst1 | | |
| SSTR2 | human: AF184174 M81830 | MGI: 98328 |
| somatostatin receptor sst2 | AF184174 | |
| SSTR3 | human: M96738, Z82188 | MGI: 98329 |
| somatostatin receptor sst3 | | |
| SSTR4 | human: L14856, L07833, D16826, | MGI: 105372 |
| somatostatin receptor sst4 | AL049651 | |
| SSTR5somatostatin receptor sst5 | human: D16827, L14865, | MGI: 894282 |
| | AL031713 | |
| GPR7 | human: U22491 | MGI: 891989 |
| G protein-coupled receptor 7 | | |
| opioid-somatostatin-like receptor | | |
| GPR8 | human: U22492 | |
| G protein-coupled receptor 8 | | |
| opioid-somatostatin-like receptor | | |
| PENK (pre Pro Enkephalin) | human: V00510, J00123 | MGI: 104629 |
| PDYN (Pre pro Dynorphin) | human: K02268, AL034562, | MGI: 97535 |
| | X00176 | |
| OPRM1 | human: L25119, L29301, U12569, | MGI: 97441 |
| μ opiate receptor | AL132774 | |
| OPRK1 | human: U11053, L37362, U17298 | MGI: 97439 |
| k opiate receptor | | |
| OPRD1 | human: U07882, U10504, | MGI: 97438 |
| delta opiate receptor | AL009181 | |
| OPRL1 | human: X77130, U30185 | MGI: 97440 |
| ORL1 opioid receptor-like | | |
| receptor | | |
| VR1 | human: NM_018727, BE466577 | |
| Vanilloid receptor subtype 1 | mouse: BE623398, | |
| VRL-1 | human: NM_015930 | MGI: 1341836 |
| vanilloid receptor-like protein 1 | rat: AB040873 | |
| VR1L1 | mouse: NM_011706 | |
| vanilloid receptor type 1 like | | |
| protein 1 VRL1 | | |
| vanilloid receptor-like protein 1 | | |
| VR-OAC | human: AC007834 | |
| vanilloid receptor-related | | |
| osmotically activated channel | | |
| CNR1 | human: U73304, X81120, X81120, | MGI: 104615 |
| cannaboid receptors CB1 | X54937, X81121 | |
| EDN1 | human: J05008, Y00749, S56805, | MGI: 95283 |
| endothelin 1 ET-1 | Z98050, M25380 | |
| GHRH | human: L00137, AL031659, | MGI: 95709 |
| growth hormone releasing | L00137 | |
| hormone | | |
| GHRHR | human: AF029342, U34195, | |
| growth hormone releasing | mouse: NM_010285 | |
| hormone receptor | | |
| PNOC | human: X97370, U48263, X97367 | MGI: 105308 |
| nociceptin orphanin FQ/nocistatin | | |
| NPFF | human: AF005271 | |
| neuropeptide FF precursor | mouse: RIKEN BB365815 | |
| neuropeptide FF receptor | human: AF257210, NM_004885, | |
| neuropeptide AF receptor | AF119815 | |
| G-protein coupled receptor | | |
| HLWAR77 | | |
| G-protein coupled receptor | | |
| NPGPR | | |
| GRP | human: K02054, S67384, S73265, | MGI: 95833 |
| gastrin releasing peptide | M12512 | |
| preprogastrin-releasing peptide | | |
| GRPR | human: M73481, U57365 | MGI: 95836 |
| gastrin releasing peptide receptor | | |
| BB2 | | |
| NMB | human: M21551 | |
| neuromedin B | mouse: AI327379 | |
| NMBR | human: M73482 | MGI: 1100525 |
| neuromedin B receptor BB1 | | |
| BRS3 | human: Z97632, L08893, X76498 | |
| bombesin like receptor subtype-3 | mouse: AB010280 | |
| uterine bombesin receptor | | |
| GCG PROglucagon | human: J04040, X03991, V01515 | MGI: 95674 |
| GLP-1 | | |
| GLP-2 | | |
| GCGR | human: U03469, L20316 | MGI: 99572 |
| glucagon receptor | | |
| GLP1R | human: AL035690, U01104, | MGI: 99571 |
| GLP1 receptor | U01157, L23503, U01156, | |
| | U10037 | |
| GLP2R | human: AF105367 | |
| GLP2 receptor | mouse; AF166265 | |
| VIP | human: M36634, M54930, | MGI: 98933 |
| vasoactive intestinal peptide | M14623, M33027, M11554, | |
| | L00158, M36612 | |
| SCT | mouse: NM_011328, X73580 | |
| secretin | | |
| PPYR1 | human: Z66526, U35232, U42387 | MGI: 105374 |
| pancreatic polypeptide receptor 1 | | |
| OXT | human: M25650, M11186, | |
| pre pro Oxytocin | X03173 | |
| | mouse: NM_011025, M88355 | |
| OXTR | human: X64878 | MGI: 109147 |
| OTR | | |
| oxytocin receptor | | |
| AVP | human: M25647, X03172, | MGI: 88121 |
| Preprovasopressin | M11166, AF031476, X62890, | |
| | X62891 | |
| AVPR1A | human: U19906, L25615, S73899, | |
| V1a receptor | AF030625, AF101725 | |
| vasopressin receptor1a | mouse: NM_016847 | |
| AVPR1B | human: D31833, L37112, | |
| V1b receptor | AF030512, AF101726 | |
| vasopressin receptor1b | mouse: NM_011924 | |
| AVPR2 | human: Z11687, U04357, L22206, | MGI: 88123 |
| V2 receptor | U52112, AF030626, AF032388, | |
| vasopressin receptor2 | AF101727, AF101728 | |
| NTS | human: NM_006183, U91618 | |
| proneurotensin/proneuromedin N | mouse: MM64201 | |
| Neurotensin tridecapeptide plus | | |
| neuromedin N | | |
| NTSR1 | human: X70070 | MGI: 97386 |
| Neurotensin receptor NT1 | | |
| NTSR2 | human: Y10148 | |
| Neurotensin receptor NT2 | mouse: NM_008747 | |
| SORT1 | human: X98248, L10377 | MGI: 1338015 |
| sortilin 1 neurotensin receptor 3 | | |
| BDKRB1 | human: U12512, U48231, U22346, | MGI: 88144 |
| Bradykinin receptor 1 | AJ238044, AF117819 | |
| BDKRB2 | human: X69680, S45489, S56772, | MGI: 102845 |
| Bradykinin receptor B2 | M88714, X86164, X86163, | |
| | X86165 | |
| GNRH1 | human: X01059, M12578, X15215 | MGI: 95789 |
| GnRH | | |
| gonadotrophin releasing hormone | | |
| GNRH2 | human: AF036329 | |
| GnRH | | |
| gonadotrophin releasing hormone | | |
| GNRHR | human: NM_000406, L07949, | MGI: 95790 |
| GnRH | S60587, L03380, S77472, Z81148, | |
| gonadotrophin releasing hormone | U19602 | |
| receptor | | |
| CALCB | human: X02404, X04861 | |
| calcitonin-related polypeptide, | | |
| beta | | |
| CALCA | human: M26095, X00356, | MGI: 88249 |
| calcitonin/calcitonin-related | X03662, M64486, M12667, | |
| polypeptide, alpha | X02330, X15943 | |
| CALCR | human: L00587 | MGI: 101950 |
| calcitonin receptor | | |
| TAC1 (also called tac2) | human: X54469, U37529, | MGI: 98474 |
| neurokinin A | AC004140 | |
| TAC3 | human: NM_013251 | |
| neurokinin B | rat: NM_017053 | |
| TACR2 | human: M75105, M57414, | |
| neurokinin a (subK) receptor | M60284 | |
| TACR1 | human: M84425, M74290, | MGI: 98475 |
| tachykinin receptor NK2 (Sub P | M81797, M76675, X65177, | |
| and K) | M84426 | |
| TACR3 | human: M89473 X65172 | |
| tachykinin receptor NK3 (Sub P | | |
| and K) neuromedin K | | |
| ADCYAP1 | human: X60435 | MGI: 105094 |
| PACAP | | |
| NPPA | human: M54951, X01470, | MGI: 97367 |
| atrial naturietic peptide (ANP) | AL021155, M30262, K02043, | |
| precursor | K02044 | |
| atrial natriuretic factor (ANF) | | |
| precursor | | |
| pronatriodilatin precursor | | |
| prepronatriodilatin | | |
| NPPB | human: M25296, AL021155, | |
| atrial naturietic peptide (BNP) | M31776 | |
| precursor | mouse: NM_008726 | |
| NPR1 | human: X15357, AB010491 | MGI: 97371 |
| naturietic peptide receptor 1 | | |
| NPR2 | human: L13436, AJ005282, | MGI: 97372 |
| naturietic peptide receptor 2 | AB005647 | |
| NPR3 | human: M59305, AF025998, | MGI: 97373 |
| naturietic peptide receptor 3 | X52282 | |
| VIPR1 | human: NM_004624, L13288, | MGI: 109272 |
| VPAC1 | X75299, X77777, L20295, | |
| VIP receptor 1 | U11087 | |
| VIPR2 | human: X95097, L36566, Y18423, | MGI: 107166 |
| VIP receptor 2 | L40764, AF027390 | |
| PACAP receptor | | |

TABLE 9

| | | MGI Database |
|---|---|---|
| | GenBank and/or UniGene | Accession |
| Gene | Accession Number | Number |
| 5HT1A | human: M83181, AB041403, | MGI: 96273 |
| serotonin | M28269, X13556 | |
| receptor 1A | | |
| 5HT2A | human: X57830 | MGI: 109521 |
| serotonin | | |
| receptor 2A | | |
| 5HT3 | human: AJ005205, D49394, S82612, | MGI: 96282 |
| serotonin | AJ005205, AJ003079, AJ005205, | |
| receptor 3 | AJ003080, AJ003078 | |
| 5HT1B | human: M81590, M81590, D10995, | MGI: 96274 |
| 5HT1Db | M83180, L09732, M75128, | |
| serotonin | AB041370, AB041377, AL049595 | |
| receptor 1B | | |
| 5HT1D alpha | human: AL049576 | MGI: 96276 |
| serotonin | | |
| receptor 1D | | |
| 5HT1E | human: NM_000865, M91467, | |
| serotonin | M92826, Z11166 | |
| receptor 1E | | |
| 5HT2B | human: NM_000867, X77307, | MGI: 109323 |
| serotonin | Z36748 | |
| receptor 2B | | |
| 5HT2C | human: NM_000868, U49516, | MGI: 96281 |
| serotonin | M81778, X80763, AF208053 | |
| receptor 2C | | |
| 5HT4 | human: Y10437, Y08756, Y09586, | |
| serotonin | Y13584, Y12505, Y12506, Y12507, | |
| receptor 4 | AJ011371, AJ243213 | |
| (has 5 subtypes | | |
| isoforms) | | |
| 5HT5A | human: X81411 | MGI: 96283 |
| serotonin | | |
| receptor 5A | | |
| 5Ht5B | rat: L10073 | |
| serotonin | | |
| receptor 5B | | |
| 5HT6 | human: L41147, AF007141 | |
| serotonin | | |
| receptor 6 | | |
| 5HT7 | human: U68488, U68487, L21195, | |
| serotonin | X98193 | |
| receptor 7 | mouse: MM8053 | |
| sert | human UniGene: L05568 | MGI: 96285 |
| serotonin | | |
| transporter | | |
| TPRH | human UniGene: AF057280, X52836, | MGI: 98796 |
| TPH (Tph) | L29306 | |
| tryptophan | | |
| hydroxylase | | |

TABLE 10

| | | MGI Database |
|---|---|---|
| | GenBank and/or UniGene | Accession |
| Gene | Accession Number | Number |
| P2RX1 | human: U45448, X83688, | MGI: 1098235 |
| P2x1 receptor | AF078925, AF020498 | |
| purinergic receptor P2X, | | |
| ligand-gated | | |
| ion channel | | |
| P2RX3 | human: Y07683 | |
| purinergic receptor P2X, | mouse: RIKEN BB459124, | |
| ligand-gated | RIKEN BB452419 | |
| ion channel, 3 | | |
| P2RX4 | human: U83993, Y07684, | MGI: 1338859 |
| purinergic receptor P2X, | U87270, AF000234 | |
| ligand-gated | | |
| ion channel, 4 | | |
| P2RX5 | human: AF168787, | |
| purinergic receptor P2X, | AF016709, U49395, U49396, | |
| ligand-gated | AF168787 | |
| ion channel, 5 | rat: AF070573 | |
| P2RXL1 | human UniGene: AB002058 | MGI: 1337113 |
| purinergic receptor | | |
| P2X-like 1, | | |
| orphan receptor | | |
| P2RX6 | | |
| P2RX7 | human: Y09561, Y12851 | MGI: 1339957 |
| purinergic receptor P2X, | | |
| ligand-gated | | |
| ion channel, 7 | | |
| P2RY1 | human: Z49205 | MGI: 105049 |
| purinergic receptor P2Y, | | |
| G-protein | | |
| coupled 1 | | |
| P2RY2 | human: U07225 S74902 | |
| purinergic receptor P2Y, | rat: U56839 | |
| G-protein | | |
| coupled, 2 | | |
| P2RY4 pyrimidinergic | human: X91852, X96597, | |
| receptor P2Y, | U40223 | |
| G-protein coupled, 4 | | |
| P2RY6 | human: X97058, U52464, | |
| pyrimidinergic | AF007892, AF007891, | |
| receptor P2Y, G- | AF007893 | |
| protein coupled, 6 | | |
| P2RY11 | human: AF030335 | |
| purinergic receptor | | |
| P2Y, G-protein | | |
| coupled, 11 | | |

TABLE 11

| | GenBank and/or UniGene | MGI Database Accession |
|---|---|---|
| Gene | Accession Number | Number |
| SCN1A | human: X65362 | MGI: 98246 |
| sodium channel, voltage-gated, | | |
| type I, alpha | | |
| SCN1B | human: L16242, L10338, U12194, | MGI: 98247 |
| sodium channel, voltage-gated, | NM_001037 | |
| type I, beta | | |
| SCN2B | human: AF049498, AF049497, | MGI: 106921 |
| sodium channel, voltage-gated, | AF007783 | |
| type II, beta | | |
| SCN5A | human: M77235 | |
| sodium channel, voltage-gated, | | |
| type V, alpha | | |
| SCN2A1 | | MGI: 98248 |
| sodium channel, voltage-gated, | | |
| type II, alpha 1 | | |
| SCN2A2 | human: M94055, X65361, M91803 | |
| sodium channel, voltage-gated, | | |
| type II, alpha 2 | | |
| SCN3A | human: AB037777, AJ251507 | MGI: 98249 |
| sodium channel, voltage-gated, | | |
| type III, alpha | | |
| SCN4A | human: M81758, L01983, L04236, | MGI: 98250 |
| sodium channel, voltage-gated, | U24693 | |
| type IV, alpha | | |
| SCN6A | human: M91556 | |
| sodium channel, voltage-gated, | | |
| type VII or VI | | |
| SCN8a | human: AF225988, AB027567 | MGI: 103169 |
| SCN8A sodium channel, | | |
| voltage-gated, type VIII | | |
| SCN9A | human: X82835, RIKEN BB468679 | |
| sodium channel, voltage-gated, | mouse: MM40146 | |
| type IX, alpha | | |
| SCN10A | human: NM_006514, AF117907 | |
| sodium channel, voltage-gated, | | |
| type X, | | |
| SCN11A | human: AF188679 | MGI: 1345149 |
| sodium channel, voltage-gated, | | |
| type XI, alpha | | |
| SCN12A | human: NM_014139 | |
| sodium channel, voltage-gated, | | |
| type XII, alpha | | |
| SCNN1A | human: X76180, Z92978, L29007, | MGI: 101782 |
| sodium channel, nonvoltage- | U81961, U81961, U81961, U81961, | |
| gated 1 alpha | U81961 | |
| SCN4B | | |
| sodium channel, voltage-gated, | | |
| type IV, beta | | |
| SCNN1B | human: X87159, L36593, | |
| sodium channel, nonvoltage- | AJ005383, AC002300, U16023 | |
| gated 1, beta | | |
| SCNN1D | human: U38254 | |
| sodium channel, nonvoltage- | | |
| gated 1, delta | | |
| SCNN1G | human: X87160, L36592, U35630 | MGI: 104695 |
| sodium channel, nonvoltage- | | |
| gated 1, gamma | | |
| CLCN1 | human: Z25884, Z25587, M97820, | MGI: 88417 |
| chloride channel 1, skeletal | Z25753 | |
| muscle | | |
| CLCN2 | human: AF026004 | MGI: 105061 |
| chloride channel 2 | | |
| CLCN3 | human: X78520, AL117599, | MGI: 103555 |
| chloride channel 3 | AF029346 | |
| CIC3 | | |
| CLCN4 | human: AB019432 X77197 | MGI: 104567 |
| chloride channel 4 | | |
| CLCN5 | human: X91906, X81836 | MGI: 99486 |
| chloride channel 5 | | |
| CLCN6 | human: D28475, X83378, | MGI: 1347049 |
| chloride channel 6 | AL021155, X99473, X99474, | |
| | X96391, AL021155, AL021155, | |
| | X99475, AL021155 | |
| CLCN7 | human: AL031600, U88844, | MGI: 1347048 |
| chloride channel 7 | Z67743, AJ001910 | |
| CLIC1 | human: X87689, AJ012008, | |
| chloride intracellular channel 1 | X87689, U93205, AF129756 | |
| CLIC2 | human: NM_001289 | |
| chloride intracellular channel 2 | | |
| CLIC3 | human: AF102166 | |
| chloride intracellular channel 3 | | |
| CLIC5 | human: AW816405 | |
| chloride intracellular channel 5 | | |
| CLCNKB | human: Z30644, S80315, U93879 | |
| chloride channel Kb | | |
| CLCNKA | human: Z30643, U93878 | MGI: 1329026 |
| chloride channel Ka | | |
| CLCA1 | human: AF039400, AF039401 | MGI: 1316732 |
| chloride channel, calcium | | |
| activated, family member 1 | | |
| CLCA2 | human: AB026833 | |
| chloride channel, calcium | | |
| activated, family member 2 | | |
| CLCA3 | human: NM_004921 | |
| chloride channel, calcium | | |
| activated, family member 3 | | |
| CLCA4 | human: AK000072 | |
| chloride channel, calcium | | |
| activated, family member 4 | | |
| KCNA1 kv1.1 | human: L02750 | MGI: 96654 |
| potassium voltage-gated | | |
| channel, shaker-related | | |
| subfamily, member 1 | | |
| KCNA2 | human: Hs.248139, L02752 | MGI: 96659 |
| potassium voltage-gated | mouse: MM56930 | |
| channel, shaker-related | | |
| subfamily, member 2 | | |
| KCNA3 | human: M85217, L23499, M38217, | MGI: 96660 |
| potassium voltage-gated | M55515 | |
| channel, shaker-related | | |
| subfamily, member 3 | | |
| KCNA4 | human: M55514, M60450, L02751 | MGI: 96661 |
| potassium voltage-gated | | |
| channel, shaker-related | | |
| subfamily, member 4 | | |
| KCNA4L | | |
| potassium voltage-gated | | |
| channel, shaker-related | | |
| subfamily, member 4-like | | |
| KCNA5 | human: Hs.150208, M55513, | MGI: 96662 |
| potassium voltage-gated | M83254, M60451, M55513 | |
| channel, shaker-related | mouse: MM1241 | |
| subfamily, member 5 | | |
| KCNA6 | human: X17622 | MGI: 96663 |
| potassium voltage-gated | | |
| channel, shaker-related | | |
| subfamily, member 6 | | |
| KCNA7 | | MGI: 96664 |
| potassium voltage-gated | | |
| channel, shaker-related | | |
| subfamily, member 7 | | |
| KCNA10 | human: U96110 | |
| potassium voltage-gated | | |
| channel, shaker-related | | |
| subfamily, member 10 | | |
| KCNB1 | human: L02840, L02840, X68302, | MGI: 96666 |
| potassium voltage-gated | AF026005 | |
| channel, Shab-related | | |
| subfamily, member 1 | | |
| KCNB2 | human: Hs.121498, U69962 | |
| potassium voltage-gated | mouse: MM154372 | |
| channel, Shab-related | | |
| subfamily, member 2 | | |
| KCNC1 | human: L00621, S56770 | MGI: 96667 |
| potassium voltage-gated | | |
| channel, Shaw-related | | |
| subfamily, member 1 | | |
| KCNC2 | | MGI: 96668 |
| potassium voltage-gated | | |
| channel, Shaw-related | | |
| subfamily, member 2 | | |
| KCNC3 | human: AF055989 | MGI: 96669 |
| potassium voltage-gated | | |
| channel, Shaw-related | | |
| subfamily, member 3 | | |
| KCNC4 | human: M64676 | MGI: 96670 |
| potassium voltage-gated | | |
| channel, Shaw-related | | |
| subfamily, member 4 | | |
| KCND1 | human: AJ005898, AF166003 | MGI: 96671 |
| potassium voltage-gated | | |
| channel, Shal-related family, | | |
| member 1 | | |
| KCND2 | human: AB028967, AJ010969, | |
| potassium voltage-gated | AC004888 | |
| channel, Shal-related subfamily, | | |
| member 2 | | |
| KCND3 | human: AF120491, AF048713, | |
| potassium voltage-gated | AF048712, AL049557 | |
| channel, Shal-related subfamily, | | |
| member 3 | | |
| KCNE1 | mouse: NM_008424 | |
| potassium voltage-gated | | |
| channel, Isk-related family, | | |
| member 1 | | |
| KCNE1L | human: AJ012743, NM_012282 | |
| potassium voltage-gated | | |
| channel, Isk-related family, | | |
| member 1-like | | |
| KCNE2 | human: AF302095 | |
| potassium voltage-gated | | |
| channel, Isk-related family, | | |
| member 2 | | |
| KCNE3 | human: NM_005472, | |
| potassium voltage-gated | rat: AJ271742 | |
| channel, Isk-related family, | mouse: MM18733 | |
| member 3 | | |
| KCNE4 | mouse: MM24386 | |
| potassium voltage-gated | | |
| channel, Isk-related family, | | |
| member 4 | | |
| KCNF1 | human: AF033382 | |
| potassium voltage-gated | | |
| channel, subfamily F, member 1 | | |
| KCNG1 | human: AF033383, AL050404 | |
| potassium voltage-gated | | |
| channel, subfamily G, member 1 | | |
| KCNG2 | human: NM_012283 | |
| potassium voltage-gated | | |
| channel, subfamily G, member 2 | | |
| KCNH1 | human: AJ001366, AF078741, | |
| potassium voltage-gated | AF078742 | |
| channel, subfamily H (eag- | mouse: NM_010600 | |
| related), member 1 | | |
| KCNH2 | human: U04270, AJ010538, | MGI: 1341722 |
| potassium voltage-gated | AB009071, AF052728 | |
| channel, subfamily H (eag- | | |
| related), member 2 | | |
| KCNH3 | human: AB022696, AB033108, | |
| potassium voltage-gated | Hs.64064 | |
| channel, subfamily H (eag- | mouse: NM_010601, MM100209 | |
| related), member 3 | | |
| KCNH4 | human: AB022698 | |
| potassium voltage-gated | rat: BEC2 | |
| channel, subfamily H (eag- | | |
| related), member 4 | | |
| KCNH5 | human: Hs.27043 | |
| potassium voltage-gated | mouse: MM44465 | |
| channel, subfamily H (eag- | | |
| related), member 5 | | |
| KCNJ1 | human: U03884, U12541, U12542, | |
| potassium inwardly-rectifying | U12543 | |
| channel, subfamily J, member 1 | rat: NM_017023 | |
| KCNJ2 | human: U16861, U12507, U24055, | MGI: 104744 |
| potassium inwardly-rectifying | AF011904, U22413, AF021139 | |
| channel, subfamily J, member 2 | | |
| KCNJ3 | human: U50964 U39196 | |
| potassium inwardly-rectifying | mouse: NM_008426 | |
| channel, subfamily J, member 3 | | |
| KCNJ4 | human: Hs.32505, U07364, Z97056, | MGI: 104743 |
| potassium inwardly-rectifying | U24056, Z97056 | |
| channel, subfamily J, member 4 | mouse: MM104760 | |
| KCNJ5 | human: NM_000890 | MGI: 104755 |
| potassium inwardly-rectifying | | |
| channel, subfamily J, member 5 | | |
| KCNJ6 | human: Hs.11173, U52153, D87327, | |
| potassium inwardly-rectifying | L78480, S78685, AJ001894 | |
| channel, subfamily J, member 6 | mouse: NM_010606, MM4276 | |
| | rat: NM_013192 | |
| KCNJ8 | human: D50315, D50312 | MGI: 1100508 |
| potassium inwardly-rectifying | | |
| channel, subfamily J, member 8 | | |
| KCNJ9 | human: U52152 | MGI: 108007 |
| potassium inwardly-rectifying | | |
| channel, subfamily J, member 9 | | |
| KCNJ10 | human: Hs.66727, U52155, U73192, | MGI: 1194504 |
| potassium inwardly-rectifying | U73193 | |
| channel, subfamily J, member | | |
| 10 | | |
| KCNJ11 | human: Hs.248141, D50582 | MGI: 107501 |
| potassium inwardly-rectifying | mouse: MM4722 | |
| channel, subfamily J, member | | |
| 11 | | |
| KCNJ12 | human: AF005214, L36069 | MGI: 108495 |
| potassium inwardly-rectifying | | |
| channel, subfamily J, member | | |
| 12 | | |
| KCNJ13 | | |
| potassium inwardly-rectifying | human: AJ007557, AB013889, | |
| channel, subfamily J, member | AF061118, AJ006128, AF082182 | |
| 13 | rat: AB034241, AB013890, | |
| | AB034242 | |
| | guinea pig: AF200714 | |
| KCNJ14 | | |
| potassium inwardly-rectifying | human: Hs.278677 | |
| channel, subfamily J, member | mouse: Kir2.4, MM68170 | |
| 14 | | |
| KCNJ15 | human: Hs.17287, U73191, D87291, | |
| potassium inwardly-rectifying | Y10745 | |
| channel, subfamily J, member | mouse: AJ012368, kir4.2, MM44238 | |
| 15 | | |
| KCNJ16 | human: NM_018658, Kir5.1 | |
| potassium inwardly-rectifying | mouse: AB016197 | |
| channel, subfamily J, member 1 | | |
| KCNK1 | human: U76996, U33632, U90065 | MGI: 109322 |
| potassium channel, subfamily | | |
| K, member 1 (TWIK-1) | | |
| KCNK2 | human: AF004711, RIKEN | |
| potassium channel, subfamily | BB116025 | |
| K, member 2 (TREK-1) | | |
| KCNK3 | human: AF006823 | MGI: 1100509 |
| potassium channel, subfamily | | |
| K, member 3 (TASK) | | |
| KCNK4 | | |
| potassium inwardly-rectifying | human: AF247042, AL117564 | |
| channel, subfamily K, member 4 | mouse: NM_008431 | |
| KCNK5 | human: NM_003740, AK001897 | |
| potassium channel, subfamily | mouse: AF259395 | |
| K, member 5 (TASK-2) | | |
| KCNK6 | human: AK022344 | |
| potassium channel, subfamily | | |
| K, member 6 (TWIK-2) | | |
| KCNK7 | human: NM_005714 | MGI: 1341841 |
| potassium channel, subfamily | mouse: MM23020 | |
| K, member 7 | | |
| KCNK8 | mouse: NM_010609 | |
| potassium channel, subfamily | | |
| K, member 8 | | |
| KCNK9 | human: AF212829 | |
| potassium channel, subfamily | guinea pig: AF212828 | |
| K, member 9 | | |
| KCNK10 | human: AF279890 | |
| potassium channel, subfamily | | |
| K, member 10 (TREK2) | | |
| KCNN1 | human: NM_002248, U69883 | |
| potassium intermediate/small | | |
| conductance calcium-activated | | |
| channel, subfamily N, member 1 | | |
| KCNN2 | mouse: MM63515 | |
| potassium intermediate/small | | |
| conductance calcium-activated | | |
| channel, subfamily member 2 | | |
| (hsk2) | | |
| KCNN4 | human: Hs.10082, AF022797, | MGI: 1277957 |
| potassium intermediate/small | AF033021, AF000972, AF022150 | |
| conductance calcium-activated | mouse: MM9911 | |
| channel, subfamily N, member 4 | | |
| KCNQ1 | human: U89364, AF000571, | MGI: 108083 |
| potassium voltage-gated | AF051426, AJ006345, AB015163, | |
| channel, KQT-like subfamily, | AB015163, AJ006345 | |
| member 1 | | |
| KCNQ2 | human: Y15065, D82346, | MGI: 1309503 |
| potassium voltage-gated | AF033348, AF074247, AF110020 | |
| channel, KQT-like subfamily, | | |
| member 2 | | |
| KCNQ3 | human: NM_004519, AF033347, | MGI: 1336181 |
| potassium voltage-gated | AF071491 | |
| channel, KQT-like subfamily, | | |
| member 3 | | |
| KCNQ4 | human: Hs.241376, AF105202, | |
| potassium voltage-gated | AF105216 | |
| channel, KQT-like subfamily, | mouse: AF249747 | |
| member 4 | | |
| KCNQ5 | human: NM_019842 | |
| potassium voltage-gated | | |
| channel, KQT-like subfamily, | | |
| member 5 | | |
| KCNS1 | human: AF043473 | |
| potassium voltage-gated | mouse: NM_008435 | |
| channel, delayed-rectifier, | | |
| subfamily S, member 1 | | |
| KCNS2 | mouse: NM_008436 | |
| potassium voltage-gated | | |
| channel, delayed-rectifier, | | |
| subfamily S, member 2 | | |
| KCNS3 | human: AF043472 | |
| potassium voltage-gated | | |
| channel, delayed-rectifier, | | |
| subfamily S, member 3 | | |
| KCNAB1 | L39833, U33428, L47665, X83127, | MGI: 109155 |
| potassium voltage-gated | U16953 | |
| channel, shaker-related | | |
| subfamily, beta member 1 | | |
| KCNAB2 | human: U33429, AF044253, | |
| potassium voltage-gated | AF029749 | |
| channel, shaker-related | mouse: NM_010598 | |
| subfamily, beta member 2 | | |
| KCNAB3 | human: NM_004732 | MGI: 1336208 |
| potassium voltage-gated | mouse: MM57241 | |
| channel, shaker-related | | |
| subfamily, beta member 3 | | |
| KCNJN1 | human: Hs.248143, U53143 | |
| potassium inwardly-rectifying | | |
| channel, subfamily J, inhibitor 1 | | |
| KCNMA1 | human: U11058, U13913, U11717, | MGI: 99923 |
| potassium large conductance | U23767, AF025999 | |
| calcium-activated channel, | | |
| subfamily M, alpha member 1 | | |
| kcnma3 | mouse: NM_008432 | |
| potassium large conductance | | |
| calcium-activated channel, | | |
| subfamily M, alpha member 3 | | |
| KCNMB1 | rat: NM_019273 | |
| potassium large conductance | | |
| calcium-activated channel, | | |
| subfamily M, beta member 1 | | |
| KCNMB2 | human: AF209747 | |
| potassium large conductance | mouse: NM_005832 | |
| calcium-activated channel, | | |
| subfamily M, beta member 2 | | |
| KCNMB3L | human: AP000365 | |
| potassium large conductance | | |
| calcium-activated channel, | | |
| subfamily M, beta member 3- | | |
| like | | |
| KCNMB3 | human: NM_014407, AF214561 | |
| potassium large conductance | | |
| calcium-activated channel | | |
| KCNMB4 | human: AJ271372, AF207992, | |
| potassium large conductance | RIKEN BB329438, RIKEN | |
| calcium-activated channel, sub | BB265233 | |
| M, beta 4 | | |
| HCN1 | | MGI: 1096392 |
| hyperpolarization activated | | |
| cyclic nucleotide-gated | | |
| potassium channel 1 | | |
| Cav1.1 α1 1.1 CACNA1S | human: L33798, U30707 | MGI: 88294 |
| calcium channel, voltage- | | |
| dependent, L type, alpha 1S | | |
| subunit | | |
| Cav1.2 α1 1.2 CACNA1C | human: Z34815, L29536, Z34822, | |
| calcium channel, voltage- | L29534, L04569, Z34817, Z34809, | |
| dependent, L type, alpha 1C | Z34813, Z34814, Z34820, Z34810, | |
| subunit | Z34811, L29529, Z34819, Z74996, | |
| | Z34812, Z34816, AJ224873, | |
| | Z34818, Z34821, AF070589, | |
| | Z26308, M92269 | |
| Cav1.3 α1 1.3 CACNA1D | human: M83566, M76558, D43747, | MGI: 88293 |
| calcium channel, voltage- | AF055575 | |
| dependent, L type, alpha 1D | | |
| subunit | | |
| Cav1.4 α1 1.4 CACNA1F | human: AJ224874, AF235097, | MGI: 1859639 |
| calcium channel, voltage- | AJ006216, AF067227, U93305 | |
| dependent, L type, alpha 1F | | |
| subunit | | |
| Cav2.1 α1 2.1 CACNA1A P/Q | human: U79666, AF004883, | MGI: 109482 |
| type calcium channel, voltage- | AF004884, X99897, AB035727, | |
| dependent, P/Q type, alpha 1A | U79663, U79665, U79664, | |
| subunit | U79667, U79668, AF100774 | |
| Cav2.2 α1 2.2 CACNA1B | human: M94172, M94173, U76666 | MGI: 88296 |
| calcium channel, voltage- | | |
| dependent, L type, alpha 1B | | |
| subunit | | |
| Cav2.3 α1 2.3 CACNA1E | human: L29385, L29384, L27745 | MGI: 106217 |
| calcium channel, voltage- | | |
| dependent, alpha 1E subunit | | |
| Cav3.1 α1 3.1 CACNA1G | human: AB012043, AF190860, | MGI: 1201678 |
| calcium channel, voltage- | AF126966, AF227746, AF227744, | |
| dependent, alpha 1G subunit | AF134985, AF227745, AF227747, | |
| | AF126965, AF227749, AF134986, | |
| | AF227748, AF227751, AF227750, | |
| | AB032949, AF029228 | |
| Cav3.2 α1 3.2 CACNA1H | human: AF073931, AF051946, | |
| calcium channel, voltage- | AF070604 | |
| dependent, alpha 1H subunit | | |
| Cav3.3 α1 3.3 CACNA1I | human: AF142567, AL022319, | |
| calcium channel, voltage- | AF211189, AB032946 | |
| dependent, alpha 1I subunit | | |

TABLE 12

| | GenBank and/or UniGene | MGI Database Accession |
|---|---|---|
| Gene | Accession Number | Number |
| NES (nestin) | no human | MGI: 101784 |
| scip | human: L26494 | MGI: 101896 |

TABLE 13

| | | MGI |
|---|---|---|
| | GenBank and/ | Database |
| | or UniGene | Accession |
| Gene | Accession Number | Number |
| Shh (Sonic Hedgehog) | human: L38518 | MGI: 98297 |
| Smoothened Shh receptor | human: U84401, | |
| | AF114821 | MGI: 108075 |
| Patched Shh binding protein | human: NM_000264 | |
| | rat: AF079162 | |

TABLE 14

| | GenBank and/ | MGI Database |
|---|---|---|
| | or UniGene | Accession |
| Gene | Accession Number | Number |
| CALB1 (calbindin d28 K) | human: X06661, M19879, | MGI: 88248 |
| CALB2 (calretinin) | human: NM_001740, | MGI: 101914 |
| | X56667, X56668 | |
| PVALB (parvalbumin) | human: X63578, X63070, | MGI: 97821 |
| | Z82184, X52695, Z82184 | |

TABLE 15

| | GenBank and/or UniGene | MGI Database Accession |
|---|---|---|
| Gene | Accession Number | Number |
| NTRK2 (Trk B) | human: U12140, X75958, S76473, | MGI: 97384 |
| | S76474 | |
| GFRA1 (GFR alpha 1) | human: NM_005264, AF038420, | MGI: 1100842 |
| | AF038421, U97144, AF042080, | |
| | U95847, AF058999 | |
| GFRA2 (GFRalpha 2) | human: U97145, AF002700, U93703 | MGI: 1195462 |
| GFRA3 (GFRalpha 3) | human: AF051767 | MGI: 1201403 |
| trka | human: M23102, X03541, X04201, | MGI: 97383 |
| Neurotrophin receptor | X06704, X62947, M23102, X62947, | |
| | M23102, AB019488, M12128 | |
| trkc | human: U05012, U05012, S76475, | MGI: 97385 |
| Neurotrophin receptor | AJ224521, S76476, AF052184 | |
| ret | human: S80552 | MGI: 97902 |
| Neurotrophic factor receptor | | |

All of the sequences identified by the sequence database identifiers in Tables 1-15 are hereby incorporated by reference in their entireties.

In yet another aspect of the invention, the characterizing gene sequence is a promoter that directs tissue-specific expression of the system gene coding sequence to which it is operably linked. For example, expression of the system gene coding sequences may be controlled by any tissue-specific promoter/enhancer element known in the art. Promoters that may be used to control expression include, but are not limited to, the following animal transcriptional control regions that exhibit tissue specificity and that have been utilized in transgenic animals: elastase I gene control region, which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); enolase promoter, which is active in brain regions, including the striatum, cerebellum, CA1 region of the hippocampus, or deep layers of cerebral neocortex (Chen et al., 1998, Molecular Pharmacology 54(3): 495-503); insulin gene control region, which is active in pancreatic beta cells (Hanahan, 1985, Nature 315:115-22); immunoglobulin gene control region, which is active in lymphoid cells (Grosschedl et al., 1984, Cell 38:647-58; Adames et al., 1985, Nature 318:533-38; Alexander et al., 1987, Mol. Cell. Biol. 7:1436-44); mouse mammary tumor virus control region, which is active in testicular, breast, lymphoid and mast cells (Leder et al., 1986, Cell 45:485-95); albumin gene control region, which is active in liver (Pinkert et al., 1987, Genes and Devel. 1:268-76); alpha-fetoprotein gene control region which is active in liver (Krumlauf et al., 1985, Mol. Cell. Biol. 5:1639-48; Hammer et al., 1987, Science 235:53-58); alpha 1-antitrypsin gene control region, which is active in the liver (Kelsey et al., 1987, Genes and Devel. 1:161-71); β-globin gene control region, which is active in myeloid cells (Mogram et al., 1985, Nature 315:338-40: Kollias et al., 1986, Cell 46:89-94); myelin basic protein gene control region, which is active in oligodendrocyte cells in the brain (Readhead et al.., 1987, Cell 48:703-12); myosin light chain-2 gene control region, which is active in skeletal muscle (Sani, 1985, Nature 314:283-86); and gonadotropic releasing hormone gene control region which is active in the hypothalamus (Mason et al., 1986, Science 234:1372-78).

In other embodiments, the characterizing gene sequence is protein kinase C, gamma (GenBank Accession Number: Z1 5114 (human); MGI Database Accession Number: MGI:97597); fos (Unigene No. MM5043 (mouse)); TH-elastin; Pax7 (Mansouri, 1998, The role of Pax3 and Pax7 in development and cancer, Crit. Rev. Oncog. 9(2):141-9); Eph receptor (Mellitzer et al., 2000, Control of cell behaviour by signalling through Eph receptors and ephrins; Cur.r Opin. Neurobiol. 10(3):400-08; Suda et al., 2000, Hematopoiesis and angiogenesis, Int. J. Hematol. 71(2):99-107; Wilkinson, 2000, Eph receptors and ephrins: regulators of guidance and assembly, Int. Rev. Cytol. 196:177-244; Nakamoto, 2000, Eph receptors and ephrins, Int. J. Biochem. Cell Biol. 32(1):7-12; Tallquist et al., 1999, Growth factor signaling pathways in vascular development, Oncogene 18(55):7917-32); islet-1 (Bang et al., 1996, Regulation of vertebrate neural cell fate by transcription factors, Curr. Opin. Neurobiol. 6(1):25-32; Ericson et al., 1995, Sonic hedgehog: a common signal for ventral patterning along the rostrocaudal axis of the neural tube, J. Dev. Biol. 39(5):809-16; β-actin; thy-1 (Caroni, 1997, Overexpression of growth-associated proteins in the neurons of adult transgenic mice, J. Neurosci. Methods 71(1):3-9).

As discussed above in Section 4.2, the trangenes of the invention include all or a portion of the characterizing gene genomic sequence, preferably at least all or a portion of the upstream regulatory sequences of the characterizing gene genomic sequences are present in the transgene, and at a minimum, the characterizing gen sequences that direct expression of the system gene coding sequences in substantially the same pattern as the endogenous characterizing gene in the transgenic mouse or anatomical region or tissue thereof are present on the transgene.

In certain cases, genomic sequences and/or clones or other isolated nucleic acids containing the genomic sequences of the gene of interest are not available for the desired species, yet the genomic sequence of the counterpart from another species or all or a portion of the coding sequence (e.g., cDNA or EST sequences) for the same species or another species is available. It is routine in the art to obtain the genomic sequence for a gene when all or a portion of the coding sequence is known for example by hybridization of the cDNA or EST sequence or other probe derived therefrom to a genomic library to identify clones containing the corresponding genomic sequence. The identified clones may then be used to identify clones that map either 3′ or 5′ to the identified clones, for example, by hybridization to overlapping sequences present in the clones of a library and, by repeating the hybridization, “walking” to obtain clones containing the entire genomic sequence. As discussed above, it is preferable to use libraries prepared with vectors that can accommodate and that contain large inserts of genomic DNA (for example, at least 25 kb, 50 kb, 100 kb, 150 kb, 200 kb, or 300 kb) such that it likely that a clone can be identified that contains the entire genomic sequence of the characterizing gene or, at least, the upstream regulatory sequences of the characterizing gene (all or a portion of the regulatory sequences sufficient to direct expression in the same pattern as the endogenous characterizing gene). Cross-species hybridization may be carried out by methods routine in the art to identify a genomic sequence from all species when the genomic or cDNA sequence of the corresponding gene in another species is known.

As also discussed above, methods are known in the rat and described herein for identifying the regulatory sequences necessary to confer endogenous characterizing gene expression on the system gene coding sequences (see Section 4.2, supra, and Section 5, infra). In specific embodiments, the characterizing gene sequences are on BAC clones from a BAC mouse genomic library, for example, but not limited to the CITB (Research Genetics) or RPCI-23 (BACPAC Resources, Children's Hospital Oakland Research Institute, Oakland, Calif.) libraries, or any other BAC library.

4.2.2. System Gene Sequences

A “system gene” encodes a detectable or selectable marker such as a signal-producing protein, epitope, fluorescent or enzymatic marker, or inhibitor of cellular function or, in specific embodiments, encodes a protein product that specifically activates or represses expression of a detectable or selectable marker. The system gene sequences may code for any protein that allows cells expressing that protein to be detected or selected (or specifically activates or represses the expression of a protein that allows cells expressing that protein to be detected or selected). Preferably, the system gene product (and in certain embodiments, a marker turned on or repressed by the system gene product) is not present in any cells of the animal (or ancestor thereof) prior to its being made transgenic; in other embodiments, the system gene product (and, in certain embodiments, a marker turned on or repressed by the system gene product) is not present in a tissue in the animal (or ancestor thereof) prior to its being made transgenic, which tissue contains the subpopulation of cells to be isolated by virtue of the expression of the system gene coding sequences in the subpopulation and which can be cleanly dissected from any other tissues that may express the system gene product (and/or marker) in the animal (or ancestor thereof) prior to its being made transgenic.

In certain embodiments, the system gene product (and/or a marker turned on or repressed by the system gene product) is expressed in the animal or in tissues neighboring and/or containing the subpopulation of cells to be isolated prior to the animal (or ancestor thereof) being made transgenic but is expressed at much lower levels, e.g., 2-fold, 5-fold, 10-fold, 50-fold, 100-fold, 200-fold, 500-fold, 1000-fold lower levels, than the system gene product (or marker transactivated thereby), i.e., than expression driven by the transgene. In a specific embodiment, the system gene coding sequences encode a fusion protein comprising or consisting of all or a portion of the system gene product that confer the detectable or selectable property on the fusion protein, for example, where the system gene sequence is an epitope that is not detected elsewhere in the transgenic animal or that is not detected in or neighboring the tissue that contains the subpopulation of cells to be isolated. In a specific embodiment, the detectable or selectable marker is expressed everywhere in the transgenic animal except where the system gene is expressed, for example, where the system gene codes for a repressor that represses the expression of the detectable or selectable marker which is otherwise constitutively expressed (e.g., is under the regulatory control of the β-actin promoter (preferred for neural tissue) or CMV promoter). In one aspect of the invention, expression of the system gene coding sequences in a subpopulation of cells of the transgenic animal (or explanted tissue thereof or dissociated cells thereof) permits detection, isolation and/or selection of the subpopulation.

In specific embodiments, the system gene encodes a marker enzyme, such as lac Z or β-lactamase, a reporter or signal-producing protein such as luciferase or GFP, a ribozyme, RNA interference (RNAi), or a conditional transcriptional regulator such as a tet repressor.

In one embodiment, the system gene encodes a protein-containing epitope not normally detected in the tissue of interest by immunohistological techniques. For example, the system gene could encode CD4 (a protein normally expressed in the immune system) and be expressed and detected in non-immune cells.

In another embodiment, the system gene encodes a tract-tracing protein such as a lectin (e.g., wheat germ agglutinin (WGA)).

In another embodiment, the system gene encodes a toxin.

In certain embodiments, the system gene encodes an RNA product that is an inhibitor such as a ribozyme, anti-sense RNA or RNAi.

A system gene polypeptide, fragment, analog, or derivative may be expressed as a chimeric, or fusion, protein product (comprising a system gene encoded peptide Joined at its amino- or carboxy-terminus via a peptide bond to an amino acid sequence of a different protein). Sequences encoding such a chimeric product can be made by ligating the appropriate nucleotide sequences encoding the desired amino acid sequences to each other by methods known in the art, in the proper coding frame, and expressing the chimeric product as part of the transgene as discussed herein. In a specific embodiment, the chimeric gene comprises or consists of all or a portion of the characterizing gene coding sequence fused in frame to an epitope tag.

The system gene coding sequences can be present at a low gene dose, such as one copy of the system gene per cell. In other embodiments, at least two, three, five, seven, ten or more copies of the system gene coding sequences are present per cell, e.g., multiple copies of the system gene coding sequences are present in the same transgene or are present in one copy in the transgene and more than one transgene is present in the cell. In a specific embodiment in which BACs are used to generate and introduce the transgene into the animal, the gene dosage is one copy of the system gene per BAC and at least two, three, five, seven, ten or more copies of the BAC per cell. More then one copy of the system gene coding sequences may be necessary in some instances to achieve detectable or selectable levels of the marker gene. In cases where the transgene is present at high copy numbers or even in certain circumstances when it is present at one copy per cell, coding sequences other than the system gene coding sequences, for example, the characterizing gene coding sequence, if present, and/or any other protein coding sequences (for example, from other genes proximal to the characterizing gene in the genomic DNA) are inactiviated to avoid over- or mis-expression of these other gene products.

4.2.2.1. System Gene Sequences Encoding Marker Enzymes

A gene that encodes a marker enzyme (or a chimeric protein comprising a catalytic or active fragment of the enzyme) is preferably selected for use as a system gene. The marker enzyme is selected so that it produces a detectable signal when a particular chemical reaction is conducted. Such enzymatic markers are advantageous, particularly when used in vivo, because detection of enzymatic expression is highly accurate and sensitive. Preferably, a marker enzyme is selected that can be used in vivo, without the need to kill and/or fix cells in order to detect the marker or enzymatic activity of the marker.

In specific embodiments, the system gene encodes β-lactamase (e.g., GeneBLAzer™ Reporter System, Aurora Biosciences), E. coil β-galactosidase (lacZ, InvivoGen), human placental alkaline phosphatase (PLAP, InvivoGen) (Kam et al.,1985, Proc. Natl. Acad. Sci. USA 82: 8715-19), E. coli β-glucuronidase (gus, Sigma) (Jefferson et al., 1986, Proc. Natl. Acad. Sci 83:8447-8451) alkaline phosphatase, horseradish peroxidase, with β-lactamase being particularly preferred (Zlokamik et al., 1998, Science 279: 84-88; incorporated herein by reference in its entirety). In other embodiments, the system gene encodes a chemiluminscent enzyme marker such as luciferase (Danilov et al., 1989, Bacterial luciferase as a biosensor of biologically active compounds. Biotechnology, 11:39-78; Gould et al., 1988, Firefly luciferase as a tool in molecular and cell biology, Anal. Biochem. 175(1):5-13; Kricka, 1988, Clinical and biochemical applications of luciferases and luciferins, Anal. Biochem. 175(1):14-21; Welsh et al., 1997, Reporter gene expression for monitoring gene transfer, Curr. Opin. Biotechnol. 8(5):617-22; Contag et al., 2000, Use of reporter genes for optical measurements of neoplastic disease in vivo, Neoplasia 2(1-2):41-52; Himes et al., 2000, Assays for transcriptional activity based on the luciferase reporter gene, Methods Mol. Biol. 130:165-74; Naylor et al., 1999, Reporter gene technology: the future looks bright, Biochem. Pharmacol. 58(5):749-57, all of which are incorporated by reference in their entireties).

Cells expressing PLAP, an enzyme that resides on the outer surface of the cell membrane, can be labeled using the method of Gustincich etal. (1997, Neuron 18: 723-36; incorporated herein by reference in its entirety).

Cells expressing β-glucuronidase can be assayed using the method of Lorincz et al., 1996, Cytometry 24(4): 321-29, which is hereby incorporated by reference in its entirety.

5 4.2.2.2. System Gene Sequences Encoding Reporters Or Signal-Producing Proteins

The system gene can encode a marker that produces a detectable signal. In one aspect of the invention, the system gene encodes a reporter or signal-producing protein. In another embodiment, the system gene encodes a signal-producing protein that is used to monitor a physiological state.

In one embodiment, the reporter is a fluorescent protein such as green fluorescent protein (GFP), including particular mutant or engineered forms of GFP such as BFP, CFP and YFP (Aurora Biosciences) (see, e.g., Tsien et al., U.S. Pat. No. 6,124,128, issued Sep. 26, 2000, entitled Long Wavelength Engineered Fluorescent Proteins; incorporated herein by reference in its entirety), enhanced GFP (EGFP) and DsRed (Clontech), blue, cyan, green. yellow, and red fluorescent proteins (Clontech), rapidly degrading GFP-fusion proteins, (see, e.g., Li et al., U.S. Pat. No. 6,130,313 issued Oct. 10, 2000, entitled Rapidly Degrading GFP-Fusion Proteins; incorporated herein by reference in its entirety), and fluorescent proteins homologous to GFP, some of which have spectral characteristics different from GFP and emit at yellow and red wavelengths (Matz et al., 1999, Nat. Biotechnol. 17(10): 969-73; incorporated herein by reference in its entirety).

In a specific embodiment, the system gene encodes a red, green, yellow, or cyan fluorescent protein (an “XFP”), such as one of those disclosed in Feng et al. (2000, Neuron, 28: 41-51; incorporated herein by reference in its entirety).

In a specific embodiment, the system gene encodes E. coli β-glucuronidase (gus), and intracellular fluorescence is generated by activity of β-glucuronidase (Lorincz et al., 1996, Cytometry 24(4): 321-29; incorporated herein by reference in its entirety). In another specific embodiment, a fluorescence-activated cell sorter (FACS) is used to detect the activity of the E. coli β-glucuronidase (gus) gene (Lorincz et al., 1996, Cytometry 24(4): 321-29). When loaded with the Gus substrate fluorescein-di-beta-D-glucuronide (FDGlcu), individual mammalian cells expressing and translating gus mRNA liberate sufficient levels of intracellular fluorescein for quantitative analysis by flow cytometry. This assay can be used to FACS-sort viable cells based on Gus enzymatic activity (see Section 4.7, infra), and the efficacy of the assay can be measured independently by using a fluorometric lysate assay. In another specific embodiment, the intracellular fluorescence generated by the activity of both β-glucuronidase and E. coli β-galactosidase enzymes are detected by FACS independently. Because each enzyme has high specificity for its cognate substrate, each reporter gene can be measured by FACS independently.

In another embodiment, the system gene encodes a fusion protein of one or more different detectable or selectable markers and any other protein or fragment thereof. In particular embodiments, the fusion protein consists of or comprises two different detectable or selectable markers or epitopes, for example a lacZ-GFP fusion protein or GFP fused to an epitope not normally expressed in the cell of interest. Preferably, the markers or epitopes are not normally expressed in the transformed cell population or tissue of interest.

In another embodiment, the system gene encodes a “measurement protein” such as a protein that signals cell state, e.g., a protein that signals intracellular membrane voltage.

4.2.3. Conditional Transcriptional Regualation Systems

In certain embodiments, the system gene can be expressed conditionally by operably linking at least the coding region for the system gene to all or a portion of the regulatory sequences from the characterizing gene, and then operably linking the system gene coding sequences and characterizing gene sequences to an inducible or repressible transcriptional regulation system. Alternatively and preferably, the system gene itself encodes a conditional regulatory element which in turn induces or represses the expression of a detectable or selectable marker.

Transactivators in these inducible or repressible transcriptional regulation systems are designed to interact specifically with sequences engineered into the vector. Such systems include those regulated by tetracycline (“tet systems”), interferon, estrogen, ecdysone, Lac operator, progesterone antagonist RU486, and rapamycin (FK506) with tet systems being particularly preferred (see, e.g., Gingrich and Roder, 1998, Annu. Rev. Neurosci. 21: 377-405; incorporated herein by reference in its entirety). These drugs or hormones (or their analogs) act on modular transactivators composed of natural or mutant ligand binding domains and intrinsic or extrinsic DNA binding and transcriptional activation domains. In certain embodiments, expression of the detectable or selectable marker can be regulated by varying the concentration of the drug or hormone in medium in vitro or in the diet of the transgenic animal in vivo.

The inducible or repressible genetic system can restrict the expression of the detectable or selectable marker either temporally, spatially, or both temporally and spatially.

In a preferred embodiment, the control elements of the tetracycline-resistance operon of E. coli is used as an inducible or repressible transactivator or transcriptional regulation system (“tet system”) for conditional expression of the detectable or selectable marker. A tetracycline-controlled transactivator can require either the presence or absence of the antibiotic tetracycline, or one of its derivatives, e.g., doxycycline (dox), for binding to the tet operator of the tet system, and thus for the activation of the tet system promoter (Ptet). Such an inducible or repressible tet system is preferably used in a mammalian cell.

In a specific embodiment, a tetracycline-repressed regulatable system (TrRS) is used (Agha-Mohammadi and Lotze, 2000, J. Clin. Invest. 105(9): 1177-83; incorporated herein by reference in its entirety). This system exploits the specificity of the tet repressor (tetR) for the tet operator sequence (tetO), the sensitivity of tetR to tetracycline, and the activity of the potent herpes simplex virus transactivator (VP16) in eukaryotic cells. The TrRS uses a conditionally active chimeric tetracycline-repressed transactivator (tTA) created by fusing the COOH-terminal 127 amino acids of vision protein 16 (VP 16) to the COOH terminus of the tetR protein (which may be the system gene). In the absence of tetracycline, the tetR moiety of tTA binds with high affinity and specificity to a tetracycline-regulated promoter (tRP), a regulatory region comprising seven repeats of tetO placed upstream of a minimal human cytomegalovirus (CMV) promoter or β-actin promoter (β-actin is preferable for neural expression). Once bound to the tRP, the VP 16 moiety of tTA transactivates the detectable or selectable marker gene by promoting assembly of a transcriptional initiation complex. However, binding of tetracycline to tetR leads to a conformational change in tetR accompanied with loss of tetR affinity for tetO, allowing expression of the system gene to be silenced by administering tetracycline. Activity can be regulated over a range of orders of magnitude in response to tetracycline.

In another specific embodiment, a tetracycline-induced regulatable system is used to regulate expression of a detectable or selectable marker, e.g., the tetracycline transactivator (tTA) element of Gossen and Bujard (1992, Proc. Natl. Acad. Sci. USA 89: 5547-51; incorporated herein by reference in its entirety).

In another specific embodiment, the improved tTA system of Shockett et al. (1995, Proc. Natl. Acad. Sci. USA 92: 6522-26, incorporated herein by reference in its entirety) is used to drive expression of the marker. This improved tTA system places the tTA gene under control of the inducible promoter to which tTA binds, making expression of tTA itself inducible and autoregulatory.

In another embodiment, a reverse tetracycline-controlled transactivator, e.g., rtTA2 S-M2, is used. rtTA2 S-M2 transactivator has reduced basal activity in the absence doxycycline, increased stability in eukaryotic cells, and increased doxycycline sensitivity (Urlinger et al., 2000, Proc. Natl. Acad. Sci. USA 97(14): 7963-68; incorporated herein by reference in its entirety).

In another embodiment, the tet-repressible system described by Wells et al. (1999, Transgenic Res. 8(5): 371-81; incorporated herein by reference in its entirety) is used. In one aspect of the embodiment, a single plasmid Tet-repressible system is used. Preferably, a “mammalianized” TetR gene, rather than a wild-type TetR gene (tetR) is used (Wells et al., 1999, Transgenic Res. 8(5): 371-81).

In other embodiments, conditional expression of the detectable or selectable gene is regulated by using a recombinase system that is used to turn on or off system gene expression by recombination in the appropriate region of the genome in which the marker gene is inserted. Such a recombinase system (in which the system gene encodes the recombinase) can be used to turn on or off expression of a marker (for review of temporal genetic switches and “tissue scissors” using recombinases, see Hennighausen & Furth, 1999, Nature Biotechnol. 17: 1062-63). Exclusive recombination in a selected cell type may be mediated by use of a site-specific recombinase such as Cre, FLP-wild type (wt), FLP-L or FLPe. Recomnbination may be effected by any art-known method, e.g., the method of Doetschman et al. (1987, Nature 330: 576-78; incorporated herein by reference in its entirety); the method of Thomas et al., (1986, Cell 44: 419-28; incorporated herein by reference in its entirety); the Cre-loxP recombination system (Sternberg and Hamilton, 1981, J. Mol. Biol. 150: 467-86; Lakso el al., 1992, Proc. Natl. Acad. Sci. USA 89: 6232-36; which are incorporated herein by reference in their entireties); the FLP recombinase system of Saccharomyces cerevisiae (O'Gorman et al., 1991, Science 251: 1351-55); the Cre-loxP-tetracycline control switch (Gossen and Bujard, 1992, Proc. Natl. Acad. Sci. USA 89: 5547-51); and ligand-regulated recombinase system (Kellendonk et al., 1999, J. Mol. Biol. 285: 175-82; incorporated herein by reference in its entirety). Preferably, the recombinase is highly active, e.g., the Cre-loxP or the FLPe system, and has enhanced thermostability (Rodriguez et al., 2000, Nature Genetics 25: 139-40; incorporated herein by reference in its entirety).

In certain embodiments, a recombinase system can be linked to a second inducible or repressible transcriptional regulation system. For example, a cell-specific Cre-loxP mediated recombination system (Gossen and Bujard, 1992, Proc. Natl. Acad. Sci. USA 89:

5547-51) can be linked to a cell-specific tetracycline-dependent time switch detailed above (Ewald et al., 1996, Science 273: 1384-1386; Furth et al. Proc. Natl. Acad. Sci. U.S.A. 91: 9302-06 (1994); St-Onge et al., 1996, Nucleic Acids Research 24(19): 3875-77; which are incorporated herein by reference in their entireties).

In one embodiment, an altered cre gene with enhanced expression in mammalian cells is used (Gorski and Jones, 1999, Nucleic Acids Research 27(9): 2059-61; incorporated herein by reference in its entirety).

In a specific embodiment, the ligand-regulated recombinase system of Kellendonk et al. (1999, J. Mol. Biol. 285: 175-82; incorporated herein by reference in its entirety) can be used. In this system, the ligand-binding domain (LBD) of a receptor, e.g., the progesterone or estrogen receptor, is fused to the Cre recombinase to increase specifity of the recombinase.

4.3. Vectors

In one aspect of the invention, the transgene is inserted into an appropriate vector. A vector is a nucleic acid molecule capable of transporting another nucleic acid to which it has been linked, preferably, the other nucleic acid is incorporated into the vector via a covalent linkage, more preferably via a nucleotide bond such that the other nucleic acid can be replicated along with the vector sequences. One type of vector is a plasmid, which is a circular double stranded DNA loop into which additional DNA segments can be ligated. Another type of vector is a viral vector, wherein additional DNA segments can be ligated into a viral genome or derivative thereof. Certain vectors are capable of autonomous replication in a host cell into which they are introduced (e.g., episomal mammalian vectors). Other vectors (e.g., non-episomal mammalian vectors) are integrated into the genome of a host cell upon introduction into the host cell, and thereby are replicated along with the host genome. The invention includes viral vectors, e.g., replication defective retroviruses, adenoviruses and adeno-associated viruses, which serve equivalent functions.

A large number of vector-host systems known in the art may be used. Possible vectors include, but are not limited to, plasmids or modified viruses, but the vector system must be compatible with the host cell used. Such vectors include, but are not limited to, bacteriophages such as lambda derivatives, or plasmids such as pBR322 or pUC plasmid derivatives or the Bluescript vector (Stratagene).

Preferably, vectors can replicate (i.e., have a bacterial origin of replication) and be manipulated in bacteria (or yeast) and can then be introduced into mammalian cells. Preferably, the vector comprises a selectable or detectable marker such as Ampr, tetr, LacZ, etc. The recombinant vectors of the invention comprise a transgene of the invention in a form suitable for expression of the nucleic acid in a transformed cell or transgenic animal. Preferably, such vectors can accommodate (i.e., can be used to introduce into cells and replicate) large pieces of DNA such as genomic sequences, for example, large pieces of DNA consisting of at least 25 kb, 50 kb, 75 kb, 100 kb, 150 kb, 200 kb or 250 kb, such as BACs, YACs, cosmids, etc. Preferably, the vector is a BAC.

The insertion of a DNA fragment into a vector can, for example, be accomplished by ligating the DNA fragment into a vector that has complementary cohesive termini. However, if the complementary restriction sites used to fragment the DNA are not present in the vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. In an alternative method, the cleaved vector and the transgene may be modified by homopolymeric tailing.

The vector can be cloned using methods known in the art, e.g.,by the methods disclosed in Sambrook et al., 2001, Molecular Cloning, A Laboratory Manual, Third Edition, Cold Spring Harbor Laboratory Press, N.Y.; Ausubel et al., 1989, Current Protocols in Molecular Biology, Green Publishing Associates and Wiley Interscience, N.Y., both of which are hereby incorporated by reference in their entireties. Vectors have replication origins and other selectable or detectable markers to allow selection of cells with vectors and vector maintenance. Preferably, the vectors contain cloning sites, for example, restriction enzyme sites that are unique in the sequence of the vector and insertion of a sequence at that site would not disrupt an essential vector function, such as replication.

In another aspect of the invention, a collection of vectors for making transgenic animals is provided. The collection comprises two or more vectors wherein each vectors comprises a transgene containing a system gene coding for a selectable or detectable marker protein operably linked to regulatory sequences of a characterizing gene corresponding to an endogenous gene or ortholog of an endogenous gene such that said system gene is expressed in said transgenic animal with an expression pattern that is substantially the same as the expression pattern of said endogenous gene in a non-transgenic animal or anatomical region or tissue thereof containing the population of cells of interest. The collection of vectors is used to make the collections of transgenic animal lines as described in Section 4. 1, supra.

4.3.1. Artificial Chromosomes

As discussed above, vectors used in the methods of the invention preferably can accommodate, and in certain embodiments comprise, large pieces of heterologous DNA such as genomic sequences. Such vectors can contain an entire genomic locus, or at least sufficient sequence to confer endogenous regulatory expression pattern and to insulate the expression of coding sequences from the effect of regulatory sequences surrounding the site of integration of the transgene in the genome to mimic better wild type expression. When entire genomic loci or significant portions thereof are used, few, if any, site-specific expression problems of a transgene are encountered, unlike insertions of transgenes into smaller sequences. In a preferred embodiment, the vector is a BAC containing genomic sequences into which system gene coding sequences have been inserted by directed homologous recombination in bacteria, e.g., the methods of Heintz WO 98/59060; Heintz et al., WO 01/05962; Yang et al., 1997, Nature Biotechnol. 15: 859-865; Yang et al., 1999, Nature Genetics 22: 327-35; which are incorporated herein by reference in their entireties.

Using such methods, a BAC can be modified directly in a recombination-deficient E. coli host strain by homologous recombination.

In a preferred embodiment, homologous recombination in bacteria is used for target-directed insertion of the system gene coding sequence into the genomic DNA encoding the characterizing gene and sufficient regulatory sequences to promote expression of the characterizing gene in its endogenous expression pattern, which sequences have been inserted into the BAC. The BAC comprising the system gene coding sequences under the regulation of the characterizing gene sequences is then recovered and introduced into the genome of a potential founder animal for a line of transgenic animals.

In specific embodiments, the system gene is inserted into the 3′ UTR of the characterizing gene and, preferably, has its own IRES. In another specific embodiment, the system gene is inserted into the characterizing gene sequences using 5′ direct fusion without the use of an IRES, i.e., such that the system gene coding sequences are fused directly in frame to the nucleotide sequence encoding at least the first codon of the characterizing gene coding sequence and even the first two, four, five, six, eight, ten or twelve codons. In yet another specific embodiment, the system gene is inserted into the 5′ UTR of the characterizing gene with an IRES controlling the expression of the system gene.

In a preferred aspect of the invention, the system gene sequence is introduced into the BAC containing the characterizing gene by the methods of Heintz el al. WO 98/59060 and Heintz et al., WO 01/05962, both of which are incorporated herein by reference in their entireties. The system gene is introduced by performing selective homologous recombination on a particular nucleotide sequence contained in a recombination deficient host cell, i.e.,a cell that cannot independently support homologous recombination, e.g., Rec A⁻. The method preferably employs a recombination cassette that contains a nucleic acid containing the system gene coding sequence that selectively integrates into a specific site in the characterizing gene by virtue of sequences homologous to the characterizing gene flanking the system gene coding sequences on the shuttle vector when the recombination deficient host cell is induced to support homologous recombination (for example by providing a functional RecA gene on the shuttle vector used to introduce the recombination cassette).

In a preferred aspect, the particular nucleotide sequence that has been selected to undergo homologous recombination is contained in an independent origin based cloning vector introduced into or contained within the host cell, and neither the independent origin based cloning vector alone, nor the independent origin based cloning vector in combination with the host cell, can independently support homologous recombination (e.g., is RecA⁻). Preferably, the independent origin based cloning vector is a BAC or a bacteriophage-derived artificial chromosome (BBPAC) and the host cell is a host bacterium, preferably E. coli. In another preferred aspect, sufficient characterizing gene sequences flank the system gene coding sequences to accomplish homologous recombination and target the insertion of the system gene coding sequences to a particular location in the characterizing gene. The system gene coding sequence and the homologous characterizing gene sequences are preferably present on a shuttle vector containing appropriate selectable markers and the RecA gene, optionally with a temperature sensitive origin of replication (see Heintz et al. WO 98/59060 and Heintz et al., WO01/05962 such that the shuttle vector only replicates at the permissive temperature and can be diluted out of the host cell population at the non-permissive temperature. When the shuttle vector is introduced into the host cell containing the BAC the RecA gene is expressed and recombination of the homologous shuttle vector and BAC sequences can occur thus targeting the system gene coding sequences (along with the shuttle vector sequences and flanking characterizing gene sequences) to the characterizing gene sequences in the BAC. The BACs can be selected and screened for integration of the system gene coding sequences into the selected site in the characterizing gene sequences using methods well known in the art (e.g., methods described in Section 5, infra, and in Heintz et al. WO 98/59060 and Heintz et al., WO01/05962). Optionally, the shuttle vector sequences not containing the system gene coding sequences (including the RecA gene and any selectable markers) can be removed from the BAC by resolution as described in Section 5 and in Heintz et al. WO 98/59060 and Heintz et al., WO 01/05962. If the shuttle vector contains a negative selectable marker, cells can be selected for loss of the shuttle vector sequences. In an alternative embodiment, the functional RecA gene is provided on a second vector and removed after recombination, e.g., by dilution of the vector or by any method known in the art. The exact method used to introduce the system gene coding sequences and to remove (or not) the RecA (or other appropriate recombination enzyme) will depend upon the nature of the BAC library used (for example the selectable markers present on the BAC vectors) and such modifications are within the skill in the art. Once the BAC containing the characterizing gene regulatory sequences and system gene coding sequences in the desired configuration is identified, it can be isolated from the host E. coli cells using routine methods and used to make transgenic animals as described in Sections 4.4 and 4.5, infta.

BACs to be used in the methods of the invention are selected and/or screened using the methods described in Section 4.2, supra, and Section 5, infra.

Alternatively, the BAC can also be engineered or modified by “E-T cloning,” as described by Muyrers et al. (1999, Nucleic Acids Res. 27(6): 1555-57, incorporated herein by reference in its entirety). Using these methods, specific DNA may be engineered into a BAC independently of the presence of suitable restriction sites. This method is based on homologous recombination mediated by the recE and recI proteins (“ET-cloning”) (Zhang et al., 1998, Nat. Genet. 20(2): 123-28; incorporated herein by reference in its entirety). Homologous recombination can be performed between a PCR fragment flanked by short homology arms and an endogenous intact recipient such as a BAC. Using this method, homologous recombination is not limited by the disposition of restriction endonuclease cleavagte sites or the size of the target DNA. A BAC can be modified in its host strain using a plasmid, e.g., pBAD-αβγ, in which recE and recT have been replaced by their respective functional counterparts of phage lambda (Muyrers et al., 1999, Nucleic Acids Res. 27(6): 1555-57). Preferably, a BAC is modified by recombination with a PCR product containing homology arms ranging from 27-60 bp. In a specific embodiment, homology arms are 50 bp in length.

In another embodiment, a transgene is inserted into a yeast artificial chromosome (YAC) (Burke et al., 1987 Science 236: 806-12; and Peterson et al., 1997, Trends Genet. 13: 61).

In other embodiments, the transgene is inserted into another vector developed for the cloning of large segments of mammalian DNA, such as a cosmid or bacteriophage P1 (Sternberg et al., 1990, Proc. Natl. Acad. Sci. USA 87: 103-07). The approximate maximum insert size is 30-35 kb for cosmids and 100 kb for bacteriophage P1.

In another embodiment, the transgene is inserted into a P-1 derived artificial chromosome (PAC) (Mejia et al., 1997, Genome Res 7:179-186). The maximum insert size is 300 kb.

Vectors containing the appropriate characterizing and system gene sequences may be identified by any method well known in the art, for example, by sequencing, restriction mapping, hybridization, PCR amplification, etc.

Retroviruses may also be used as vectors for introducing genetic material into mammalian genomes. They provide high efficiency infection, stable integration and stable expression (Friedmann, 1989, Science 244: 1275-81). Genomic sequences of a gene of interest, e.g., a system gene and/or a characterizing gene, or portions thereof can be cloned into a retroviral vector. Delivery of the virus can be accomplished by direct injection or implantation of virus into the desired tissue of the adult animal, a fertilized egg, early stage or later stage embryos.

In one embodiment, a promoter or other regulatory sequence of a characterizing gene and a system gene cDNA are cloned into a retrovirus vector.

Transient transfection can be used to assess transgene activity. Stable intracellular expression of an active transgene can be achieved by viral vector-mediated delivery. Retroviral vectors are preferable because they permit stable integration of the transgene into a dividing host cell genome, and the absence of any viral gene expression reduces the chance of an immune response in the transgenic animal. In addition, retroviruses can be easily pseudo-typed with a variety of envelope proteins to broaden or restrict host cell tropism, thus adding an additional level of cellular targeting for transgene delivery (Welch et al., 1998, Curr. Opin. Biotechnol. 9: 486-96).

Adenoviral vectors can be used to provide efficient transduction, but they do not integrate into the host genome and, consequently, expression of the transgenes is only transient in actively dividing cells. In animals, a further complication arises in that the most commonly used recombinant adenoviral vectors still contain viral late genes that are expressed at low levels and can lead to a host immune response against the transduced cells (Welch et al., 1998, Curr. Opin. Biotechnol. 9: 486-96). In one embodiment, a ‘gutless’ adenoviral vector can be used that lacks all viral coding sequences (Parks et al., 1996, Proc. Natl. Acad. Sci. USA 93: 13565-70; incorporated herein by reference in its entirety).

Other delivery systems which can be utilized include adeno-associated virus (AAV), lentivirus, alpha virus, vaccinia virus, bovine papilloma virus, members of the herpes virus group such as Epstein-Barr virus, baculovirus, yeast vectors, bacteriophage vectors (e.g., lambda), and plasmid and cosmid DNA vectors. Viruses with tropism to central nervous system (CNS) tissue are also envisioned.

Adeno-associated virus is attractive as a small, non-pathogenic virus that can stably integrate a transgene expression cassette without any viral gene expression (Welch et al., 1998, Curr. Opin. Biotechnol. 9: 486-96). An alpha virus system, using recombinant Semliki Forest virus, provides high transduction efficiencies of mammalian cells along with high cytoplasmic transgene, e.g., ribozyme, expression (Welch et al., 1998, Curr. Opin. Biotechnol. 9: 486-96). Finally, lentiviruses (such as HIV and feline immunodeficiency virus) are attractive as gene delivery vehicles due to their ability to integrate into non-dividing cells (Welch et al., 1998, Curr. Opin. Biotechnol. 9: 486-96).

Site-specific integration of a transgene can be mediated by an adeno-associated virus (AAV) vector derived from a nonpathogenic and defective human parvovirus. In one embodiment, a recombinant adeno-associated virus (rAAV) is used to mediate transgene integration in a population of nondividing cells (Wu et al., 1998, J. Virol. 72(7): 5919-26; incorporated herein by reference in its entirety). In a specific embodiment, the nondividing cells are neurons.

In another embodiment, a recombinant (non-wildtype) AAV (rAAV) is used, such as one of those disclosed by Xiao et al. (1997, Exper. Neurol. 144: 113-24; incorporated herein by reference in its entirety). Such an rAAV vector has biosafety features, a high titer, broad host range, lacks cytotoxicity, does not evoke a cellular immune response in the target tissue, and transduces quiescent or non-dividing cells. It is preferably used to transduce ceils in the central nervous system (CNS). In another embodiment, rAAV plasmid DNA is used in a nonviral gene delivery system as disclosed by Xiao et al. (1997, Exper. Neurol. 144: 113-24).

A replication-defective lentiviral vector, such as the one described by Naldini et al. (1996, Proc. Natl. Acad. Sci. USA 93: 11382-88: incorporated herein by reference in its entirety), can be used for in vivo delivery of a transgene. Preferably, the reverse transcription of the vector is promoted inside the vector particles before delivery to enhance the efficiency of gene transfer. The lentiviral vector may be injected into a specific tissue, e.g., the brain.

In another embodiment, a lentivirus-based vector capable of infecting both mitotic and postmitotic cells is used for targeted gene transfer. Postmitotic cells, in particular postmitotic neurons, are generally refractory to stable infection by retroviral vectors, which require the breakdown of the nuclear membrane during cell division in order to insert the transgene into the host cell genome. Therefore, in a preferred embodiment, a lentivirus vector based on the human immunodeficiency virus (HIV) (Blömer et al., 1997, J. Virol., Vol. 71(9): 6641-49; incorporated herein by reference in its entirety) is used to infect and stably transduce dividing as well as terminally differentiated cells, preferably neurons. (for a review of lentivirus vectors suitable for infecting non-dividing cells, see Naldini, 1998, Curr. Opin. Biotechnol. 9: 457-63).

Nondividing cells can be infected by human immunodeficiency virus type 1 (HIV-1)-based vectors, which results in transgene expression that is stable over several months. Preferably, an HIV-1 vector with biosafety features, e.g., a self-inactivating HIV-1 vector is used. In one embodiment, a self-inactivating HIV-1 vector with a 400-nucleotide deletion in the 3′ long terminal repeat (LTR) is used (Zufferey et al., 1998, J. Virol. 72(12): 9873-80; incorporated herein by reference in its entirety). The deletion, which includes the TATA box, abolishes the LTR promoter activity but does not affect vector titers or transgene expression in vitro. The self-inactivating vector may be used to transduce neurons in vivo.

In another embodiment, a retroviral vector that is rendered replication incompetent, stably integrates into the host cell genome, and does not express any viral proteins, such as a vector based on the Moloney murine leukemia virus (MMLV), is used for gene transfer into the host cell genome (Blomer et al., 1997, J. Virol., Vol. 71(9): 6641-49).

4.4. Introduction of Vectors Into Host Cells

In one aspect of the invention, a vector containing the transgene comprising the system and/or characterizing gene is introduced into the genome of a host cell, and the host cell is then used to create a transgenic animal. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells), preferably a mammalian cell, and most preferably a mouse cell. Host cells intended to be part of the invention include ones that comprise a system and/or characterizing gene sequence that has been engineered to be present within the host cell (e.g., as part of a vector), and ones that comprise nucleic acid regulatory sequences that have been engineered to be present in the host cell such that a nucleic acid molecule of the invention is expressed within the host cell. The invention encompasses genetically engineered host cells that contain any of the foregoing system and/or characterizing gene sequences operatively associated with a regulatory element (preferably from a characterizing gene, as described above) that directs the expression of the coding sequences in the host cell. Both cDNA and genomic sequences can be cloned and expressed. In a preferred aspect, the host cell is recombination deficient, i.e., Rec⁻, and used for BAC recombination.

A vector containing a transgene can be introduced into the desired host cell by methods known in the art, e.g., transfection, transformation, transduction, electroporation, infection, microinjection, cell fusion, DEAE dextran, calcium phosphate precipitation, liposomes, LIPOFECTIN™ (source), lysosome fusion, synthetic cationic lipids, use of a gene gun or a DNA vector transporter, such that the transgene is transmitted to offspring in the line. For various techniques for transformation or transfection of mammalian cells, see Keown et al., 1990, Methods Enzymol. 185: 527-37; Sambrook et al., 2001, Molecular Cloning, A Laboratory Manual, Third Edition, Cold Spring Harbor Laboratory Press, N.Y.

Particularly preferred embodiments of the invention encompass methods of introduction of the vector containing the transgene using pronuclear injection of a transgenic construct into the mononucleus of a mouse embryo and infection with a viral vector comprising the construct. Methods of pronuclear injection into mouse embryos are well-known in the art and described in Hogan et al. 1986, Manipulating the Mouse Embryo, Cold Spring Harbor Laboratory Press, New York, N.Y. and Wagner ei al., U.S. Pat. No. 4,873,191, issued Oct. 10, 1989, herein incorporate by reference in their entireties.

In preferred embodiments, a vector containing the transgene is introduced into any nucleic genetic material which ultimately forms a part of the nucleus of the zygote of the animal to be made transgenic. including the zygote nucleus. In one embodiment, the transgene can be introduced in the nucleus of a primordial germ cell which is diploid, e.g., a spermatogonium or oogonium. The primordial germ cell is then allowed to mature to a gamete which is then united with another gamete or source of a haploid set of chromosomes to form a zygote. In another embodiment, the vector containing the transgene is introduced in the nucleus of one of the gametes, e.g., a mature sperm, egg or polar body, which forms a part of the zygote. In preferred embodiments, the vector containing the transgene is introduced in either the male or female pronucleus of the zygote. More preferably, it is introduced in either the male or the female pronucleus as soon as possible after the sperm enters the egg. In other words, right after the formation of the male pronucleus when the pronuclei are clearly defined and are well separated, each being located near the zygote membrane.

In a most preferred embodiment, the vector containing the transgene is added to the male DNA complement, or a DNA complement other than the DNA complement of the female pronucleus, of the zygote prior to its being processed by the ovum nucleus or the zygote female pronucleus. In an alternate embodiment, the vector containing the transgene could be added to the nucleus of the sperm after it has been induced to undergo decondensation. Additionally, the vector containing the transgene may be mixed with sperm and then the mixture injected into the cytoplasm of an unfertilized egg. Perry et al., 1999, Science 284:1180-1183. Alternatively, the vector may be injected into the vas deferens of a male mouse and the male mouse mated with normal estrus females. Huguet et al., 2000, Mol. Reprod. Dev. 56:243-247.

Preferably, the transgene is introduced using any technique so long as it is not destructive to the cell,nuclear membrane or other existing cellular or genetic structures. The transgene is preferentially inserted into the nucleic genetic material by microinjection. Microinjection of cells and cellular structures is known and is used in the art. Also known in the art are methods of transplanting the embryo or zygote into a pseudopregnant female where the embryo is developed to term and the transgene is integrated and expressed. See, e.g., Hogan et al. 1986, Manipulating the Mouse Embryo, Cold Spring Harbor Laboratory Press, New York, N.Y.

Viral methods of inserting a transgene are known in the art and have been described, supra.

For stable transfection of cultured mammalian cells, only a small fraction of cells may integrate the foreign DNA into their genome. The efficiency of integration depends upon the vector and transfection technique used. In order to identify and select integrants, a gene that encodes a selectable marker (e.g., for resistance to antibiotics) is generally introduced into the host cells along with the gene sequence of interest e.g., the system gene sequence. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die). Such methods are particularly useful in methods involving homologous recombination in mammalian cells (e.g., in murine ES cells) prior to introducing the recombinant cells into mouse embryos to generate chimeras.

A number of selection systems may be used to select transformed host cells. In particular, the vector may contain certain detectable or selectable markers. Other methods of selection include but are not limited to selecting for another marker such as: the herpes simplex virus thymidine kinase (Wigler et al., 1977, Cell 11: 223), hypoxanthine-guanine phosphoribosyltransferase (Szybalska and Szybalski, 1962, Proc. Natl. Acad. Sci. USA 48: 2026), and adenine phosphoribosyltransferase (Lowy et al., 1980, Cell 22: 817) genes can be employed in tk−, hgprt− or aprt− cells, respectively. Also, antimetabolite resistance can be used as the basis of selection for the following genes: dhfr, which confers resistance to methotrexate (Wigler et al., 1980, Natl. Acad. Sci. USA 77: 3567; O'Hare et al., 1981, Proc. Natl. Acad. Sci. USA 78: 1527); gpt, which confers resistance to mycophenolic acid (Mulligan and Berg, 1981, Proc. Natl. Acad. Sci. USA 78: 2072); neo, which confers resistance to the aminoglycoside G-418 (Colberre-Garapin et al., 1981, J. Mol. Biol. 150: 1); and hygro, which confers resistance to hygromycin (Santerre et al., 1984, Gene 30: 147).

The transgene may integrate into the genome of the founder animal (or an oocyte or embryo that gives rise to the founder animal), preferably by random integration. In other embodiments the transgene may integrate by a directed method, e.g., by directed homologous recombination (“knock-in”), Chappel, U.S. Pat. No. 5,272,071; and PCT publication No. WO 91/06667, published May 16, 1991; U.S. Pat. No. 5,464,764; Capecchi et al., issued Nov. 7, 1995; U.S. Pat. No. 5,627,059, Capecchi et al. issued, May 6, 1997; U.S. Pat. No. 5,487,992, Capecchi et al., issued Jan. 30, 1996). Preferably, when homologous recombination is used, it does not knock out or replace the host's endogenous copy of the characterizing gene (or characterizing gene ortholog).

Methods for generating cells having targeted gene modifications through homologous recombination are known in the art. The construct will comprise at least a portion of the characterizing gene with a desired genetic modification, e.g., insertion of the system gene coding sequences and will include regions of homology to the target locus, i.e., the endogenous copy of the characterizing gene in the host's genome. DNA constructs for random integration need not include regions of homology to mediate recombination. Markers can be included for performing positive and negative selection for insertion of the transgene.

To create a homologous recombinant animal, a homologous recombination vector is prepared in which the system gene is flanked at its 5′ and 3′ ends by characterizing gene sequences to allow for homologous recombination to occur between the exogenous gene carried by the vector and the endogenous characterizing gene in an embryonic stem cell. The additional flanking nucleic acid sequences are of sufficient length for successful homologous recombination with the endogenous characterizing gene. Typically, several kilobases of flanking DNA (both at the 5′ and 3′ ends) are included in the vector. Methods for constructing homologous recombination vectors and homologous recombinant animals are described further in Thomas and Capecchi, 1987, Cell 51: 503; Bradley, 1991, Curr. Opin. Bio/Technol. 2: 823-29; and PCT Publication Nos. WO 90/11354, WO 91/01140, WO 92/0968, and WO 93/04169.

4.5. Methods of Producing Transgenic Animals

A transgenic animal is a non-human animal, preferably a mammal, more preferably a rodent such as a rat or mouse, in which one or more of the cells of the animal includes a transgene, i.e., has a non-endogenous (i.e., heterologous) nucleic acid sequence present as an extrachromosomal element in a portion of its cell or stably integrated into its germ line DNA (i.e., in the genomic sequence of most or all of its cells). Other examples of transgenic animals include non-human primates, sheep, dogs, cows, goats, chickens, amphibians, etc. Unless otherwise indicated, it will be assumed that a transgenic animal comprises stable changes to the germline sequence. Heterologous nucleic acid is introduced into the germ line of such a transgenic animal by genetic manipulation of, for example, embryos or embryonic stem cells of the host animal.

As discussed above, the transgenic animals of the invention are preferably generated by random integration of a vector containing a transgene of the invention into the genome of the animal, for example, by pronuclear injection in the animal zygote, or injection of sperm mixed with vector DNA as described above. Other methods involve introducing the vector into cultured embryonic cells, for example ES cells, and then introducing the transformed cells into animal blastocysts, thereby generating a “chimeras” or “chimeric animals”, in which only a subset of cells have the altered genome. Chimeras are primarily used for breeding purposes in order to generate the desired transgenic animal. Animals having a beterozygous alteration are generated by breeding of chimeras. Male and female heterozygotes are typically bred to generate homozygous animals.

A homologous recombinant animal is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

In a, preferred embodiment, a transgenic animal of the invention is created by introducing a transgene of the invention, encoding the characterizing gene regulatory sequences operably linked to the system gene sequence, into the male pronuclei of a fertilized oocyte, e.g., by microinjection or retroviral infection, and allowing the egg to develop in a pseudopregnant female foster animal. Methods for generating transgenic animals via embryo manipulation and microinjection, particularly animals such as mice, have become conventional in the art and are described, for example, in U.S. Pat. Nos. 4,736,866 and 4,870,009, 4,873,191, in Hogan, Manipulating the Mouse Embryo, (Cold Spring Harbor Laboratory Press, Cold Spring Harbor, N.Y., 1986) and in Wakayama et al., 1999, Proc. Natl. Acad. Sci. USA, 96:14984-89; see also infta. Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene as described supra. Moreover, transgenic animals carrying the transgene can further be bred to other transgenic animals carrying other transgenes, animals of the same species that are disease models, etc.

In another embodiment, the transgene is inserted into the genome of an embryonic stem (ES) cell, followed by injection of the modified ES cell into a blastocyst-stage embryo that subsequently develops to maturity and serves as the founder animal for a line of transgenic animals.

In another embodiment, a vector bearing a transgene is introduced into ES cells (e.g., by electroporation) and cells in which the introduced gene has homologously recombined with the endogenous gene are selected. See, e.g., Li et al., 1992, Cell 69:915. For embryonic stem (ES) cells, an ES cell line may be employed, or embryonic cells may be obtained freshly from a host, e.g. mouse, rat, guinea pig, etc.

After transformation, ES cells are grown on an appropriate feeder layer, e.g., a fibroblast-feeder layer, in an appropriate medium and in the presence of appropriate growth factors, such as leukemia inhibiting factory (LIF). Cells that contain the construct may be detected by employing a selective medium. Transformed ES cells may then be used to produce transgenic animals via embryo manipulation and blastocyst injection. (See, e.g. U.S. Pat. Nos. 5,387,742, 4,736,866 and 5,565,186 for methods of making transgenic animals.)

Stable expression of the construct is preferred. For example, ES cells that stably express a system gene product may be engineered. Rather than using vectors that contain viral origins of replication, ES host cells can be transformed with DNA, e.g., a plasmid, controlled by appropriate expression control elements (e.g., promoter, enhancer, sequences, transcription terminators, polyadenylation sites, etc.), and a selectable marker. Following the introduction of the foreign DNA, engineered ES cells may be allowed to grow for 1-2 days in an enriched media, and then are switched to a selective media. The selectable marker in the recombinant plasmid confers resistance to the selection and allows cells to stably integrate the plasmid into their chromosomes and expanded into cell lines. This method may advantageously be used to engineer ES cell lines that express the system gene product.

The selected ES cells are then injected into a blastocyst of an animal (e.g., a mouse) to form aggregation chimeras. See, e.g., Bradley, 1987, in Teratocarcinomas and Embryonic Stem Cells: A Practical Approach, Robertson, ed., IRL, Oxford, 113-52. Blastocysts are obtained from 4 to 6 week old superovulated females. The ES cells are trypsinized, and the modified cells are injected into the blastocoel of the blastocyst. After injection, the blastocysts are implanted into the uterine horns of suitable pseudopregnant female foster animal. Alternatively, the ES cells may be incorporated into a morula to form a morula aggregate which is then implanted into a suitable pseudopregnant femal foster animal. Females are then allowed to go to term and the resulting litters screened for mutant cells having the construct.

The chimeric animals are screened for the presence of the modified gene. By providing for a different phenotype of the blastocyst and the ES cells, chimeric progeny can be readily detected. Males and female chimeras having the modification are mated to produce homozygous progeny. Only chimeras with transformed germline cells will generate homozygous progeny. If the gene alterations cause lethality at some point in development, tissues or organs can be maintained as allergenic or congenic grafts or transplants, or in in vitro culture.

Progeny harboring homologously recombined or integrated DNA in their germline cells can be used to breed animals in which all cells of the animal contain the homologously recombined DNA or randomly integrated transgene by germline transmission of the transgene.

Clones of the non-human transgenic animals described herein can also be produced according to the methods described in Wilmut et al., 1997, Nature 385: 810-13 and PCT Publication NOS. WO 97/07668 and WO 97/07669.

Once the transgenic mice are generated they may be bred and maintained using methods well known in the art. By way of example, the mice may be housed in an environmentally controlled facility maintained on a 10 hour dark: 14 hour light cycle or other appropriate light cycle. Mice are mated when they are sexually mature (6 to 8 weeks old). In certain embodiments, the transgenic founders or chimeras are mated to an unmodified animal (i.e., an animal having no cells containing the transgene). In a preferred embodiment, the transgenic founder or chimera is mated to C57BL/6 mice (Jackson Laboratories). In a specific embodiment where the transgene is introduced into ES cells and a chimeric mouse is generated, the chimera is mated to 129/Sv mice, which have the same genotype as the embryonic stem cells. Protocols for successful breeding are known in the art (See hhtp://www.informaticsjax.org/mgihome). Preferably, a founder male is mated with two females and a founder female is mated with one male. Preferably two females are rotated through a male's cage every 1-2 weeks. Pregnant females are generally housed 1 or 2 per cage. Preferably, pups are ear tagged, genotyped, and weaned at approximately 21 days. Males and females are housed separately. Preferably log sheets are kept for any mated animal, by example and not limitation, information should include pedigree, birth date, sex, ear tag number, source of mother and father, genotype, dates mated and generation.

More specifically, founder animals heterozygous for the transgene may be mated to generate a homozygous line as follows: A heterzygous founder animal, designated as the P₁ generation, is mated with an offspring designated as the F₁ generation from a mating of a non-transgenic mouse with a transgenic mouse heterozygous for the transgene (backcross). Based on classical genetics, one fourth of the results of this backcross are homozygous for the transgene. In a preferred embodiment, transgenic founders are individually backcrossed to an inbred or outbred strain of choice. Different founders should not be intercrossed, since different expression patterns may result from separate transgene integration events.

The determination of whether a transgenic mouse is homozygous or heterozygous for the transgene is as follows:

An offspring of the above described breeding cross is mated to a normal control non-transgenic animal. The offspring of this second mating are analyzed for the presence of the transgene by the methods described below. If all offspring of this cross test positive for the transgene, the mouse in question is homozygous for the transgene. If, on the other hand, some of the offspring test positive for the transgene and others test negative, the mouse in question is heterozygous for the transgene.

An alternative method for distinguishing between a transgenic animal which is heterozygous and one which is homozygous for the transgene is to measure the intensity with radioactive probes following Southern blot analysis of the DNA of the animal. Animals homozygous for the transgene would be expected to produce higher intensity signals from probes specific for the transgene than would heterozygote transgenic animals.

In a preferred embodiment, the transgenic mice are so highly inbred to be genetically identical except for sexual differences. The homozygotes are tested using backcross and intercross analysis to ensure homozygosity. Homozygous lines for each integration site in founders with multiple integrations are also established. Brother/sister matings for 20 or more generations define an inbred strain. In another preferred embodiment, the transgenic lines are maintained as hemizygotes.

In an alternative embodiment, individual genetically altered mouse strains are also cryopreserved rather than propagated. Methods for freezing embryos for maintenance of founder animals and transgenic lines are known in the art. Gestational day 2.5 embryos are isolated and cryopreserved in straws and stored in liquid nitrogen. The first and last straw are subsequently thawed and transferred to foster females to demonstrate viability of the line with the assumption that all embryos frozen between the first and last straw will behave similarly. If viable progeny are not observed a second embryo transfer will be performed. Methods for reconstituting frozen embryos and bringing the embryos to term are known in the art.

4.6. Methods of Screening for Expression of Transgenes

In preferred embodiments, the invention provides a collection of such transgenic animal lines comprising at least two individual lines, preferably at least five individual lines. Each individual line is selected for the collection based on the identity of the subset of cells in which the system gene is expressed.

Potential founder animals for a line of transgenic animals can be screened for expression of the system gene sequence in the population of cells characterized by expression of the endogenous characterizing gene.

Transgenic animals that exhibit appropriate expression (e.g., detectable expression having substantially the same expression pattern as the endogenous characterizing gene in a corresponding non-transgenic animal or anatomical region thereof, i.e., detectable expression in at least 80%, 90%, 95% or, preferably 100% of the cells shown to express the endogenous gene by in situ hybridization) are selected as transgenic animal lines.

Additionally, in situ hybridization using probes specific for the system gene coding sequences may also be used to detect expression of the system gene product.

In a preferred embodiment, immunohistochemistry using an antibody specific for the system gene product or marker activated or repressed thereby is used to detect expression of the system gene product.

In another aspect of the invention, system gene expression is visualized in single living mammalian cells. In one embodiment, the method of Zlokarnik et al., (1998, Science 279: 84-88; incorporated herein by reference in its entirety) is used to visualize system gene expression. The system gene encodes an enzyme, e.g., β-lactamase. To image single living cells, an enzyme assay is performed in which β-lactamase hydrolyzes a substrate loaded intracellularly as a membrane-permeant ester. Each molecule of β-lactamase changes the fluorescence of many substrate molecules from green to blue by disrupting resonance energy transfer. This wavelength shift can be detected by eye or photographically (either on film or digitally) in individual cells containing less than 100 β-lactamase molecules.

In another embodiment, the non-invasive method of Contag et al. is used to detect and localize light originating from a mammal in vivo (Contag et al., U.S. Pat. No. 5,650,135, issued Jul. 22, 1997; incorporated herein by reference in its entirety) . Light-emitting conjugates are used that contain a biocompatible entity and a light-generating moiety. Biocompatible entities include, but are not limited to, small molecules such as cyclic organic molecules; macromolecules such as proteins; microorganisms such as viruses, bacteria, yeast and fungi; eukaryotic cells; all types of pathogens and pathogenic substances; and particles such as beads and liposomes. In another aspect, biocompatible entities may be all or some of the cells that constitute the mammalian subject being imaged.

Light-emitting capability is conferred on the entities by the conjugation of a light-generating moiety. Such moieties include fluorescent molecules, fluorescent proteins, enzymatic reactions giving off photons and luminescent substances, such as bioluminescent proteins. The conjugation may involve a chemical coupling step, genetic engineering of a fusion protein, or the transformation of a cell, microorganism or animal to express a bioluminescent protein. For example, in the case where the entities are the cells constituting the mammalian subject being imaged, the light-generating moiety may be a bioluminescent or fluorescent protein “conjugated” to the cells through localized, promoter-controlled expression from a vector construct introduced into the cells by having made a transgenic or chimeric animal.

Light-emitting conjugates are typically administered to a subject by any of a variety of methods, allowed to localize within the subject, and imaged. Since the imaging, or measuring photon emission from the subject, may last up to tens of minutes, the subject is usually, but not always, immobilized during the imaging process.

Imaging of the light-emitting entities involves the use of a photodetector capable of detecting extremely low levels of light—typically single photon events—and integrating photon emission until an image can be constructed. Examples of such sensitive photodetectors include devices that intensify the single photon events before the events are detected by a camera, and cameras (cooled, for example, with liquid nitrogen) that are capable of detecting single photons over the background noise inherent in a detection system.

Once a photon emission image is generated, it is typically superimposed on a “normal” reflected light image of the subject to provide a frame of reference for the source of the emitted photons (i.e. localize the light-emitting conjugates with respect to the subject). Such a “composite” image is then analyzed to determine the location and/or amount of a target in the subject.

4.7. Isolation and Purification of Cells from the Transgenic Animals

Homogeneous populations of cells can be isolated and purified from transgenic animals of the collection. Methods for cell isolation include, but are not limited to, surgical excision or dissection, dissociation, fluorescence-activated cell sorting (FACS), panning, and laser capture microdissection (LCM).

In certain embodiments, cells are isolated using surgical excision or dissection. Before dissection, the transgenic animal may be perfused. Perfusion is preferably accomplished using a perfusion solution that contains a-amanitin or other transcriptional blockers to prevent changes in gene expression from occurring during cell isolation.

In other embodiments, cells are isolated from adult rodent brain tissue which is dissected and dissociated. Methods for such dissection and dissociation are well-known in the art. See, e.g., Brewer, 1997, J. Neurosci. Methods 71(2):143-55; Nakajima et al., 1996, Neurosci. Res. 26(2):195-203; Masuko et al., 1992, Neuroscience 49(2):347-64; Baranes et al., 1996, Proc. Natl. Acad. Sci. USA 93(10):4706- 11; Emerling et al., 1994, Development 120(10):2811-22; Martinou (1989, J. Neurosci. 9(10):3645-56; Ninomiya, 1994, Int. J. Dev. Neurosci. 12(2): 99-106; Delree, 1989, J. Neurosci. Res. 23(2):198-206; Gilabert, 1997, J. Neurosci. Methods 71(2):191-98; Huber, 2000, J. Neurosci. Res. 59(3):372-78; which ar incorporated herein by reference in their entireties.

In other embodiments cells are dissected from tissue slices based on their morphology as seen by transmittance light direct visualization and cultured, using, e.g., the methods of Nakajima et al., 1996, Neurosci. Res. 26(2):195-203; Masuko et al., 1992, Neuroscience 49(2):347-64; which are incorporated herein by reference in their entireties. Tissue slices are made of a particular tissue region and a particular subregion, e.g., a brain nucleus, is isolated under direct visualization using a dissecting microscope.

In yet other embodiments, cells can be dissociated using a protease such as papain (Brewer, 1997, J. Neurosci. Methods 71(2):143-55; Nakajima et al., 1996, Neurosci. Res. 26(2):195-203;) ortrypsin (Baranes, 1996, Proc. Natl. Acad. Sci. USA 93(10):4706-11; Emerling et al., 1994, Development 120(10):2811-22; Gilabert, 1997, J. Neurosci. Methods 71(2):191-98; Ninomiya, 1994, Int. J. Dev. Neurosci. 12(2): 99-106; Huber, 2000, L. Neurosci. Res. 59(3):372-78; which are incorporated herein by reference in their entireties). Cells can also be dissociated using collagenase (Delree, 1989, J. Neurosci. Res. 23(2):198-206; incorporated herein by reference in its entirety). The dissociated cells are then grown in cultures over a feeder layer. In one embodiment, the dissociated cells are neurons that are grown over a glial feeder layer.

In another embodiment, tissue that is labeled with a fluorescent marker, e.g., a system gene protein, can be microdissected and dissociated using the methods of Martinou (1989, J. Neurosci. 9(10):3645-56). Microdissection of the labeled cells is followed by density-gradient centrifugation. The cells are then purified by fluorescence-activated cell sorting (FACS). In other embodiments, cells can be purified by a cell-sorting procedure that only uses light-scatter parameters and does not necessitate labeling (Martinou, 1989, J. Neurosci. 9(10):3645-56, incorporated herein by reference in its entirety).

In one aspect of the invention, a subset of cells within a heterogeneous cell population derived from a transgenic animal in the collection of transgenic animals lines is recognized by expression of a system gene. The regulatory sequences of the characterizing gene are used to express a system gene encoding a marker protein in transgenic cells, and the targeted population of cells is isolated based on expression of the system gene marker. Selection and/or separation of the target subpopulation of cells may be effected by any convenient method. For example, where the marker is an externally accessible, cell-surface associated protein or other epitope-containing molecule, immuno-adsorption panning techniques or fluorescent immuno-labeling coupled with fluorescence activated cell sorting (FACS) are conveniently applied.

Cells that express a system gene product, e.g., an enzyme can be detected using flow cytometric methods such as the one described by Mouawad et al., 1997, J. Immunol. Methods, 204(1), 51-56; incorporated herein by reference in its entirety). The method is based on an indirect immunofluorescence staining procedure using a monoclonal antibody that binds specifically to the marker enzyme encoded by the system gene sequence, e.g., β-galactosidase or a β-galactosidase fusion protein. The method can be used for both quantification in vitro and in vivo of enzyme expression in mammalian cells. The method is preferably used with a construct containing a lacZ selectable marker. Using such a method, cells expressing a system gene can be quantified and gene regulation, including transfection modality, promoter efficacy, enhancer activity, and other regulatory factors studied (Mouawad et al., 1997, J. Immunol. Methods 204(1): 51-56).

In another embodiment, a FACS-enzyme assay, e.g., a FACS-Gal assay, is used (see, e.g., Fiering et al., 1991, Cytometry 12(4): 291-301; Nolan et al., 1988, Proc. Natl. Acad. Sci. USA 85(8): 2603-07; which are incorporated herein by reference in their entireties). The FACS-Gal assay measures E. coli lacZ-encoded β-galactosidase activity in individual cells. Enzyme activity is measured by flow cytometry, using a fluorogenic substrate that is hydrolyzed and retained intracellularly. In the system described by Fiering et al., lacZ serves both as a reporter gene to quantitate gene expression and as a selectable marker for the fluorescence-activated cell sorting based on their lacZ expression level. Preferably, phenylethyl-beta-D-thiogalactoside (PETG), is used as a competitive inhibitor in the reaction, to inhibit β-galactosidase activity and slow reaction with the substrate. Also preferably, interfering endogenous host (e.g., mammalian) β-galactosidases are inhibited by the weak base chloroquine. Further, false positives may be minimized by performing two-color measurements (false-positive cells tend to fluoresce more in the yellow wavelengths.

In another specific embodiment, a fluorescence-activated cell sorter (FACS) is used to detect the activity of a system gene encoding E. coli β-glucuronidase (gus) (Lorincz et al., 1996, Cytometry 24(4): 321-9). When loaded with the Gus substrate fluorescein-di-beta-D-glucuronide (FDGlcu), individual mammalian cells expressing and translating gus mRNA liberate sufficient levels of intracellular fluorescein for quantitative analysis by flow cytometry. This assay can be used to FACS-sort viable cells based on Gus enzymatic activity, and the efficacy of the assay can be measured independently by using a fluorometric lysate assay. In another specific embodiment, the intracellular fluorescence generated by the activity of both beta-glucuronidase and E. coli β-galactosidase enzymes are detected by FACS independently. Because each enzyme has high specificity for its cognate substrate, each reporter gene can be measured by FACS independently.

The invention provides methods for isolating individual cells harboring a fluorescent protein reporter from tissues of transgenic mice by FACS. See Hadjaantonakis and Naki, 2000, Genesis, 27(3):95-8, which is incorporated herein by reference it its entirety. In certain embodiments of the invention, the reporter is a autofluorescent (AFP) reporter such as but not limited to wild type Green Fluorescent Protein (wtGFP) and its variants, including enhanced green fluorescent protein (EGFP) and enhanced yellow fluorescent protein (EYFP).

In one embodiment of the invention, cells are isolated by FACS using fluorescent antibody staining of cell surface proteins. The cells are isolated using methods known in the art as described by Barrett et al., 1998, Neuroscience, 85(4):1321-8, incorporated herein in its entirety. In another embodiment, cells are isolated by FACS using fluorogenic substrates of an enzyme transgenically expressed in a particular cell-type. The cells are isolated using methods known in the art as described by Blass-Kampmann et al., 1994, J. Neurosci. Res., 37(3):359-73, which is incorporated herein by reference in its entirerty.

The invention also provides methods for isolating cells from primary culture cells. Using methods known in the art, whole animal sorting (WACS) is accomplished whereby live cells derived from animals harboring a lacZ transgene are purified according to their level of beta-galactosidase expression with a fluorogenic beta-galactosidase substrate and FACS. See Krasnow et al., 1991, Science 251:81-5, which is incorporated herein by reference in its entirety.

In other embodiments of the invention, cells are isolated by FACS using fluorescent, vital dyes to retrograde label cells with fluorescent tracers. Cells are isolated using the methods described by St. John and Stephens, 1992, Dev. Biol. 151(1):154-65, Martinou et al., 1992, Neuron 8(4):737-44. Clendening and Hume, 1990, J. Neurosci. 10(12):3992-4005 and Martinou et al., 1989, J. Neurosci, 9(10):3645-56, which are incorporated herein by reference in their entireties.

In yet other embodiments of the invention, cells are isolated by FACS using fluorescent-conjugated lectins in retrograde labeled cells. The cells are isolated using the methods described in Schaffner et al., 1987, J. Neurosci., 7(10):3088-104 and Armson and Bennett, 1983, Neurosci. Lett., 38(2):181-6, which are incorporated herein by reference in their entireties.

In certain embodiments of the invention, cells are isolated by panning on antibodies against cell surface markers. In preferred embodiments, the antibody is a monoclonal antibody. Cells are isolated and characterized using methods known in the art described by Camu and Henderson, 1992, J. Neurosci. Methods 44(1):59-79, Kashiwagi et al., 2000, 41(1):2373-7, Brocco and Panzetta, 1997, 75(1):15-20, Tanaka et al., 1997. Dev. Neurosci. 19(1):106-11, and Barres et al., 1988, Neuron 1(9):791-803, which are incorporated herein by reference in their entireties.

In another embodiment, cells are isolated using laser capture microdissection (LCM). Methods for laser capture microdissection of the nervous system are well known in the art. See, e.g., Emmert-Buck et al., 1996, Science 274, 998-1001; Luo, et al., 1999, Nature Med. 5(1), 117-122; Ohyama et al., 2000, Biotechniques 29(3):530-36; Murakami et al., 2000, Kidney Int. 58(3),1346-53; Goldsworthy et al., 1999, Mol. Carcinog. 25(2): 86-91; Fend et al., 1999, Am. J. Pathol. 154(1):61-66); Schutze et al 1998, Nat. Biotechnol. Aug; 16(8):737-42.

In a specific embodiment, a collection of transgenic mouse lines of the invention is used to isolate neurons in the arcuate nucleus of the hypothalamus that regulate feeding behavior.

4.8. Uses of Transgenic Animal Collections

The collection of transgenic animal lines of the invention may be used for the identification and isolation of pure populations of particular classes of cells, which then may be used for pharmacological, behavioral, electrophysiological, gene expression, drug discovery, target validation assays, etc.

In certain embodiments, cells expressing the system gene coding sequences are detected in vivo in the transgenic animal, or in explanted tissue or tissue slices from the transgenic animal, to analyze the population of cells marked by the expression of the system gene coding sequences. In particular, the population of cells can be examined in transgenic animals treated or untreated with a compound of interest or other treatment, e.g., surgical treatment. The cells are detected by methods known in the art depending upon the marker gene used (see Section 4.6, above). In a particular embodiment, the system gene coding sequences encode or promote the production of an agent that enhances the contrast of the cells expressing the system gene coding sequences and such cells are detected by MRI.

Additionally, the transgenic animals may be bred to existing disease model animals or treated pharmacogically or surgically, or by any other means, to create a disease state in the transgenic animal. The marked population of cells can then be compared in the animal having and not having the disease state. Additionally, treatments for the disease may be evaluated by administering the treatment (e.g., a candidate compound) to the transgenic mice of the invention that have been bred to a disease state or a disease model otherwise induced in the transgenic mice and then detecting the marked population of cells. Changes in the marked population of cells are assayed, for example, for morphological, physiological or electrophysiological changes, changes in gene expression, protein-protein interactions, protein profile in response to the treatment is an indication of efficacy or toxicity, etc., of the treatment.

In other preferred embodiments, cells expressing the system gene are isolated from the transgenic animal using methods known in the art (for example, those methods described in Section 4.7, infra) for analysis or for culture of the cells and subsequent analysis. In certain embodiments, the transgenic animal may be subjected to a treatment (for example, a surgical treatment or administered a candidate compound of interest) prior to isolation of the cells. In other embodiments, the transgenic animal may be bred to a disease model or a disease state induced in the transgenic animal, for example, by surgical or pharmacological manipulation, prior to isolation of the cells. Additionally, that transgenic animal in which the disease state is induced may be subjected to treatments prior to isolation of the cells. The cells can then be directly analyzed as discussed below or can be cultured and subjected to additional treatments, for example, exposed to a candidate compound of interest.

Once isolated, the populations of cells can be analyzed by any method known in the art. In one aspect of the invention, the gene expression profile of the cells is analyzed using any number of methods known in the art, for example but not by way of limitation, by isolating the mRNA from the isolated cells and then hybridizing the cells to a microarray to identify the genes which are or are not expressed in the isolated cells. Gene expression in cells treated and not treated with a compound of interest or in cells from animals treated or untreated with a particular treatment may be compared. In addition, mRNA from the isolated cells may also be analyzed, for example by northern blot analysis, PCR, RNase protection, etc., for the presence of mRNAs encoding certain protein products and for changes in the presence or levels of these mRNAs depending on the treatment of the cells. In another aspect, mRNA from the isolated cells may be used to produce a cDNA library and, in fact, a collection of such cell type specific cDNA libraries may be generated from different populations of isolated cells. Such cDNA libraries are useful to analyze gene expression, isolate and identify cell type-specific genes, splice variants and non-coding RNAs. In another aspect, such cell type specific libraries prepared from cells isolated from treated and untreated transgenic animals of the invention or from transgenic animals of the invention having and not having a disease state can be used, for example in subtractive hybridization procedures, to identify genes -expressed at higher or lower levels in response to a particular treatment or in a disease state as compared to untreated transgenic animals. Data from such analyses may be used to generate a database of gene expression analysis for different populations of cells in the animal or in particular tissues or anatomical regions, for example, in the brain. Using such a database together with bioinformatics tools, such as hierarchical and non-hierarchical clustering analysis and pricipal components analysis, cells are “fingerprinted” for particular indications from healthy and disease-model animals or tissues.

In yet another embodiment, specific cells or cell populations isolated from the collection are analyzed for specific protein-protein interactions or an entire protein profile using proteomics methods known in the art, for example, chromatography, mass spectroscopy, 2D gel analysis, etc.

In yet another embodiment, specific cells or cell populations isolated from the collection are used as targets for expression cloning studies, for example, to identify the ligand of a receptor known to be present on a particular type of cell. Additionally, the isolated cells can be used to express a protein of unknown function to identify a function for that protein.

Other types of assays may be used to analyze the cell population either in vivo, in explanted or sectioned tissue or in the isolated cells, for example, to monitor the response of the cells to a certain treatment or candidate compound. The cells may be monitored, for example, but not by way of limitation, for changes in electrophysiology, physiology (for example, changes in physiological parameters of cells, such as intracellular or extracellular calcium or other ion concentration, change in pH, change in the presence or amount of second messengers, cell morphology, cell viability, indicators of apoptosis, secretion of secreted factors, cell replication, contact inhibition, etc.), morphology, etc.

In a particular embodiment, a subpopulation of cells in the isolated cells is identified and/or gene expression analyzed using the methods of Serafini et al., PCT Publication WO 99/29873 which is hereby incorporated by reference in its entirety.

### 5. EXAMPLE 1

This example describes the creation of a transgenic animal line of the invention.

5.1. Isolation and Initial Mapping of BACs

A BAC clone is isolated with either a unique cDNA or genomic DNA probe from BAC libraries for various species, (in the form of high density BAC colony DNA membrane). The BAC library is screened and positive clones are obtained, and the BACs for specific genes of interest are confirmed and mapped,. as described in detail below.

Probes

Overlapping oligonucleotide (“overgo”) probes are highly useful for large-scale physical mapping and whenever sequence is available from which to design a probe for hybridization purposes. In particular, the short length of the overgo probe is advantageous when there is limited available sequence known from which to design the probe. In addition, overgo probes obviate the need to clone and characterize cDNA fragments, which traditionally have been used as hybridization probes. Overgo probes can be used for identifying homologous sequences on DNA macroarrays printed on nylon membranes (i. e., BAC DNA macroarrays) or for Southern blot analysis. This technique can be extended to any hybridization-based gene screening approach. The following protocol describes a method for generating hybridization probes of high specific activity and specificity when sequence data is available. The method is used for identifying homologous DNA sequences in arrays of BAC library clones.

Design of Overgo Probes

Overgo probes are designed through a multistep process designed to ensure several important qualities:

(1) Overgos are gene-specific so that they do not hybridize to each other (when probes are pooled) or to sequences in the genome other than those that belong to the gene of interest.

(2) Probes are designed with similar GC contents. This allows probes to be labeled to similar specific activities and to hybridize with similar efficiencies, thus enabling a probe pooling strategy that is essential for high throughput screening of BAC library macroarrays.

The starting point for overgo design is to obtain sequence information for the gene of interest. The software packages required for overgo design require this sequence to be in FASTA format (http://www.ncbi.nlm.nih.gov/BLAST/fasta.html). The sequence used for overgo design should genomic, but cDNA sequences have been used successfully. To design a probe, a region of approximately 500 bp is selected. The 500 bp region should flank the gene's start codon (ATG) for probe design. This strategy gives a high probability of identifying BACs containing the 5′ end of the gene (and presumably many or all of the relevant transcriptional control elements. Selected sequences are screened for the presence of known murine DNA repeat sequences using the RepeatMasker program (http://ftp.genome.washington.edu/cgi-bin/ RepeatMasker). Oligonucleotides or “overgos” are then designed using Overgomaker (http//genome.wustl.edu/gsc/overgo/overgo.html). The overgo design program scans sequences and identifies two overlapping 24mers that have a balanced GC content, and an overall GC content between 40-60%. Once gene specific overgos have been designed, they are checked for uniqueness by using the BLAST program (NCBI) to compare them to the nr nucleic acid database (NCBI). Overgos that have significant BLAST scores for genes other than the gene of interest, i.e., could hybridize to genes other than the gene of interest, are redesigned.

Creation of Overgo Probes

To create an overgo probe, a pair of 24mer oligonucleotides overlapping at the 3′ ends by 8 base pairs are annealed to create double stranded DNA with 16 base pair overhangs. The resulting overhangs are filled in using Klenow fragment. Radionucleotides are incorporated during the fill-in process to label the resulting 40mer as it is synthesized. The overgo probe is then hybridized to immobilized BAC DNA. Following hybridization, the filter is washed to remove nonspecifically bound probe. Hybridization of specifically bound probe is visualized through autoradiography or phosphoimaging.

Materials

1. Target BAC clone DNA immobilized on nylon filters, for example,a macroarray of a BAC library, e.g., the CITB BAC library (Research Genetics) or the RPCI-23 library (BACPAC Resources, Children's Hospital Oakland Research Institute, Oakland, Calif.).

2. 10 μCi/μl [³²P]dATP (˜3000 Ci/mmol, 10 mCi/ml)

3. 10 μCi/μl [³²P]dCTP (˜3000 Ci/mmol, 10 mCi/ml)

4. Sephadex G-50 Microspin Column (e.g. ProbeQuant Spin Columns; Amersham Pharmacia Biotech)

5. 60° C. hybridization oven

6. SSC (sodium chloride/sodium citrate) 20×:

701.2 g NaCl

352 g NaCitrate

Add ddH₂O to make 4 L.

pH to 7.0 with 6M HCl

7. 10% SDS (sodium dodecyl sulfate):

100 g SDS/1 L dd H₂O

8. Church's hybridization buffer:

1 mM EDTA

7% SDS (use 99.9% pure SDS)

0.5 M Sodium phosphate

1 M Sodium phosphate, pH 7.2:

268 g Na2HPO4; 7H₂O in 1700 ml ddH₂O

Add 8 ml 85% H₃PO₄ and ddH₂O to make 2000 ml.

9. 0.5M EDTA, pH 8.0:

To make 500 ml:

93 g EDTA (disodium dihydrate) in 400 ml ddH₂O.

pH to 8.0 with 6M NaOH and add ddH₂O to make 500 ml.

To make 4000 ml:

To 2000 ml 1M sodium phosphate, add 1200 ml ddH₂O, 8 ml 0.5M EDTA and 280 g SDS.

Heat and stir until SDS is dissolved (approximately 1 hr.).

Add ddH₂O to bring volume to 4000 ml.

Warm to 60° C. before using.

10. Wash Buffer B: 1% SDS, 40 mM NaPO₄, 1 mM EDTA, pH 8.0

4×: 48 ml 0.5M EDTA

240 g SDS

960 ml IM NaHPO₄, pH 7.2

Add ddH₂O to make 6 L.

11. Wash Buffer 2: 1.5×SSC, 0.1% SDS

1125 ml 20×SSC

150 ml 10% SDS

Add ddH₂O to make 15 L.

12. Wash Buffer 3: 0.5×SSC, 0.1% SDS

375 ml 20×SSC

150 ml 10% SDS

Add ddH₂O to make 15 L.

13. 2% BSA: 200 mg BSA/10 ml ddH₂O

14. Stripping Buffer: 0.1×SSC, 0.1% SDS

10 ml 20×SSC

20 ml 10% SDS

Add ddH₂O to make 2 L.

15. Overgo Labeling Buffer (OLB)

Solution O:

125 mM MgCl₂

1.25 M Tris-HCl, pH 8.0

15.1 g Tris-base

2.54 g MgCl₂.6H₂O

Add ddH₂O to make 100 ml.

Solution A:

1 ml Solution O

18 μl 2-mercaptoethanol

5 μl 0.1 M dGTP

5 μl 0.1 M dTTP

Store up to 1 year at −80° C.

Solution B:

2 M HEPES-NaOH, pH 6.6

2.6 g HEPES to 5 ml ddH₂O

pH to 6.6 with approximately 2 drops 6M NaOH

Store up to 1 year at room temperature

Solution C:

3 mM Tris-HCl pH 7.4/0.2 mM Na₂EDTA

36 mg Tris-base

7 mg EDTA

Add ddH₂O to make 100 ml.

pH to 7.4 with 1M NaOH

Store up to 1 year at room temperature.

OLB:

A:B:C, in a 2:5:3 ratio

1 ml Solution A

2.5 ml Solution B

1.5 ml Solution C

Store in 0.5 ml aliquots at −20° C. for up to 3 months.

Methods

Annealing Oligonucleotides to Generate a Overhang

Step 1: combine 1.0 μl of partially complementary 10 μM oligos (1.0 μl forward primer+1.0 μl reverse primer) with 3.5 μl ddH₂O (10 pmol each oligo/reaction) to either a tube or microtiter plate well.

Step 2: Cap each tube or microtiter well and heat the paired oligonucleotides for 5 min at 80° C. to denature the oligonucleotides.

Step 3: Incubate the labeling reactions for 10 min at 37° C. to form overhangs.

Step 4: Store the annealed oligonucleotides on ice until they are labeled. If the labeling step is not done within 1 hour of annealing the oligonucleotides, repeat steps 2 and 3 before proceeding.

A thermocycler can be programmed to perform steps 2 through 4.

Overgo Labeling

Overgo probes can be labeled and hybridized using methods well-known in the art, for example, using the protocols described in Ross et al., 1999, Screening Large-Insert Libraries by Hybridization, In Current Protocols in Human Genetics, eds. N. C. Dracopoli, J. L. Haines, B. R. Korf, D. T. Moir, C. C. Morton, C. E. Seidman, J. G. Seidman, D. R. Smith. pp. 5.6.1-5.6.52 John Wiley and Sons, New York; incorporated herein by reference in its entirety.

The following protocol is modified after Ross et al., supra. Prepare a master mix containing the following reagents for each overgo probe to be labeled:

0.5 μl 2% BSA

2.0 μl overgo labeling buffer

0.5 μl [³²P]dATP

0.5 μl [³²P]dCTP

1.0 μl 2U/μl Klenow fragment

When making a master mix to label a number of overgo probes, prepare more than needed to ensure that there will be sufficient mix to account for small losses when transferring. An extra 10% is usually sufficient.

This protocol uses both [³²P]dATP and [³²P]dCTP for labeling. This is recommended; however, the composition of the dNTP mix in the overgo labeling buffer can be altered to allow different labeled deoxynucleotides to be used.

Pipet 4.5 μl of overgo labeling master mix to each of the annealed oligonucleotide pairs from step 4.

Incubate labeling reactions at room temperature for 1 hour.

Removal of Unincorporated Nucleotides

Remove unincorporated nucleotides using a Sephadex G-50 microspin column following the manufacturers protocol. If probes will be pooled, multiple labeling reactions can be combined and processed simultaneously as long as the total volume specified by the manufacturer is not exceeded.

Checking Incorporation

The following method can be used as a quick measure of the success of the labeling reaction.

Dilute the probes 1:100 (1 μl probe+99 μl H₂O), and use 1 μl of diluted probe for scintillation counting. For optimal hybridization, the probe specific activity should be approximately 5×10⁵ cpm/ml.

5.1.1. BAC Screening

BACs containing specific genes of interest are identified by using ³²P labeled overgo probes, as described above, to probe nylon membranes onto which BAC-containing bacterial colonies have been spotted. Traditionally, BAC screening is accomplished by hybridizing a single probe to BAC library filters, and identifying positive clones for that single gene. The use of overgo probes makes it possible to adopt a probe pooling strategy that permits higher throughput while using fewer library filters. In this strategy, probes are arrayed into a two-dimensional matrix (i.e., 5×5 or 6×6). Then probes are combined into row and column pools (e.g., 10 pools total for a 5×5 array). Each probe pool is hybridized to a single copy of the BAC library filters (10 separate hybridizations) e.g., the CITB or RPCI-23 BAC library filters.

Following hybridization and autoradiography or phosphoimaging, clones hybridizing to each probe pool ( 4-5 probes) are manually identified. Assignment of positive clones to individual probes is done by pairwise comparisons between each row and each column. The intersection of each row pool and column pool defines a single probe within the probe array. Thus, all positive clones that are shared in common by a specific row pool and a specific column pool are known to hybridize to the probe defined by the unique intersection between the row and column. Deconvolution of hybridization data to assign positive clones to specific probes in the probe array is done manually, or by using an excel-based visual basic program.

Using this strategy increases screening efficiency, and throughput, while decreasing the number of library filters required. For example, without probe pooling, hybridizing 25 probes would require 25 sets of library filters. In contrast, a 5×5 probe array requires only 10 probe pools, thus 10 hybridizations and 10 filter sets. This approach can also be extended using 3 dimensional probe arrays. For example, a 3×3×3 array allows for identification of 27 genes and only requires 9 hybridization experiments.

Hybridization of Overgo Probe to Nylon Filter

The nylon filters are prehybridized by wetting with 60° C. Church's hybridization buffer and rolling the filters into a hybridization bottle filled halfway or approximately 150 ml of 60° C. Church's hybridization buffer. All of the filters are rolled in the same direction (DNA and writing side up), with a nylon mesh spacer in between each and on top, and the bottle is placed in the oven to keep them rolled. The rotation speed is set to 8-9 speed. The filter is incubated at 60° C. for at least 4 hours the first time (1-2 hours for subsequent prehybridizations of the same filters).

Following prehybridization of the filters, labeled probes are denatured by heating to 100° C. for 10 min and then placed on slushy ice for >2 min.

The Church's hybridization buffer is replaced before adding probes if the filter is used for the first time. Filters are incubated with the probe at 60° C. overnight. The rotation speed is set to 8-9 speed.

The next day, the Church's hybridization buffer is drained from the bottle and 100 ml Washing Buffer B pre-heated to 60° C. is added. The hybridization bottle is returned to the incubation oven for 30 min. The rotation speed is set to 8-9 speed. Church's hybridization buffer and Washing Buffer B are radioactive and must be disposed of in a liquid radioactive waste container.

Washing Buffer B is drained from the bottle and 80 ml Washing Buffer 2 pre-heated to 60° C. is added. The hybridization bottle is returned to the incubation oven for 20 min. The rotation speed is set to 8-9 speed.

Washing Buffer 2 is drained from the bottle and 80 ml Washing Buffer 2 pre-heated to 60° C. is added. The hybridization bottle is returned to the incubation oven for 20 min. The rotation speed is set to 8-9 speed.

Filters are removed from the hybridization bottles and washed in a shaking bath for 5 min. at 60° C. with 2.5 L Washing Buffer 3, shaking slowly, without overwashing.

Filters are soaked in Church's hybridization buffer.

Filters are removed from the bath, spacers are set aside, and placed in individual Kapak, 10″×12,″ Sealpak pouches. All air bubbles are removed by rolling with a glass pipette. The pouches are sealed and checked for leaks. A damp tissue removes any remaining solution on the outside of the bag.

Each filter is placed in an autoradiograph cassette at room temperature with an intensifying screen. An overnight exposure at room temperature is usually adequate. Alternatively, the data can be collected using a phosphorimager if available.

Probes may be stripped from the filters (not routinely done) by washing in 1.5 L 70° C. Stripping Buffer for 30 min. Counts are checked with a survey meter to verify the efficacy of stripping procedure. This is repeated for an additional 10 min. if necessary. Filters should not be overstripped. Overstripping removes BAC DNA and reduces the life of the filters.

Stripping may be incomplete, so it is necessary to autoradiograph the stripped filter if residual probe may confuse subsequent hybridization results.

Identification and Confirmation of Clones

The CTIB and RPCI-23 BAC library filters come as sets of 5-10 filters that have 30-50,000 clones spotted in duplicate on each filter. Following autoradiography, positive clones appear as small dark spots. Because clones are spotted in duplicate, true positives always appear as twin spots within a subdivision of the macroarray. Using templates and positioning aids provided by the filter manufacturer, unique clone identities are obtained for each positive clone. Once the identities of clones for each probe have been identified, they are ordered from BACPAC Resources (http://www.chori.org/bacpac/) or Research Genetics (http://www.resgen.com/). To confirm that clones have been correctly identified, each clones is rescreened by PCR using gene specific primers that amplify a portion of the 5′ or the 3′ end of the gene. In some cases, clones are tested for the presence of both 5′ and 3′ end amplicons. Other BAC libraries, including those from non-commercial sources may be used. Clones may be identified using the hybridization method described above to filters with arrayed clones having an identifiable location on the filter so that the corresponding BAC of any positive spots can be obtained.

5.1.2. Mapping of BACs

Once BACs for a gene of interest have been identified, the position of the gene within the BAC must be determined. To design reporter systems that faithfully reproduce the normal expression pattern of the gene of interest, it is critical that the BAC contain the necessary transcriptional control elements required for wild-type expression. As a first approximation, it can be hypothesized that if the gene lies near the center of a BAC that is 150-200 kb in length, then the BAC will likely contain the control elements required to reproduce the wild type expression pattern. Thus, it becomes critical to use methods for approximating the position of the gene of interest within the BAC.

Fingerprinting of BACs

Fingerprinting methods rely on genome mapping technology to assemble BACs containing the gene of interest into a contig, i.e., a continuous set of overlapping clones. Once a contig has been assembled, it is straightforward to identify 1 or 2 center clones in the contig. Since all clones in the contig hybridize to the 5′ end of the gene (because the probe sequence is designed to hybridize at or near the start codon of the gene's coding sequence), the center clones of the contig should have the gene in the central-most position.

A mouse BAC library, e.g., a RPCI-23 BAC library, can be fingerprinted using the methods of Soderlund et al. (2000, Genome Res. 10(11):1772-87; incorporated herein by reference in its entirety). BACs are fingerprinted using HindIll digestion digests. Digests are run out on 1% agarose gels, stained with sybr green (Molecular Probes) and then visualized on a Typhoon fluoroimager (Amersham Pharmacia). Gel image data is acquired using the “IMAGE” program (Sanger Center; http://www.sanger.ac.uk/). Data from “IMAGE” is then passed along to the analysis program “FPC” (fingerpring contig)(Sanger Center; http://www.sanger.ac.uk/). Using FPC, the data from a publicly available genome database can be queried to determine if the insert of a particular BAC has been fingerprinted and contigged. BAC fingerprint information has been generated by the University of British Columbia Genome Mapping Project (http://www.bcgsc.bc.ca/projects/mouse_mapping) and can be used for assembling BAC contigs. Preferably, contig information from publicly available databases is used to select clones for BAC modification as described above.

If an existing contig cannot be identified from publicly available data, three alternative strategies are used to determine which BAC is the best candidate for recombination:

1) Restriction Mapping

In the first step of the BAC recombination process, the shuttle vector (containing the homology region and the system gene coding sequences) integrates into the BAC to form the cointegrate. This process introduces a unique Asc-1 restriction site into the BAC at the site of cointegration. It is possible to map the position of this site, by first cutting the cointegrate with Not-1, which releases the BAC insert (approx 150-200 kb) from the BAC vector. Subsequent digestion with Asc-1 (which cuts very rarely in mammalian genomes), should cleave the BAC insert once, yielding two fragments. The fragment sizes can be accurately resolved using the CHEF gel mapping system (Bio-Rad). If the Asc-1 site is centrally located, then the insert should be cleaved into 2 nearly equal fragments of large size (˜75-100 kb each). If the Asc-1 site is located asymmetrically, then the homology region is not centered in the BAC, and thus is not a good candidate for transgenesis. Alternatively, if the size of the smaller fragment falls below a predetermined size (for example 50 kb), then that BAC should be ruled out as a candidate.

2) Fingerprinting

The fingerprinting method described above can also be used to generate additional fingerprint data. This data is used to generate contigs of currently uncontigged BACs from which center clones can be selected. In addition, this data can be combined with data from publicly available databases to generate novel contig information.

3) Alternative Mapping Method

If neither of the above methods is successful, then the following alternative mapping method is used to roughly localize a gene within a BAC clone. This method takes advantage of the fact that one end of the BAC genomic insert is linked to the SP6 promoter while the other end is linked to the T7 promoter. The alternative mapping method involves the following steps:

a) digestion with notl to release the BAC insert

b) digestion with another enzyme that cuts no more than 4-7 times in the BAC (in practice, we usually use several different enzymes). Digests are run out on a 0.7% agarose gel.

c) The gel is transferred to nylon, hybridized to alkaline phosphatase conjugated T7 oligo probe-develop and the blot is exposed according to the alternative mapping protocol described below. This step identifies that fragment containing the T7 end of the BAC insert.

d) Hybridization to alkaline phosphatase conjugated SP6 oligo probe. The blot is developed and exposed according to the alternative mapping protocol described below. This identifies fragment containing the SP6 end of the BAC insert.

e) Finally, the blot is hybridized to a gene specific probe. This identifies which fragment contains the gene.

If the gene-hybridizing fragment is different from the T7-or SP6- hybridizing fragments, and the latter two fragments are >30-50 kb, then these data show that the gene must be at least 30-50 kb away from the ends of the BAC, and thus is a likely candidate for transgenesis.

Alternative Mapping Protocol

1. Double digest each BAC DNA with four different rare cutters, together with Not1. Four 10 μl BAC DNA (out of 50 μl of alkalinelysis miniprep with 3 ml starting culture, roughly 10 ng pure BAC DNA) per digest are used.

| | |
|---|---|
| DNA | 4 μl |
| 10xB (NEB₄) | 1 μl |
| Cla1 | 0.3 μl |
| Not1 | 0.3 μl |
| ddH20 | 4.4 μl |
| | 10 μl |

1. A similar double digest is performed with SaclI/Not1 (with NEB buffer4), Sall/Not1 (Sal buffer), and Xho1/Not1 (buffer3). The digests are incubated for 2 hours at 37° C.

2. Loading dye is added (orange dye preferred for Typhoon fluoroimager) to the above entire reaction, and the reactions are loaded into a 0.7% agarose gel. The gel is run at 80V (for a 7×11 inch large gel) overnight.

3. The gel is stained with Vista green (1:10,000 dilution in TAE buffer) for 10-20 min and imaged on a Typhoon fluoroimager (Amersham Pharmacia) using the Fluorescence mode, 526 SP/Green (532 nm) setting. The gain and sensitivity are varied until the bands look dark but not saturated. Alternatively, bands can usually be visualized using standard ethidium bromide stain and visualized on a UV lightbox.

4. The gel is transferred into a large TUPPERWARE® container and depurinated with 0.125M HCL for 10 min, rinsed with ddH₂O once, then neutralized with 1.5M NaCl and 0.5M Tris-HCl (pH 7.5) for 30 min, and denatured with 0.5M NaOH and 1.5M NaCl for 30 min.

5. A capillary wet transfer in 0.5M NaOH and 1.5M NaCl is set up, following the instructions that come with the H+ nylon membrane, and the transfer runs overnight.

6. Next day, the well and lane positions are marked as well as the upper-right corner of the membrane (to keep track of which side is up and the location of the left and right lanes). The membrane is UV crosslinked.

Hybridization with Alkaline Phosphatase Conjugated T7 and SP6 Probes

T7 and SP7 hybridizations and exposures are done sequentially and are not to be performed together.

7. Wash buffer #1 and wash buffer #2 are prewarmed at 37° C.

8. The membrane is present with with ddH₂O. The membrane is prehybridized in hybridization buffer at 37° C. for 10 min. For the prehybridization and hybridization steps, exactly 50 μl of buffer is used per 1.0 cm² of membrane.

9. During the prehybridization step, the probe is diluted to a 2 nM final concentration in hybridization buffer. The volume is calculated as done in step 8. The correct probe concentration is crucial. The tubes containing these solutions are incubated at 37° C. during the prehybridization step.

10. After 10 min, all of the prehybridization buffer is removed and the hyb buffer containing probe is added. A hybridization step is done at 37° C. for 60 min.

The membrane should not dry out during the following wash, detection and film exposure.

11. 100 ml of prewarmed wash buffer 1 is poured into a container. The membrane is transferred into the container, swirled gently for 1 min. The buffer solution is poured out and 150-200 ml of wash buffer 1 is added and the membrane is washed for 10 min. with gentle agitation.

12. Buffer 1 is removed and prewarmed buffer 2 is added. Washes are done as in step 11 for another 10 min.

13. Washes with 2×SSC are done for 10 min at RT. The CSPD chemiluminescent substrate is removed from refrigeration and allowed to warm up to room temperature (RT).

14. The substrate buffer is prepared and 50 μl is used per 1.0 cm² of membrane.

15. The membrane is rinsed 2 times for 5 min. each in assay buffer. The membrane is incubated in substrate buffer inside heat-sealable bags at RT for 10 min. while manually agitating the bag to ensure that the membranes are covered with substrate buffer.

16. The membrane is removed from the substrate buffer and placed into a seal bag and exposed to KODAK® film (Eastman Kodak Co.) immediately.

Southern Hybridization with Gene Specific Probes

17. Probes are labeled using purified PCR product as a template with the Ready-Prime kit. The prehybridization and hybridization steps are carried out as in standard Southern blot hybridization. The membranes are exposed at room temperature or at 37° C. Alternatively, one can probe with a gene-specific overgo probe using the BAC screening protocol as described above.

Band Identification

18. The two blots are aligned with the original DNA gel. Positive bands are identified for T7/SP6 and the gene-specific probe.

1. Wash buffer 1:

2×SSC

1% (w/v) SDS

2. Wash buffer 2:

2×SSC

1% Triton-X-100

3. Substrate buffer:

5 ml of assay buffer

30 μl of CSPD chemiluminescent substrate

4. Hybridization buffer

1×SSC

1% SDS

0.5% BSA

0.5% PVP

0.01% NaN₃

5. Assay buffer

0.96 ml of DEA

0.1 ml of 1M MgCl₂

0.21 ml of 2M NaN₃

add ddH₂O to 80 ml

adjust to pH 10.0 with dilute HCl

add ddH₂O to make final 100 ml

5.2. BAC Recombination

Methods for introducing the system gene coding sequences into the characterizing gene sequences on the BAC through homologous recombination in bacteria are described below.

Cloning Homology Boxes

A homologous recombination shuttle vector is prepared in which the system gene is flanked at its 5′ and 3′ ends by characterizing gene sequences to allow for homologous recombination to occur between the exogenous gene carried by the shuttle vector and the characterizing gene sequences in the BAC cell. The additional flanking nucleic acid sequences are of sufficient length for successful homologous recombination with the characterizing gene on the BAC. Homology boxes are these regions of DNA and are used to direct site specific recombination between a shuttle vector and a BAC of interest. In one embodiment, the homologous regions comprise the 3′ portion of the characterizing gene. In preferred embodiments, the homologous regions comprise the 5′ portion of the characterizing gene, more preferably to target integration of the system gene coding sequences in frame with the ATC of the characterizing gene sequences. PCR is used for cloning a homology box from genomic DNA or BAC DNA. The homology box is cloned into the shuttle vector that is used for BAC recombination, as described below.

Design of PCR Primers

Using Primer3 program (Massachusetts Institute of Technology (http://www-genome.wi.mit.edu/cgi-bin/primer/primer3www.cgi), a AscI site is added in the 5′ forward primer and a Smal site is added in the 3′ reverse primer.

Using the Primer3 default temperature calculations, primers are designed so that they have Tₘs of 57-60° C. and so that the amplicons are between 300 and 500 bp in length.

If a 5′ UTR of the characterizing gene sequence is available, amplicons are designed against this sequence. If the 5′ UTR sequence is not available, then homology boxes are designed to include the 3′ UTR or the 3′ stop codon, or any other desired region of the characterizing gene.

PCR Reactions

PCR reactions are performed with the following reagents:

| | |
|---|---|
| 1.0 μl | Mouse genomic DNA or BAC having characterizing gene |
| | insert (500 ng/μl) |
| 1.0 μl | Forward primer 10 pmol/μl |
| 1.0 μl | Reverse primer 10 pmol/μl |
| 0.5 μl | 10 mM dNTP mix |
| 2.5 μl | 10XPCR buffer without MgCl₂ |
| 2.0 μl | 25 mM MgCl₂ |
| 0.125 μl | Taq AmpliGold (Perkin Elmer) |
| 15.875 μl | H₂O |

DNA template for PCR should be from the BAC to be modified, or genomic DNA from the same strain of mouse from which the BAC library was constructed. The homology boxes must be cloned from the same mouse strain as the BACs to be modified.

Preferably, Pfti DNA polymerase (Stratagene) is used. This reduces errors introduced into the amplified sequence via PCR with Taq polymerase.

Total volume is 25 μl.

1 drop (approximately 25 μl) of mineral oil is added to the PCR tubes before running the PCR reactions. PCR reactions are run on a thermal cycler using the following program:

| 1. | 95° C. | 10 min |
|---|---|---|
| 2. | 94° C. | 30 sec |
| 3. | 55-60° C. | 30 sec (annealing temperature is determined based on |
| | | the Tₘ of the primers used) |
| 4. | 72° C. | 45 min |
| 5. | go back to step 2 for 40 cycles. | |
| 6. | 72° C. | 10 min |
| 7. | 40° C. | hold |

Analysis of PCR Products

5 μl of the PCR reaction is run on 0.8% agarose gel. The bands are visualized with EtBr staining. Good PCR reactions produce a single product at the expected size. The yield of one PCR reaction is between 50 to 200 ng.

Cloning of the PCR Product

A TOPO-TA cloning kit (Invitrogen) is used to clone the PCR product. Ligation reactions are carried out at room temperature for 3 min with the following reagents:

| | | |
|---|---|---|
| 1 | μl | TOPO vector |
| 2-4 | μl | PCR reaction aliquot (depending on the yield of the reaction, |
| | | no purification is needed if only a single band is produced) |
| 0-2 | μl | ddH₂O |
| | | Optional: 1 μl salt solution (provided in the TOPO kit) |

2 μl of the ligation reaction is transformed into Top10 cells (Invitrogen) following the manufacturer's protocol.

A blue-white selection is used (spreading IPTG and X-gal solutions on the LB-Amp plates prior to plating the transformation mixture).

Analysis of TOPO-PCR Clones

Four white colonies are picked to start overnight 2 ml LB-Amp cultures. The DNA is extracted using a Qiagen miniprep kit. 2 μl (1/25) of the miniprep DNA is digested with EcoRI, which excises the inserts from the TOPO vectors. The identity of the clones is confirmed by sequence analysis using either T3 or T7 primers.

5.3. Homologous Recombination Between a Shuttle Vector and the BAC

Cointegrates of the BAC and a shuttle vector are prepared as follows. A shuttle vector containing IRES, GFP and the homology box, as described in PCT publication WO 01/05962, containing the system gene of interest is transformed into competent cells containing the BAC of interest by electroporation using the following protocol. A 40-μl aliquot of the BAC-containing competent cells is thawed on ice, the aliquot is mixed with 2 μl of DNA(0.5 μg /μl), and the mixture is placed on ice for 1 minute. Each sample is transferred to a cold 0.1 cm cuvette.

A Gene Pulser apparatus (Bio-Rad) is used to carry out the electroporation. The Gene Pulser apparatus is set to 25 μf, the voltage to 1.8 KV and pulse controller to 200 Ω.

1 ml SOC is added to each cuvette immediately after conducting the electroporation. The cells are resuspended. The cell suspension is transferred to a 17×100 mm polypropylene tube and incubated at 37° C. for one hour with shaking at 225 RPM.

The 1 ml culture is spun off and plated onto one chloramphenicol (Chl) (12.5 μg/ml) and ampicillin (Amp) (50 μg/ml) plate and incubated at 37° C. for 16-20 hours.

The colonies are picked and innoculated with 5 ml LB supplemented with Ch1(12.5 μg/ml) and Amp (50 μg/ml), incubate at 37° C. overnight. Miniprep DNA from 3 ml of cultures by alkaline lysis method. Cointegrates for each clone are identified by Southern blot. Using a homology box as a probe, the cointegrate can be identified by the appearance of an additional homology box that is introduced via the recombination process.

The resolved clones (i.e., clones in which the shuttle vector sequences have been removed, leaving the system gene sequences) from the modified BACs are screened and each colony of cointegrate from the Ch1/Amp plates is picked and used to innoculate 5 ml of LB+Ch1(12.5 μg/ml) and 6% sucrose, and incubated at 37° C. for 8 hours.

The culture is diluted 1:5000 and plated on the agar plate with Ch1 (12.5 μg/ml) and 6% sucrose and incubated at 37° C. overnight.

Five colonies per plate are picked and innoculated with 5 ml of LB+Ch1(12.5 μg/ml) only and incubated at 37° C. overnight. DNA from those cultures are miniprepped by alkaline lysis method known in the art. The resolved BACs are screened by Southern blot.

Construct Verification

To ensure that a cointegrate is formed properly, Southern blotting is performed to ensure that the first step of recombination has occurred properly. In addition, this step may be verified to determine that system gene sequences have been juxtaposed adjacent to the characterizing gene sequences.

After the shuttle vector is recombined into the BAC to form a cointegrate, the vector sequences are removed in a resolution step, as described in WO 01/05962, herein incorporated by reference in its entirety. After cointegrates are resolved, Southern blotting and PCR are used to confirm that resolution products are correct, i.e., the only modification to the BAC is that the reporter has been inserted at the homology box.

Identification and Purification of Recombinant BAC DNA

BAC DNA is purified as follows and is then used for pronuclear injection or other methods known in the art to create transgenic mice.

Maxiprep by Alkaline Lysis for BACs

1. 250 ml cultures are spun down overnight at 4000 rcf for 15 min.

2. The pellet is resuspended in P1 buffer (RNase-free), 20 ml, pipetting.

3. Cells are lysed for 4-5 min in P2 buffer, 40 ml, mix briefly by inversion or swirling.

4. 20 ml cold P3 buffer is added, mixed briefly, and incubated on ice for 10 min.

5. The pellet is spun down on a swing bucket rotor at maximum speed for 20 min.

6. The supernatant is filtered through four layers of cheesecloth into clean 250 ml tubes.

7. 2×volume of 95% EtOH is added and the suspension is spun on a swing bucket rotor at maximum speed for 20 min.

8. The pellet is resuspended.

9. DNA is precipitated with 5 ml 5M LiCl (final conc. 2.5M), on ice for 10 min.

10. Precipitate is spun at 4000 rpm for 20 min. by a Sorval tabletop centrifuge.

11. The supernatant is transferred to fresh 50 ml Falcon tubes.

12. 1×volume isopropanol is added.

13. The precipitate is spun at 4000 rpm for 20 min on Sorval tabletop centrifuge.

14. The pellet is washed with 1 ml 70% EtOH.

15. The DNA is resuspended in 500λ TE.

16. 5λ RNase, DNAse-free. (Roche) is added to the DNA.

17. RNase A is added to a final concentration of 25 μg/ml. (Qiagen).

18. The DNA is incubated for 1 hr at 37° C.

19. The DNA is phenol extracted 10 min on ADAMS™ Nutator Mixer (BD Diagnostic Systems).

20. 250 μl NH₄OAc+750 μl isopropanol is added.

21. Precipitate is spun for 10 min. at maximum speed on Eppendorf at 4° C.

22. The pellet is resuspended in 50 μl TE

The DNA is purified for injection by either treatment with plasmid safe endonuclease (Epicenter Technologies) or by gel filtration using Sephacryl S-500 column or CL4b Sepharose column (both from Amersham Pharmacia Biotech).

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

I claim:

1. A collection of lines of transgenic animals comprising two or more of said lines of transgenic animals wherein each of said transgenic animals comprises a transgene, said transgene comprising (a) first sequences coding for a selectable or detectable marker protein; and (b) regulatory sequences of a characterizing gene corresponding to an endogenous gene or ortholog of an endogenous gene operably linked to said first sequences such that said first sequences are expressed in said transgenic animal with an expression pattern that is substantially the same as the expression pattern of said endogenous gene in a non-transgenic animal or anatomical region thereof wherein the characterizing gene is different for each of said transgenic animals, and wherein said transgene is present in the genome at a site other than where the endogenous gene is located.

2. The collection of lines of transgenic animals of claim 1 wherein said transgenic animals are transgenic mice.

3. The collection of lines of transgenic animals of claim 1 which comprises ten or more lines of transgenic animals.

4. The collection of lines of transgenic animals of claim 1 which comprises fifty or more lines of transgenic animals.

5. The collection of lines of transgenic animals of claim 1 wherein said transgene further comprises a coding sequence of said characterizing gene.

6. The collection of lines of transgenic animals of claim 5 wherein said first sequences are inserted or replace sequences 5′ of said coding sequence of said characterizing gene.

7. The collection of lines of transgenic animals of claim 1 wherein said first sequences are operably linked to an IRES sequence that is not operably linked to a coding sequence of said characterizing gene.

8. The collection of lines of transgenic animals of claim 5 wherein said first sequences are fused in frame to the ATG start codon of said coding sequence of said characterizing gene.

9. The collection of lines of transgenic animals of claim 1 wherein said characterizing gene is not functionally expressed from said transgene.

10. The collection of lines of transgenic animals of claim 1 wherein said first sequences encode a detectable enzyme.

11. The collection of lines of transgenic animals of claim 10 wherein said detectable enzyme is β-lactamase.

12. The collection of lines of transgenic animals of claim 1 wherein said first sequences encode a fluorescent protein.

13. The collection of lines of transgenic animals of claim 12 wherein fluorescent protein is a green fluorescent protein (GFP).

14. The collection of lines of transgenic animals of claim 1 wherein each said endogenous gene is expressed in the same tissue.

15. The collection of lines of transgenic animals of claim 1 wherein each said endogenous gene is specifically expressed in a subset of neurons.

16. The collection of lines of trans genic animals of claim 1 wherein each said endogenous gene is endogenously expressed in neuronal cells.

17. The collection of lines of transgenic animals of claim 1 wherein each of said endogenous genes endogenously expresses a protein product that is a part of an adrenergic or noradrenergic neurotransmitter pathway, a cholinergic neurotransmitter pathway, a dopaminergic neurotransmitter pathway, a GABAergic neurotransmitter pathway, a glutaminergic neurotransmitter pathway, a glycinergic neurotransmitter pathway, a histaminergic neurotransmitter pathway, a neuropeptidergic neurotransmitter pathway, a serotonergic neurotransmitter pathway, or the sonic hedgehog signaling pathway, is a nucleotide receptor, an ion channel, a marker of undifferentiated or not fully differentiated nerve cells, a calcium binding protein, or a neurotrophic factor receptor.

18. The collection of lines of transgenic animals of claim 1 wherein all of said endogenous genes are functionally related.

19. The collection of lines of transgenic animals of claim 1 wherein each of said endogenous genes is implicated in the same physiological or disease state.

20. The collection of lines of transgenic animals of claim 19 wherein the physiological or disease state is a neurological or psychiatric disease.

21. The collection of lines of transgenic animals of claim 20 wherein the neurological or psychiatric disease is schizophrenia, schizotypal personality disorder, psychosis, a schizoaffective disorder manic type disorder, a bipolar affective disorder, a bipolar affective (mood) disorder with hypomania and major depression (BP-II), a unipolar affective disorder, unipolar major depressive disorder, dysthymic disorder, a obsessive-compulsive disorder, a phobia, a panic disorder, a generalized anxiety disorder, a somatization disorder, hypochondriasis, or an attention deficit disorder.

22. The collection of lines of transgenic animals of claim 1 wherein each of said endogenous genes is implicated in the same physiological or behavioral response.

23. The collection of lines of transgenic animals of claim 22 wherein said physiological or behavioral response is pain, sleeping, feeding, fasting, sexual behavior or aggression.

24. The collection of lines of transgenic animals of claim 1 wherein each of said endogenous genes is expressed in neuronal cells involved in regulation of feeding behavior.

25. The collection of lines of transgenic animals of claim 1 wherein each of said endogenous genes is expressed in a different tissue.

26. The collection of lines of transgenic animals of claim 1 wherein each of said endogenous genes is implicated in a different physiological or disease state.

27. The collection of lines of transgenic animals of claim 1 wherein each of said endogenous genes is implicated in a different physiological or behavioral response.

28. A collection of lines of transgenic animals comprising two or more of said lines of transgenic animals wherein each of said transgenic animals comprises a transgene, said transgene comprising (a) first sequences coding for an activator or repressor of expression of second sequences encoding a detectable or selectable marker; and (b) regulatory sequences of a characterizing gene corresponding to an endogenous gene or ortholog of an endogenous gene operably linked to said first sequences such that said first sequences are expressed in said transgenic animal with an expression pattern that is substantially the same as the expression pattern of said endogenous gene in a non-transgenic animal or anatomical region thereof, wherein the characterizing gene is different for each of said transgenic animals, and wherein said transgene is present in the genome at a site other than where the endogenous gene is located; each of said transgenic animals also comprising said second sequences operably linked to an expression control element activatable or repressible by said activator or repressor.

29. The collection of lines of transgenic animals of claim 28 wherein said second sequences are contained within said transgene.

30. The collection of lines of transgenic animals of claim 28 wherein said second sequences are not contained within said transgene.

31. The collection of lines of transgenic animals of claim 30 wherein said second sequences are introduced into the genome of said transgenic animal by breeding.

32. A method of making a collection of lines of transgenic animals said method comprising
(a) introducing into the genome of a founder animal a transgene comprising (i) first sequences coding for a selectable or detectable marker protein and (ii) regulatory sequences of a characterizing gene corresponding to an endogenous gene or ortholog of an endogenous gene operably linked to said first sequences such that said first sequences are expressed in said transgenic animal with an expression pattern that is substantially the same as the expression pattern of said endogenous gene in a non-transgenic animal or anatomical region thereof;
(b) breeding said founder animal to produce a line of transgenic animals; and
(c) repeating steps (a) and (b) one or more times, each time with a different characterizing gene to generate one or more additional lines of transgenic animals,
thereby generating said collection of lines of transgenic animals.

33. The method of claim 32 wherein said transgenic animals are transgenic mice.

34. The method of claim 32 wherein said collection comprises ten or more lines of transgenic animals.

35. The method of claim 32 wherein said collection comprises fifty or more lines of transgenic animals.

36. The method of claim 32 wherein said transgene further comprises a coding sequence of said characterizing gene.

37. The method of claim 36 wherein said first sequences are inserted or replace sequences 5′ of said coding sequence of said characterizing gene.

38. The method of claim 32 wherein said first sequences are operably linked to an IRES sequence that is not operably linked to a coding sequence of said characterizing gene.

39. The method of claim 36 wherein said first sequences are fused in frame to the ATG start codon of said coding sequence of said characterizing gene.

40. The method of claim 32 wherein said characterizing gene is not functionally expressed from said transgene.

41. The method of claim 32 wherein said first sequences encode a detectable enzyme.

42. The method of claim 41 wherein said detectable enzyme is β-lactamase.

43. The method of claim 32 wherein said first sequences encode a fluorescent protein.

44. The method of claim 43 wherein fluorescent protein is a GFP.

45. The method of claim 32 wherein each said endogenous gene is expressed in the same tissue.

46. The method of claim 32 wherein each said endogenous gene is specifically expressed in a subset of neurons.

47. The method of claim 32 wherein each said endogenous gene is endogenously expressed in neuronal cells.

48. The method of claim 32 wherein each of said endogenous genes endogenously expresses a protein product that is a part of an adrenergic or noradrenergic neurotransmiter pathway, a cholinergic neurotransmitter pathway, a dopaminergic neurotransmitter pathway, a GABAergic neurotransmitter pathway, a glutaminergic neurotransmitter pathway, a glycinergic neurotransmitter pathway, a histaminergic neurotransmitter pathway, a neuropeptidergic neurotransmitter pathway, a serotonergic neurotransmitter pathway, or the sonic hedgehog signaling pathway, is a nucleotide receptor, an ion channel, a marker of undifferentiated or not fully differentiated nerve cells, a calcium binding protein, or a neurotrophic factor receptor.

49. The method of claim 32 wherein all of said endogenous genes are functionally related.

50. The method of claim 32 wherein each of said endogenous genes is implicated in the same physiological or disease state.

51. The method of claim 50 wherein the physiological or disease state is a neurological or psychiatric disease.

52. The method of claim 51 wherein the neurological or psychiatric disease is schizophrenia, schizotypal personality disorder, psychosis, a schizoaffective disorder manic type disorder, a bipolar affective disorder, a bipolar affective (mood) disorder with hypomania and major depression (BP-II), a unipolar affective disorder, unipolar major depressive disorder, dysthymic disorder, a obsessive-compulsive disorder, a phobia, a panic disorder, a generalized anxiety disorder, a somatization disorder, hypochondriasis, or an attention deficit disorder.

53. The method of claim 32 wherein each of said endogenous genes is implicated in the same physiological or behavioral response.

54. The method of claim 53 wherein said physiological or behavioral response is pain, sleeping, feeding, fasting, sexual behavior or aggression.

55. The method of claim 32 wherein each of said endogenous genes is expressed in neuronal cells involved in regulation of feeding behavior.

56. The method of claim 32 wherein each of said endogenous genes is expressed in a different tissue.

57. The method of claim 32 wherein each of said endogenous genes is implicated in a different physiological or disease state.

58. The method of claim 32 wherein each of said endogenous genes is implicated in a different physiological or behavioral response.

59. The method of claim 32 wherein prior to introduction into said founder animal said transgene is contained within a bacterial artificial chromosome (BAC).

60. The method of claim 32 wherein said transgene is introduced by pronuclear injection.

61. A method of making a collection of lines of transgenic animals, said method comprising
(a) introducing into the genome of a founder animal a transgene comprising (i) first sequences coding for an activator or repressor of expression of second sequences encoding a detectable or selectable marker and (ii) regulatory sequences of a characterizing gene corresponding to an endogenous gene or ortholog of an endogenous gene operably linked to said first sequences such that said first sequences are expressed in said transgenic animal with an expression pattern that is substantially the same as the expression pattern of said endogenous gene in a non-transgenic animal or anatomical region thereof;
(b) breeding said founder animal to produce a line of transgenic animals; and
(c) repeating steps (a) and (b) one or more times, each time with a different characterizing gene to generate one or more additional lines of transgenic animals,
thereby generating said collection of lines of transgenic animal, wherein each of said transgenic animals also comprises said second sequences operably linked to an expression control element activatable or repressible by said activator or repressor.

62. The method of claim 61 wherein said second sequences are contained within said transgene.

63. The method of claim 61 wherein said second sequences are not contained within said transgene.

64. The method of claim 63 wherein said second sequences are introduced into the genome of said transgenic animal by breeding.

65. A collection of vectors for making transgenic animals, said collection comprising two or more of said vectors wherein each of said vectors comprises a transgene, said transgene comprising (a) first sequences coding for a selectable or detectable marker protein and (b) regulatory sequences of a characterizing gene corresponding to an endogenous gene or ortholog of an endogenous gene operably linked to said first sequences such that when said transgene is present in the genome of a transgenic animal said first sequences are expressed in said transgenic animal with an expression pattern that is substantially the same as the expression pattern of said endogenous gene in a non-transgenic animal or anatomical region thereof, wherein the characterizing gene is different for each of said vectors.

66. The collection of vectors of claim 65 which comprises ten or more vectors.

67. The collection of vectors of claim 65 which comprises fifty or more vectors.

68. The collection of vectors of claim 65 wherein said transgene further comprises a coding sequence of said characterizing gene.

69. The collection of vectors of claim 68 wherein said first sequences are inserted or replaces sequences 5′ of said coding sequence of said characterizing gene.

70. The collection of vectors of claim 65 wherein said first sequences are operably linked to an IRES sequence that is not operably linked to a coding sequence of said characterizing gene.

71. The collection of vectors of claim 68 wherein said first sequences are fused in frame to the ATG start codon of said coding sequence of said characterizing gene.

72. The collection of vectors of claim 65 wherein said characterizing gene is not functionally expressed from said transgene.

73. The collection of vectors of claim 65 wherein said first sequences encode a detectable enzyme.

74. The collection of vectors of claim 73 wherein said detectable enzyme is β-lactamase.

75. The collection of vectors of claim 65 wherein said first sequences encode a fluorescent protein.

76. The collection of vectors of claim 75 wherein fluorescent protein is a GFP.

77. The collection of vectors of claim 65 wherein each said endogenous gene is specifically expressed in a subset of neurons.

78. The collection of vectors of claim 65 wherein each said endogenous gene is expressed in the same tissue.

79. The collection of vectors of claim 65 wherein each said endogenous gene is endogenously expressed in neuronal cells.

80. The collection of vectors of claim 65 wherein each of said endogenous genes endogenously expresses a protein product that is a part of an adrenergic or noradrenergic neurotransmitter pathway, a cholinergic neurotransmitter pathway, a dopaminergic neurotransmitter pathway, a GABAergic neurotransmitter pathway, a glutaminergic neurotransmitter pathway, a glycinergic neurotransmitter pathway, a histaminergic neurotransmitter pathway, a neuropeptidergic neurotransmitter pathway, a serotonergic neurotransmitter pathway, or the sonic hedgehog signaling pathway, is a nucleotide receptor, an ion channel, a marker of undifferentiated or not fully differentiated nerve cells, a calcium binding protein, or a neurotrophic factor receptor.

81. The collection of vectors of claim 65 wherein all of said endogenous genes are functionally related.

82. The collection of vectors of claim 65 wherein each of said endogenous genes is implicated in the same physiological or disease state.

83. The collection of vectors of claim 82 wherein the physiological or disease state is a neurological or psychiatric disease.

84. The collection of vectors of claim 83 wherein the neurological or psychiatric disease is schizophrenia, schizotypal personality disorder, psychosis, a schizoaffective disorder manic type disorder, a bipolar affective disorder, a bipolar affective (mood) disorder with hypomania and major depression (BP-II), a unipolar affective disorder, unipolar major depressive disorder, dysthymic disorder, a obsessive-compulsive disorder, a phobia, a panic disorder, a generalized anxiety disorder, a somatization disorder, hypochondriasis, or an attention deficit disorder.

85. The collection of vectors of claim 65 wherein each of said endogenous genes is a member of a group of genes that are implicated in the same physiological or behavioral response.

86. The collection of vectors of claim 85 wherein said physiological or behavioral response is pain, sleeping, feeding, fasting, sexual behavior or aggression.

87. The collection of vectors of claim 65 wherein each of said endogenous genes is expressed in neuronal cells involved in regulation of feeding behavior.

88. The collection of vectors of claim 65 wherein each of said endogenous genes is expressed in a different tissue.

89. The collection of vectors of claim 65 wherein each of said endogenous genes is implicated in a different physiological or disease state.

90. The collection of vectors of claim 65 wherein each of said endogenous genes is implicated in a different physiological or behavioral response.

91. The collection of vectors of claim 65 wherein said vectors are BACs.

92. A collection of vectors for making transgenic animals, said collection comprising two or more of said vectors wherein each of said vectors comprises a transgene, said transgene comprising (a) first sequences coding for an activator or repressor of expression of second sequences and (b) regulatory sequences of a characterizing gene corresponding to an endogenous gene or ortholog of an endogenous gene operably linked to said first sequences such that when said transgene is present in the genome of a transgenic animal said first sequences are expressed in said transgenic animal with an expression pattern that is substantially the same as the expression pattern of said endogenous gene in a non-transgenic animal or anatomical region thereof, wherein the characterizing gene is different for each of said vectors.

93. The collection of vectors of claim 92 wherein said second sequences are contained within said transgene.

94. The collection of vectors of claim 92 wherein said second sequences are not contained within said transgene.

95. A method of making a collection of vectors for making transgenic animals said collection comprising two or more of said vectors, said method comprising
(a) constructing a vector comprising a transgene, said transgene comprising (a) first sequences coding for a selectable or detectable marker protein and (b) regulatory sequences of a characterizing gene corresponding to an endogenous gene or ortholog of an endogenous gene operably linked to said first sequences such that when said transgene is present in the genome of a transgenic animal said first sequences are expressed in said transgenic animal with an expression pattern that is substantially the same as the expression pattern of said endogenous gene in a non-transgenic animal or anatomical region thereof, and
(b) repeating step (a) one or more times wherein each time step (a) is repeated a different characterizing gene is used;
thereby generating a collection of vectors for making transgenic animals.

96. The method of claim 95 in which said first sequences are introduced into said vector by homologous recombination.

97. The method of claim 96 which is carried out in E. coli cells.

98. The method of claim 95 wherein said vectors are BACs.

99. The method of claim 95 wherein said collection comprises ten or more vectors.

100. The method of claim 95 wherein said collection comprises fifty or more vectors.

101. The method of claim 95 wherein said transgene further comprises a coding sequence of said characterizing gene.

102. The method of claim 101 wherein said first sequences are inserted or replace sequences 5′ of said coding sequence of said characterizing gene.

103. The method of claim 95 wherein said first sequences are operably linked to an IRES sequence that is not operably linked to a coding sequence of said characterizing gene.

104. The method of claim 101 wherein said first sequences are fused in frame to the ATG start codon of said coding sequence of said characterizing gene.

105. The method of claim 95 wherein said characterizing gene is not functionally expressed from said transgene.

106. The method of claim 95 wherein said first sequences encode a detectable enzyme.

107. The method of claim 106 wherein said detectable enzyme is β-lactamase.

108. The method of claim 95 wherein said first sequences encode a fluorescent protein.

109. The method of claim 108 wherein fluorescent protein is a GFP.

110. The method of claim 95 wherein each said endogenous gene is expressed in the same tissue.

111. The method of claim 95 wherein each said endogenous gene is specifically expressed in a subset of neurons.

112. The method of claim 95 wherein each said endogenous gene is endogenously expressed in neuronal cells.

113. The method of claim 95 wherein each of said endogenous genes endogenously expresses a protein product that is a part of an adrenergic or noradrenergic neurotransmitter pathway, a cholinergic neurotransmitter pathway, a dopaminergic neurotransmitter pathway, a GABAergic neurotransmitter pathway, a glutaminergic neurotransmitter pathway, a glycinergic neurotransmitter pathway, a histaminergic neurotransmitter pathway, a neuropeptidergic neurotransmitter pathway, a serotonergic neurotransmitter pathway, or the sonic hedgehog signaling pathway, is a nucleotide receptor, an ion channel, a marker of undifferentiated or not fully differentiated nerve cells, a calcium binding protein, or a neurotrophic factor receptor.

114. The method of claim 95 wherein all of said endogenous genes are functionally related.

115. The method of claim 95 wherein each of said endogenous genes are implicated in the same physiological or disease state.

116. The method of claim 115 wherein the physiological or disease state is a neurological or psychiatric disease.

117. The method of claim 116 wherein the neurological or psychiatric disease is schizophrenia, schizotypal personality disorder, psychosis, a schizoaffective disorder manic type disorder, a bipolar affective disorder, a bipolar affective (mood) disorder with hypomania and major depression (BP-II), a unipolar affective disorder, unipolar major depressive disorder, dysthymic disorder, a obsessive-compulsive disorder, a phobia, a panic disorder, a generalized anxiety disorder, a somatization disorder, hypochondriasis, or an attention deficit disorder.

118. The method of claim 95 wherein each of said endogenous genes is implicated in the same physiological or behavioral response.

119. The method of claim 118 wherein said physiological or behavioral response is pain, sleeping, feeding, fasting, sexual behavior or aggression.

120. The method of claim 95 wherein each of said endogenous genes is expressed in neuronal cells involved in regulation of feeding behavior.

121. The method of claim 95 wherein each of said endogenous genes is expressed in a different tissue.

122. The method of claim 95 wherein each of said endogenous genes is implicated in a different physiological or disease state.

123. The method of claim 95 wherein each of said endogenous genes is implicated in the a different physiological or behavioral response.

124. A transgenic animal comprising a transgene, said transgene comprising (a) first sequences coding for a selectable or detectable marker protein; and (b) regulatory sequences of a characterizing gene corresponding to an endogenous gene or ortholog of an endogenous gene operably linked to said first sequences such that said first sequences are expressed in said transgenic animal with an expression pattern that is substantially the same as the expression pattern of said endogenous gene in a non-transgenic animal or anatomical region thereof, wherein said transgene is present in the genome at a site other than where the endogenous gene is located, said characterizing gene being ADRB1, ADRB2, ADRB3, ADRA1A, ADRA1B, ADRA1C, ADRA1D, ADRA2A, ADRA2B, ADRA2C, SLC6A2, Norepinephrine transporter, CHRM 1 (Muscarinic Ach M1) receptor, CHRM2 (Muscarinic Ach M2) receptor, CHRM3 (Muscarinic Ach M3) receptor, CHRM4 (Muscarinic Ach M4) receptor, CHRM5 (Muscarinic Ach M5) receptor, CHRNA1 (nicotinic alpha1) receptor, CHRNA2 (nicotinic alpha2) receptor, CHRNA3 (nicotinic alpha3) receptor, CHRNA4 (nicotinic alpha4) receptor, CHRNA5 (nicotinic alpha5) receptor, CHRNA7 (nicotinic alpha7) receptor, CHRN-B1 (nicotinic Beta 1) receptor, CHRNB2 (nicotinic Beta 2) receptor, CHRNB3 (nicotinic Beta 3) receptor, CHRNB4 (nicotinic Beta 4) receptor, CHRNG nicotinic gamma immature muscle receptor, CHRNE nicotinic epsilon receptor, CHRND nicotinic delta receptor, tyrosine hydroxylase, dopamine transporter, dopamine receptor 1, dopamine receptor 2, dopamine receptor 3, dopamine receptor 4, dopamine receptor 5, dbh, dopamine beta hydroxylase, GABA receptor A2, GABA receptor A3, GABA receptor A4, GABA receptor A5, GABA receptor A6, GABA receptor B1, GABA receptor B2, GABA receptor B3, GABA-A receptor (gamma 1 subunit), GABA-A receptor (gamma 2 subunit), GABA-A receptor (gamma 3 subunit), GABA-A receptor (delta subunit), GABA-A receptor (epsilon subunit), GABA-A receptor (pi subunit), GABA receptor theta, GABA receptor rho 1, GluR1, GlurR2, GluR3, GluR4, GluR5, GluR6, GluR7, GRIK4 (KA1), GRIK5 (KA2), NMDA receptor 1, NMDA receptor 2A, NMDA receptor 2B, NMDA receptor 2C, NMDA receptor 2D, mGluR1a, mGluR2, mGluR3, mGluR4, mGluR5, mGluR6, mGluR7, mGluR8, glut ionotropic delta, glutamate/aspartate transporter II, glutamate transporter GLT1, glutamate transporter SLC1A2, glial high affinity glutamate transporter, neuronal/epithelial high affinity glutamate transporter, glial high affinity glutamate transporter, high affinity aspartate/glutamate transporter, Glycine receptors alpha 1, Glycine receptors alpha 2, Glycine receptors alpha 3, Glycine receptors alpha 4, glycine receptor beta, histamine H1-receptor 1, Histamine H2-receptor 2, Histamine H3-receptor 3, orexin OX-A, Orexin receptor OX1R, Orexin receptor OX2R, Leptin receptor long form, melanin concentrating hormone, melanocortin 3 receptor, melanocortin 4 receptor, melanocortin 5 receptor, corticotropin releasing hormone, CRH/CRF receptor 1, CRH/CRF receptor 2, CRF binding protein, Urocortin, Pro-opiomelanocortin, cocaine and amphetamine regulated transcript, Neuropeptide Y, Neuropeptide Y1 receptor, Neuropeptide Y2 receptor, Npy4R Neuropeptide Y4 receptor, Npy5R Neuropeptide Y5 receptor, Npy6r Neuropeptide Y receptor, cholecystokinin, CCKAR cholecystokinin receptor, CCKBR cholecystokinin receptor, agouti related peptide, Galanin, Galanin like peptide, galanin receptorl, galanin receptor2, galanin receptor3, prepro-urotensin II, Urotensin receptor, somatostatin, somatostatin receptor sst1, somatostatin receptor sst2, somatostatin receptor sst3, somatostatin receptor sst4, somatostatin receptor sst5, G protein-coupled receptor 7, opioid-somatostatin-like receptor, G protein-coupled receptor 8 opioid-somatostatin-like receptor, pre Pro Enkephalin, Pre pro Dynorphin, μ opiate receptor, kappa opiate receptor, delta opiate receptor, ORL1 opioid receptor-like receptor, Vanilloid receptor subtype 1, protein 1 VRL 1, vanilloid receptor-like protein 1, vanilloid receptor-related osmotically activated channel, cannaboid receptors CB1, endothelin 1 ET-1 growth hormone releasing hormone, growth hormone releasing hormone receptor, nociceptin orphanin FQ/nocistatin, neuropeptide FF precursor, G-protein coupled receptor NPGPR, gastrin releasing peptide, preprogastrin-releasing peptide, gastrin releasing peptide receptor BB2, neuromedin B, neuromedin B receptor BB1, bombesin like receptor subtype-3, uterine bombesin receptor, GCG PROglucagon, glucagon receptor, GLP 1 receptor, GLP2 receptor, vasoactive intestinal peptide, secretin, pancreatic polypeptide receptor 1, pre-pro-Oxytocin, oxytocin receptor, Preprovasopressin, vasopressin receptor 1a, vasopressin receptor 1b, vasopressin receptor 2, Neurotensin tridecapeptide plus neuromedin N, Neurotensin receptor NT 1, Neurotensin receptor NT2, sortilin 1 neurotensin receptor 3, Bradykinin receptor 1, Bradykinin receptor B2, gonadotrophin releasing hormone, gonadotrophin releasing hormone, gonadotrophin releasing hormone receptor, calcitonin-related polypeptide, beta, calcitonin/calcitonin-related polypeptide alpha, calcitonin receptor, neurokinin A, neurokinin B, neurokinin a (subK) receptor, tachykinin receptor NK2 (Sub P and K), tachykinin receptor NK3 (Sub P and K) neuromedin K, PACAP, atrial naturietic peptide (ANP) precursor, atrial naturietic peptide (BNP) precursor, naturietic peptide receptor 1, naturietic peptide receptor 2, naturietic peptide receptor 3, VIP receptor 1, PACAP receptor, serotonin receptor 1A, serotonin receptor 2A, serotonin receptor 3, serotonin receptor 1B, serotonin receptor 1D, serotonin receptor 1E, serotonin receptor 2B, serotonin receptor 2C, serotonin receptor 4, serotonin receptor 5A, serotonin receptor 5B, serotonin receptor 6, serotonin receptor 7, serotonin transporter, tryptophan hydroxylase, purinergic receptor P2X ligand-gated ion channel, purinergic receptor P2X ligand-gated ion channel 3, purinergic receptor P2X ligand-gated ion channel 4, purinergic receptor P2X ligand-gated ion channel 5, purinergic receptor P2X-like 1 orphan receptor, purinergic receptor P2X ligand-gated ion channel 7, purinergic receptor P2Y G-protein coupled 1, purinergic receptor P2Y G-protein coupled 2, pyrimidinergic receptor P2Y G-protein coupled 4, pyrimidinergic receptor P2Y G-protein coupled 6, purinergic receptor P2Y G-protein coupled 11, voltage gated sodium channel type I alpha, sodium channel voltage-gated type I beta, sodium channel voltage-gated type II beta, sodium channel voltage-gated type V alpha, sodium channel voltage-gated type II alpha 1, sodium channel voltage-gated type II alpha 2, sodium channel voltage-gated type III alpha, sodium channel voltage-gated type IV alpha, sodium channel voltage-gated type VII or VI, sodium channel voltage-gated type VIII, sodium channel voltage-gated type IX alpha, sodium channel voltage-gated type X, sodium channel voltage-gated type XI alpha, sodium channel voltage-gated type XII alpha, sodium channel nonvoltage-gated 1 alpha, sodium channel voltage-gated type IV beta, sodium channel nonvoltage-gated 1 beta, sodium channel nonvoltage-gated 1 delta, sodium channel nonvoltage-gated 1 gamma, chloride channel 1 skeletal muscle, chloride channel 2, chloride channel 3, chloride channel 4, chloride channel 5, chloride channel 6, chloride channel 7, chloride intracellular channel 1, chloride intracellular channel 2, chloride intracellular channel 3, chloride intracellular channel 5, chloride channel Kb, chloride channel Ka, chloride channel, calcium activated family member 1, chloride channel calcium activated family member 2, chloride channel calcium activated family member 3, chloride channel calcium activated family member 4, potassium voltage-gated channel shaker-related subfamily member 1, potassium voltage-gated channel shaker-related subfamily member 2, potassium voltage-gated channel shaker-related subfamily member 3, potassium voltage-gated channel shaker-related subfamily member 4, potassium voltage-gated channel shaker-related subfamily member 4-like, potassium voltage-gated channel shaker-related subfamily member 5, potassium voltage-gated channel shaker-related subfamily member 6, potassium voltage-gated channel shaker-related subfamily member 7, potassium voltage-gated channel shaker-related subfamily member 10, potassium voltage-gated channel Shab-related subfamily member 1, potassium voltage-gated channel Shab-related subfamily member 2, potassium voltage-gated channel Shaw-related subfamily member 1, potassium voltage-gated channel Shaw-related subfamily member 2, potassium voltage-gated channel Shaw-related subfamily member 3, potassium voltage-gated channel Shaw-related subfamily member 4, potassium voltage-gated channel Shal-related family member 1, potassium voltage-gated channel Shal-related subfamily member 2, potassium voltage-gated channel Shal-related subfamily member 3, potassium voltage-gated channel Isk-related family member 1, potassium voltage-gated channel Isk-related family member 1-like, potassium voltage-gated channel Isk-related family member 2, potassium voltage-gated channel Isk-related family member 3, potassium voltage-gated channel Isk-related family member 4, potassium voltage-gated channel subfamily F member 1, potassium voltage-gated channel subfamily G member 1, potassium voltage-gated channel subfamily G member 2, potassium voltage-gated channel subfamily H (eag-related) member 1, potassium voltage-gated channel subfamily H (eag-related) member 2, potassium voltage-gated channel subfamily H (eag-related) member 3, potassium voltage-gated channel subfamily H (eag-related) member 4, potassium voltage-gated channel subfamily H (eag-related) member 5, potassium inwardly-rectifying channel subfamily J member 1, potassium inwardly-rectifying channel subfamily J member 2, potassium inwardly-rectifying channel subfamily J member 3, potassium inwardly-rectifying channel subfamily J member 4, potassium inwardly-rectifying channel subfamily J member 5, potassium inwardly-rectifying channel subfamily J member 6. potassium inwardly-rectifying channel subfamily J member 8, potassium inwardly-rectifying channel subfamily J member 9, potassium inwardly-rectifying channel subfamily J member 10, potassium inwardly-rectifying channel subfamily J member 11, potassium inwardly-rectifying channel subfamily J member 12, potassium inwardly-rectifying channel subfamily J member 13, potassium inwardly-rectifying channel subfamily J member 14, potassium inwardly-rectifying channel subfamily J member 15, potassium inwardly-rectifying channel subfamily J member 1, potassium channel, subfamily K member 1, potassium channel subfamily K member 2, potassium channel subfamily K member 3, potassium inwardly-rectifying channel subfamily K member 4, potassium channel subfamily K member 5, potassium channel subfamily K member 6, potassium channel subfamily K member 7, potassium channel subfamily K member 8, potassium channel subfamily K member 9, potassium channel subfamily K member 10, potassium intermediate/small conductance calcium-activated channel subfamily N member 1, potassium intermediate/small conductance calcium-activated channel subfamily member 2, potassium intermediate/small conductance calcium-activated channel subfamily N member 4, potassium voltage-gated channel KQT-like subfamily member 1, potassium voltage-gated channel KQT-like subfamily member 2, potassium voltage-gated channel KQT-like subfamily member 3, potassium voltage-gated channel KQT-like subfamily member 4, potassium voltage-gated channel KQT-like subfamily member 5, potassium voltage-gated channel delayed-rectifier, subfamily S member 1, potassium voltage-gated channel, delayed-rectifier, subfamily S member 2, potassium voltage-gated channel delayed-rectifier subfamily S member 3, potassium voltage-gated channel shaker-related subfamily beta member 1, potassium voltage-gated channel shaker-related subfamily beta member 2, potassium voltage-gated channel shaker-related subfamily beta member 3, potassium inwardly-rectifying channel subfamily J inhibitor 1, potassium large conductance calcium-activated channel subfamily M alpha member 1, potassium large conductance calcium-activated channel subfamily M alpha member 3, potassium large conductance calcium-activated channel subfamily M beta member 1, potassium large conductance calcium-activated channel subfamily M beta member 2, potassium large conductance calcium-activated channel subfamily M beta member 3-like, potassium large conductance calcium-activated channel, potassium large conductance calcium-activated channel sub M beta 4, hyperpolarization activated cyclic nucleotide-gated potassium channel 1, calcium channel voltage-dependent L type alpha 1S subunit, calcium channel voltage-dependent L type alpha 1C subunit, calcium channel voltage-dependent L type alpha 1D subunit, calcium channel voltage-dependent L type alpha 1F subunit, type calcium channel voltage-dependent P/Q tLype alpha 1A subunit, calcium channel voltage-dependent L type alpha 1B subunit, calcium channel voltage-dependent alpha 1E subunit, calcium channel voltage-dependent alpha 1G subunit, calcium channel, voltage-dependent alpha 1H subunit, calcium channel voltage-dependent alpha 1I subunit, NES (nestin), scip, sonic hedgehog, Smoothened Shh receptor, Patched Shh binding protein, calbindin d28 K, calretinin, parvalbumin, Trk B, GFR alpha 1, GFRalpha 2, GFRalpha 3, Neurotrophin receptor, Neurotrophin receptor, or Neurotrophic factor receptor.

125. The transgenic animal of claim 124 wherein said transgene further comprises a coding sequence of said characterizing gene.

126. The transgenic animal of claim 125 wherein said first sequences are inserted or replace sequences 5′ of said coding sequence of said characterizing gene.

127. The transgenic animal of claim 124 wherein said first sequences are operably linked to an IRES sequence that is not operably linked to a coding sequence of said characterizing gene.

128. The transgenic animal of claim 125 wherein said first sequences are fused in frame to the ATG start codon of said coding sequence of said characterizing gene.

129. The transgenic animal of claim 124 wherein said characterizing gene is not functionally expressed from said transgene.

130. The transgenic animal of claim 124 wherein said first sequences encode a detectable enzyme.

131. The transgenic animal of claim 130 wherein said detectable enzyme is β-lactamase.

132. The transgeniic animal of claim 124 wherein said first sequences encode a fluorescent protein.

133. The transgenic animal of claim 133 wherein fluorescent protein is a green fluorescent protein (GFP).

134. A transgenic animal comprising two or more transgenes, each said transgene comprising (a) first sequences coding for a selectable or detectable marker protein; and (b) regulatory sequences of a characterizing gene corresponding to an endogenous gene or ortholog of an endogenous gene operably linked to said first sequences such that said first sequences are expressed in said transgenic animal with an expression pattern that is substantially the same as the expression pattern of said endogenous gene in a non-transgenic animal or anatomical region thereof, wherein the characterizing gene is different for each said transgenes, and wherein each said transgene is present in the genome at a site other than where the endogneous gene is located.

135. The transgenic animal of claim 134 comprising 5 or more of said transgenes.

136. A method of isolating a collection of pure populations of cells wherein said collection comprises at least two different populations of cells, said method comprising isolating from two or more transgenic animals from the collection of transgenic animals of claim 1 or claim 28 the cells expressing said selectable or detectable marker from cells not expressing said selectable or detectable marker.

137. The method of claim 136 wherein said transgenic animals are transgenic mice.

138. The method of claim 136 wherein said collection comprises ten or more populations of cells.

139. The method of claim 136 wherein said collection comprises fifty or more populations of cells.

140. The method of claim 136 wherein said first sequences encode a detectable enzyme.

141. The method of claim 140 wherein said detectable enzyme is β-lactamase.

142. The method of claim 136 wherein said first sequences encode a fluorescent protein.

143. The method of claim 142 wherein fluorescent protein is a GFP.

144. The method of claim 142 wherein said isolating is by fluorescence activated cell sorting (FACS).

145. The method of claim 136 which further comprises culturing said isolated populations of cells.

146. A collection of pure populations of cells isolated from the transgenic animals of the collection of lines of transgenic animals of claim 1 or 28, wherein said cells express said detectable or selectable marker and each of said pure pure populations is isolated from a transgenic animal having a different characterizing gene.

147. A method of screening a candidate molecule for an effect on one or more cell types, said method comprising
(a) contacting said molecule to cells from each pure population of cells in the collection of claim 146; and
(b) detecting a change in said cells in response to said contacting.

148. The method of claim 147 wherein said change is measured by electrophysiology.

149. The method of claim 147 wherein said change is a change in gene expression.

150. The method of claim 149 wherein said change in gene expression is detected by hybridization of mRNA isolated from said cells to a microarray.

151. The method of claim 147 wherein said change is a change in cell morphology, cell proliferation, contact inhibition, or DNA replication.

152. The method of claim 147 wherein each pure population of cells in said collection was isolated from the transgenic animal which had been bred to a disease model of the same species or in which a disease state had been induced.

153. A method of screening a candidate molecule for an effect on one or more cell types, said method comprising
(a) administering said candidate molecule to a transgenic animal from each line of transgenic animals of the collection of transgenic animals of claim 1;
(b) isolating a pure population of cells from each of said transgenic animals that express said first sequences from the cells that do not express said first sequences; and
(c) detecting a change in said pure populations of cells from said transgenic animals adminstered said candidate molecule in comparison to corresponding pure populations of cells from transgenic animals from said lines of transgenic animals not administered said candidate molecule.

154. The method of claim 153 wherein said change is measured by electrophysiology.

155. The method of claim 153 wherein said change is a change in gene expression.

156. The method of claim 155 wherein said change in gene expression is detected by hybridization of mRNA isolated from said cells to a microarray.

157. The method of claim 153 wherein said change is a change in cell morphology, cell proliferation, contact inhibition, or DNA replication.

158. The method of claim 153 wherein each said transgenic animal had been bred to a disease model of the same species or in which a disease state had been induced.


SEQUENCE LISTING (1 sequence)

SEQ ID NO: 1 - DNA, 551 bp, EMCV
taacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctaagcgtattcaacaaggggctgaaggatgcccagaaggtactccattgtatgggatctgatctggggcctcggtgcacatgctttacatgtgtttagtcgaggttaaaaaaacgtctaggccccccgaaccacggggacgtggttttcctttgaaaaacaccatgata